(19) United States
(12) Patent Application Publication
(10) Pub. No.: US 2024/0392016 A1
(43) Pub. Date: Nov. 28, 2024

(54) USE OF CD123 NK CELL ENGAGER FOR TREATING AML AND RELATED DISORDERS

(71) Applicant: SANOFI, Paris (FR)

(72) Inventors: Peter Adamson, Cambridge, MA (US); Celine Amara, Paris (FR); Alexandra Basset, Paris (FR); Brigitte Demers, Paris (FR); Dobrin Draganov, Cambridge, MA (US); Paula Fraenkel, Cambridge, MA (US); Kyle Jensen, Cambridge, MA (US); Choi Siak-Leng, Paris (FR); Delphine Valente, Paris (FR); Samira Ziti-Ljajic, Paris (FR)

(21) Appl. No.: 18/644,287

(22) Filed: Apr. 24, 2024

Related U.S. Application Data
(60) Provisional application No. 63/559,491, filed on Feb. 29, 2024.
(60) Provisional application No. 63/597,622, filed on Nov. 9, 2023.
(60) Provisional application No. 63/538,210, filed on Sep. 13, 2023.
(60) Provisional application No. 63/529,919, filed on Jul. 31, 2023.
(60) Provisional application No. 63/528,509, filed on Jul. 24, 2023.
(60) Provisional application No. 63/526,661, filed on Jul. 13, 2023.
(60) Provisional application No. 63/468,634, filed on May 24, 2023.
(60) Provisional application No. 63/465,218, filed on May 9, 2023.
(60) Provisional application No. 63/464,404, filed on May 5, 2023.
(60) Provisional application No. 63/461,480, filed on Apr. 24, 2023.

(51) Int. Cl. C07K 16/28; A61P 35/02

(52) U.S. Cl. CPC C07K 16/2866; A61P 35/02; C07K 16/2803; C07K 2317/31

(57) ABSTRACT

Provided herein are methods comprising multifunctional binding proteins comprising a first and a second antigen binding domain and all or part of an immunoglobulin Fc region or variant thereof, wherein the first antigen binding domain binds specifically to CD123 and the second antigen binding domain binds specifically to human NKp46, and wherein all or part of the immunoglobulin Fc region or variant thereof bind to a human Fc-γ receptor, for the treatment or prevention of leukemias and myelodysplastic disorders.

### CROSS-REFERENCE TO RELATED APPLICATIONS

This application claims priority to U.S. Provisional Patent Application Ser. No. 63/461,480, filed Apr. 24, 2023; 63/464,404, file May 5, 2023; 63/465,218, filed May 9, 2023; 63/468,634, filed May 24, 2023; 63/526,661, filed Jul. 13, 2023; 63/528,509, filed Jul. 24, 2023; 63/529,919, filed Jul. 31, 2023; 63/538,210, filed Sep. 13, 2023; 63/597,622, filed Nov. 9, 2023; and 63/559,491, filed Feb. 29, 2024; the contents of which are hereby incorporated by reference in their entireties.

### SEQUENCE LISTING

The instant application contains a Sequence Listing which has been submitted electronically in XML format and is hereby incorporated by reference in its entirety. Said XML file, created on Aug. 13, 2024, is named 752288_SA9-366_ST26.xml and is 112,390 bytes in size.

### BACKGROUND

Acute myeloid leukemia (AML), B cell acute lymphoblastic leukemia (B-ALL), and myelodysplastic syndromes (MDS) are heterogeneous clonal neoplastic disease, which are thought to arise from subpopulations of leukemic stem cells, which tend to be resistant to convention chemotherapy, and which may be further responsible for disease relapse. Patients diagnosed with relapsed or refractory HR-MDS face a bleak median overall survival (OS) following unsuccessful treatment with hypomethylating agents (HMA).

Blastic plasmacytoid dendritic cell neoplasm (BPDCN) is a rare, aggressive hematological malignancy arising from plasmacytoid dendritic cell precursors. In the past, several different nomenclatures have been used to describe BPDCN until 2008 when the World Health Organization (WHO) classification described BPDCN as an entity under the family of acute myeloid leukemia (AML) and related neoplasms. It was later given its own separate category under myeloid neoplasms in the WHO re-classification, reflecting its unique pathobiology. BPDCN generally presents with cutaneous lesions with or without bone marrow involvement, lymphadenopathy, splenomegaly, cytopenias, and sometimes with extramedullary involvement.

Natural killer (NK) cells are a subpopulation of lymphocytes that are involved in non-conventional immunity. NK cells provide an efficient immunosurveillance mechanism by which undesired cells such as tumor- or virally-infected cells can be eliminated. Characteristics and biological properties of NK cells include the expression of surface antigens including CD16, CD56 and/or CD57, the absence of the α/β or γ/δ TCR complex on the cell surface, the ability to bind to and kill cells in a MHC-unrestrictive manner and in particular cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate the immune response.

Still, there is an urgent need for methods of treatment for patients who are ineligible for or have exhausted standard therapeutic options.

### SUMMARY

In one aspect, provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46, wherein the binding protein is administered to the subject at a dose of at least 3 g/kg.

In another aspect, provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively;
wherein the binding protein is administered to the subject at a dose of at least 3 μg/kg.

In some embodiments, the dose is between about 3 μg/kg and about 6000 μg/kg. In some embodiments, the dose is between about 3 μg/kg and about 30 μg/kg. In some embodiments, the dose is between about 10 μg/kg and 100 μg/kg. In some embodiments, the dose is between about 30 μg/kg and 100 μg/kg. In some embodiments, the dose is between about 13 μg/kg and about 130 μg/kg. In some embodiments, the dose is between about 15 μg/kg and about 150 μg/kg. In some embodiments, the dose is between about 20 μg/kg and about 200 μg/kg. In some embodiments, the dose is between about 30 μg/kg and about 300 μg/kg. In some embodiments, the dose is between about 40 μg/kg and about 400 μg/kg. In some embodiments, the dose is between about 45 μg/kg and about 450 μg/kg. In some embodiments, the dose is between about 60 μg/kg and about 600 μg/kg. In some embodiments, the dose is between about 100 μg/kg and about 1000 μg/kg. In some embodiments, the dose is between about 300 μg/kg and about 1000 μg/kg. In some embodiments, the dose is between 400 μg/kg and about 1300 μg/kg. In some embodiments, the dose is between about 450 μg/kg and about 1500 μg/kg. In some embodiments, the dose is between about 600 μg/kg and about 1000 μg/kg. In some embodiments, the dose is between about 600 μg/kg and about 1500 μg/kg. In some embodiments, the dose is between about 600 μg/kg and about 2000 μg/kg. In some embodiments, the dose is between about 1000 μg/kg and about 3000 μg/kg. In some embodiments, the dose is between about 1300 μg/kg and about 3000 μg/kg. In some embodiments, the dose is between about 1500 μg/kg and about 3000 μg/kg. In some embodiments, the dose is between about 2000 μg/kg and about 3000 μg/kg.

In some embodiments, the dose is about 3 μg/kg. In some embodiments, the dose is about 10 μg/kg. In some embodiments, the dose is about 13 μg/kg. In some embodiments, the dose is about 15 μg/kg. In some embodiments, the dose is about 20 μg/kg. In some embodiments, the dose is about 30 μg/kg. In some embodiments, the dose is about 40 μg/kg. In some embodiments, the dose is about 45 μg/kg. In some embodiments, the dose is about 50 μg/kg. In some embodiments, the dose is about 60 μg/kg. In some embodiments, the dose is about 100 μg/kg. In some embodiments, the dose is about 130 μg/kg. In some embodiments, the dose is about 150 μg/kg. In some embodiments, the dose is about 200 μg/kg. In some embodiments, the dose is about 300 μg/kg. In some embodiments, the dose is about 400 μg/kg. In some embodiments, the dose is about 450 μg/kg. In some embodiments, the dose is about 500 μg/kg. In some embodiments, the dose is about 600 μg/kg. In some embodiments, the dose is about 750 μg/kg. In some embodiments, the dose is about 1000 μg/kg. In some embodiments, the dose is about 1300 μg/kg. In some embodiments, the dose is about 1500 μg/kg. In some embodiments, the dose is about 2000 μg/kg. In some embodiments, the dose is about 3000 μg/kg. In some embodiments, the dose is about 4000 μg/kg. In some embodiments, the dose is about 4500 μg/kg. In some embodiments, the dose is about 6000 μg/kg.

In some embodiments, the binding protein is administered to the subject intravenously, subcutaneously, intraperitoneally, or intramuscularly. In some embodiments, the binding protein is administered to the subject intravenously.

In some embodiments, the binding protein is administered to a subject for at least one cycle. In some embodiments, the at least one cycle is about 28 days. In some embodiments, the binding protein is administered to a subject for at least two cycles. In some embodiments, the binding protein is administered to a subject for three or more cycles.

In some embodiments, the first antigen binding domain with binding specificity to CD123 comprises:

a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and

a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the second antigen binding domain with binding specificity to NKp46 comprises:
a. a second heavy chain variable domain (VH2) comprising a CDR-H1, H2, and H3 corresponding to the amino acid sequences of:
   i. SEQ ID NO: 13, 14, and 15, respectively;
   ii. SEQ ID NO: 16, 17, and 18, respectively;
   iii. SEQ ID NO: 19, 20, and 21, respectively;
   iv. SEQ ID NO: 22, 23, and 24, respectively; or
   v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the VH2 comprises an amino acid sequence of SEQ ID NO: 45, and wherein the VL1 comprises an amino acid sequence of SEQ ID NO: 53; the VH2 comprises an amino acid sequence of SEQ ID NO: 46, and wherein the VL2 comprises an amino acid sequence of SEQ ID NO: 54; the VH2 comprises an amino acid sequence of SEQ ID NO: 47, and wherein the VL2 comprises an amino acid sequence of SEQ ID NO: 55; the VH2 comprises an amino acid sequence of SEQ ID NO: 48, and wherein the VL2 comprises an amino acid sequence of SEQ ID NO: 56; the VH2 comprises an amino acid sequence of SEQ ID NO: 49, and wherein the VL2 comprises an amino acid sequence of SEQ ID NO: 57; the VH2 comprises an amino acid sequence of SEQ ID NO: 50, and wherein the VL2 comprises an amino acid sequence of SEQ ID NO: 58; the VH2 comprises an amino acid sequence of SEQ ID NO: 51, and wherein the VL2 comprises an amino acid sequence of SEQ ID NO: 59; or the VH2 comprises an amino acid sequence of SEQ ID NO: 52, and wherein the VL2 comprises an amino acid sequence of SEQ ID NO: 60.

In some embodiments, the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1H} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO: 75;
Hinge₃ corresponds to the amino acid sequence of SEQ ID NO: 77;
L₁ corresponds to the amino acid sequence of SEQ ID NO: 76.

In some embodiments, residue N297 of the Fc region or variant thereof according to EU numbering comprises a N-linked glycosylation.

In some embodiments, all or part of the Fc region or variant thereof binds to a human CD16A (FcγRIII) polypeptide.

In some embodiments, at least two polypeptide chains are linked by at least one disulfide bridge. In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}. In some embodiments, V_{1A} is V_{L1} and V_{1B} is V_{H1}. In some embodiments, V_{2A} is V_{H2} and V_{2B} is V_{L2}.

In some embodiments, V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;

V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;

V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;

V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;

V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;

V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;

V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;

V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;

V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or

V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, the V_{H1} and V_{L1} corresponds to the amino acid sequences of SEQ ID NO: 41 and 43 respectively or corresponds to the amino acid sequences of SEQ ID NO: 42 and 44 respectively; and/or V_{H2} and V_{L2} corresponds to the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively.

In some embodiments, V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53; V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54; V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55; V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60; V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57; V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58; V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, polypeptide (I) consists of an amino acid sequence of SEQ ID NO: 64; polypeptide (II) consists of an amino acid sequence of SEQ ID NO: 65; and polypeptide (III) consists of an amino acid sequence of SEQ ID NO: 66.

In some embodiments, the leukemia is acute myeloid leukemia (AML). In some embodiments, the AML is relapsed or refractory. In some embodiments, the leukemia is B cell acute lymphoblastic leukemia (B-ALL). In some embodiments, the hematological disease or disorder is a myelodysplastic syndrome. In some embodiments, the myelodysplastic syndrome is a high-risk myelodysplastic syndrome (HR-MDS).

In an aspect, provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46,
wherein the first antigen binding domain comprises a heavy chain variable domain (VH1) and a light chain variable domain (VL1), wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9; and
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15; and
   the VL2 comprises a CDR-L1, L2, and L3 corresponding to the amino acid sequences of SEQ ID NO: 27, 28, and 29;
and wherein all or part of the immunoglobulin Fc region or variant thereof binds to a human Fc-γ receptor,
wherein the binding protein is administered to the subject at a dose of at least 3 μg/kg.

In another aspect, provided herein is a method of treating or preventing a leukemia or a myelodysplastic disorder in a subject in need thereof, the method comprising administering to the subject a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66,
   wherein the binding protein is administered to the subject at a dose of at least 3 μg/kg.

In another aspect, provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46.

In one aspect, provided herein is a method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In one aspect, provided herein is a method of treating or preventing a leukemia or a hematological neoplasm in a pediatric subject in need thereof, the method comprising administering to the subject a binding protein comprising a first antigen binding domain with binding specificity to CD123 and a second antigen binding domain with binding specificity to NKp46.

In another aspect, provided herein is a method of treating or preventing blastic plasmacytoid dendritic cell neoplasm (BPDCN) in a subject in need thereof, the method comprising administering to the subject a binding protein comprising a first antigen binding domain with binding specificity to CD123 and a second antigen binding domain with binding specificity to NKp46.

In yet another aspect, provided herein is a method of treating or preventing blastic plasmacytoid dendritic cell neoplasm (BPDCN) in a subject in need thereof, the method comprising administering to the subject a binding protein comprising a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the subject is a pediatric subject.

In some embodiments, the binding protein is administered to the subject intravenously, subcutaneously, intraperitoneally, or intramuscularly. In some embodiments, the binding protein is administered to the subject intravenously.

In some embodiments, the binding protein is administered at dose between about 500 μg/kg and about 12000 μg/kg. In some embodiments, the binding protein is administered at a dose between about 600 μg/kg and about 1000 μg/kg. In some embodiments, the binding protein is administered at a dose between about 600 μg/kg and about 1500 μg/kg. In some embodiments, the dose is about 500 μg/kg. In some embodiments, the dose is about 600 μg/kg. In some embodiments, the dose is about 1000 μg/kg. In some embodiments, the dose is about 1500 μg/kg. In some embodiments, the dose is about 3000 μg/kg. In some embodiments, the dose is about 6000 μg/kg. In some embodiments, the dose is about 12000 μg/kg.

In some embodiments, the first antigen binding domain with binding specificity to CD123 comprises a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the second antigen binding domain with binding specificity to NKp46 comprises: a second heavy chain variable domain (VH2) comprising a CDR-H1, H2, and H3 corresponding to the amino acid sequences of: SEQ ID NO: 13, 14, and 15, respectively; SEQ ID NO: 16, 17, and 18, respectively; SEQ ID NO: 19, 20, and 21, respectively; SEQ ID NO: 22, 23, and 24, respectively; or SEQ ID NO: 16, 25, and 26, respectively; and
a second light chain variable domain (VL2) comprising a CDR-L1, L2, and L3 corresponding to the amino acid sequences of: SEQ ID NO: 27, 28, and 29, respectively; SEQ ID NO: 30, 31, and 32, respectively; SEQ ID NO: 33, 34, and 35, respectively; SEQ ID NO: 36, 37, and 38, respectively; or SEQ ID NO: 39, 31, and 40, respectively.

In some embodiments, the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the the VH2 comprises an amino acid sequence of SEQ ID NO: 45, and wherein the VL1 comprises an amino acid sequence of SEQ ID NO: 53; the VH2 comprises an amino acid sequence of SEQ ID NO: 46, and wherein the VL2 comprises an amino acid sequence of SEQ ID NO: 54; the VH2 comprises an amino acid sequence of SEQ ID NO: 47, and wherein the VL2 comprises an amino acid sequence of SEQ ID NO: 55; the VH2 comprises an amino acid sequence of SEQ ID NO: 48, and wherein the VL2 comprises an amino acid sequence of SEQ ID NO: 56; the VH2 comprises an amino acid sequence of SEQ ID NO: 49, and wherein the VL2 comprises an amino acid sequence of SEQ ID NO: 57; the VH2 comprises an amino acid sequence of SEQ ID NO: 50, and wherein the VL2 comprises an amino acid sequence of SEQ ID NO: 58; the VH2 comprises an amino acid sequence of SEQ ID NO: 51, and wherein the VL2 comprises an amino acid sequence of SEQ ID NO: 59; or the VH2 comprises an amino acid sequence of SEQ ID NO: 52, and wherein the VL2 comprises an amino acid sequence of SEQ ID NO: 60.

In some embodiments, the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2—C_{H}3)_{A} corresponds to the amino acid sequence of SEQ ID NO: 69;
(C_{H}2—C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO: 75;
Hinge₃ corresponds to the amino acid sequence of SEQ ID NO: 77;
L₁ corresponds to the amino acid sequence of SEQ ID NO: 76.

In some embodiments, residue N297 of the Fc region or variant thereof according to EU numbering comprises a N-linked glycosylation. In some embodiments, the all or part of the Fc region or variant thereof binds to a human CD16A (FcγRIII) polypeptide. In some embodiments, the binding protein comprises at least two polypeptide chains linked by at least one disulfide bridge.

In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, V_{1A} is VL1 and V_{1B} is V_{H1}. In some embodiments, V_{2A} is V_{H2} and V_{2B} is V_{L2}.

In some embodiments, V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, V_{H1} and V_{L1} corresponds to the amino acid sequences of SEQ ID NO: 41 and 43 respectively or corresponds to the amino acid sequences of SEQ ID NO: 42 and 44 respectively; and/or V_{H2} and V_{L2} corresponds to the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively.

In some embodiments, V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53; V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54; V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55; V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60; V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57; V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58; V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, polypeptide (I) consists of an amino acid sequence of SEQ ID NO: 64; polypeptide (II) consists of an amino acid sequence of SEQ ID NO: 65; and polypeptide (III) consists of an amino acid sequence of SEQ ID NO: 66.

In an aspect, provided herein is a method of treating or preventing a leukemia in a pediatric subject in need thereof, the method comprising administering to the subject a binding protein comprising a first antigen binding domain with binding specificity to CD123 and a second antigen binding domain with binding specificity to NKp46.

In some embodiments, the leukemia is acute myeloid leukemia (AML). In some embodiments, the AML is relapsed or refractory. In some embodiments, the leukemia is B cell acute lymphoblastic leukemia (B-ALL).

In some embodiments, the binding protein is administered to the subject intravenously, subcutaneously, intraperitoneally, or intramuscularly. In some embodiments, the binding protein is administered to the subject intravenously.

In some embodiments, the first antigen binding domain with binding specificity to CD123 comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the second antigen binding domain with binding specificity to NKp46 comprises: a second heavy chain variable domain (VH2) comprising a CDR-H1, H2, and H3 corresponding to the amino acid sequences of: SEQ ID NO: 13, 14, and 15, respectively; SEQ ID NO: 16, 17, and 18, respectively; SEQ ID NO: 19, 20, and 21, respectively; SEQ ID NO: 22, 23, and 24, respectively; or SEQ ID NO: 16, 25, and 26, respectively; and a second light chain variable domain (VL2) comprising a CDR-L1, L2, and L3 corresponding to the amino acid sequences of: SEQ ID NO: 27, 28, and 29, respectively; SEQ ID NO: 30, 31, and 32, respectively; SEQ ID NO: 33, 34, and 35, respectively; SEQ ID NO: 36, 37, and 38, respectively; or SEQ ID NO: 39, 31, and 40, respectively.

In some embodiments, the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54; the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55; the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56; the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57; the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58; the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the VH2 comprises an amino acid sequence of SEQ ID NO: 45, and wherein the VL1 comprises an amino acid sequence of SEQ ID NO: 53; the VH2 comprises an amino acid sequence of SEQ ID NO: 46, and wherein the VL2 comprises an amino acid sequence of SEQ ID NO: 54; the VH2 comprises an amino acid sequence of SEQ ID NO: 47, and wherein the VL2 comprises an amino acid sequence of SEQ ID NO: 55; the VH2 comprises an amino acid sequence of SEQ ID NO: 48, and wherein the VL2 comprises an amino acid sequence of SEQ ID NO: 56; the VH2 comprises an amino acid sequence of SEQ ID NO: 49, and wherein the VL2 comprises an amino acid sequence of SEQ ID NO: 57; the VH2 comprises an amino acid sequence of SEQ ID NO: 50, and wherein the VL2 comprises an amino acid sequence of SEQ ID NO: 58; the VH2 comprises an amino acid sequence of SEQ ID NO: 51, and wherein the VL2 comprises an amino acid sequence of SEQ ID NO: 59; or the VH2 comprises an amino acid sequence of SEQ ID NO: 52, and wherein the VL2 comprises an amino acid sequence of SEQ ID NO: 60.

In some embodiments, the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO: 75;
Hinge₃ corresponds to the amino acid sequence of SEQ ID NO: 77;
   L₁ corresponds to the amino acid sequence of SEQ ID NO: 76.

In some embodiments, residue N297 of the Fc region or variant thereof according to EU numbering comprises a N-linked glycosylation. In some embodiments, the all or part of the Fc region or variant thereof binds to a human CD16A (FcγRIII) polypeptide. In some embodiments, the binding protein comprises at least two polypeptide chains linked by at least one disulfide bridge.

In some embodiments, the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, V_{1A} is V_{L1} and V_{1B} is V_{H1}. In some embodiments, V_{2A} is V_{H2} and V_{2B} is V_{L2}.

In some embodiments, V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35; V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, V_{H1} and V_{L1} corresponds to the amino acid sequences of SEQ ID NO: 41 and 43 respectively or corresponds to the amino acid sequences of SEQ ID NO: 42 and 44 respectively; and/or V_{H2} and V_{L2} corresponds to the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively.

In some embodiments, V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53; V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54; V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55; V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60; V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57; V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58; V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59; V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60; V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53; V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54; V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55; V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56; V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57; V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58; V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, polypeptide (I) consists of an amino acid sequence of SEQ ID NO: 64; polypeptide (II) consists of an amino acid sequence of SEQ ID NO: 65; and polypeptide (III) consists of an amino acid sequence of SEQ ID NO: 66.

In an aspect, provided herein is a method of treating or preventing blastic plasmacytoid dendritic cell neoplasm (BPDCN) in a subject in need thereof, the method comprising administering to the subject a binding protein comprising a first antigen binding domain with binding specificity to CD123 and a second antigen binding domain with binding specificity to NKp46,
wherein the first antigen binding domain comprises a heavy chain variable domain (VH1) and a light chain variable domain (VL1), wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9; and
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15; and
   the VL2 comprises a CDR-L1, L2, and L3 corresponding to the amino acid sequences of SEQ ID NO: 27, 28, and 29;
   and wherein all or part of the immunoglobulin Fc region or variant thereof binds to a human Fc-γ receptor.

In another aspect, provided herein is method of treating or preventing blastic plasmacytoid dendritic cell neoplasm (BPDCN) in a pediatric subject in need thereof, the method comprising administering to the subject a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66.

In yet another aspect, provided herein is a method of treating or preventing blastic plasmacytoid dendritic cell neoplasm (BPDCN) in a pediatric subject in need thereof, the method comprising administering to the subject a binding protein comprising a first antigen binding domain with binding specificity to CD123 and a second antigen binding domain with binding specificity to NKp46,
wherein the first antigen binding domain comprises a heavy chain variable domain (VH1) and a light chain variable domain (VL1), wherein:
   the VH1 comprises a complementary determining region (CDR)-H1, H2 and H3 corresponding to the amino acid sequences of SEQ ID NO: 1, 2, and 3; and
   the VL1 comprises a CDR-L1, L2 and L3 corresponding to the amino acid sequences of SEQ ID NO: 7, 8, and 9; and
wherein the second antigen binding domain comprises a heavy chain variable domain (VH2) and a light chain variable domain (VL2), wherein:
   the VH2 comprises a CDR-H1, H2, and H3 corresponding to the amino acid sequences of SEQ ID NO: 13, 14, and 15; and
   the VL2 comprises a CDR-L1, L2, and L3 corresponding to the amino acid sequences of SEQ ID NO: 27, 28, and 29;
and wherein all or part of the immunoglobulin Fc region or variant thereof binds to a human Fc-γ receptor.

In another aspect, provided herein is a method of treating or preventing blastic plasmacytoid dendritic cell neoplasm (BPDCN) in a subject in need thereof, the method comprising administering to the subject a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that consists of an amino acid sequence of SEQ ID NO: 64;
a polypeptide (II) that consist of an amino acid sequence of SEQ ID NO: 65; and
a polypeptide (III) that consists of an amino acid sequence of SEQ ID NO: 66.

In an aspect, provided herein is a method of treating or preventing acute myeloid leukemia (AML) in a pediatric subject in need thereof, the method comprising administering to the subject a binding protein comprising a first antigen binding domain with binding specificity to CD123 and a second antigen binding domain with binding specificity to NKp46.

In some embodiments, the pediatric subject with AML is a between the ages of about 1 year old and about 11 years old.

In an aspect, provided herein is a method of treating or preventing acute myeloid leukemia (AML) in a pediatric subject in need thereof, the method comprising administering to the subject a binding protein comprising a first antigen binding domain with binding specificity to CD123 and a second antigen binding domain with binding specificity to NKp46, wherein the pediatric subject is between the ages of about 1 year old and about 11 years old.

In yet another aspect, provided herein is a method of treating or preventing acute myeloid leukemia (AML) in a pediatric subject in need thereof, the method comprising administering to the subject a binding protein comprising a first antigen binding domain with binding specificity to CD123 and a second antigen binding domain with binding specificity to NKp46, wherein the first antigen binding domain comprises a heavy chain variable domain (VH1) and a light chain variable domain (VL1), wherein: the VH1 comprises a complementary determining region (CDR)-H1, H2 and H3 corresponding to the amino acid sequences of SEQ ID NO: 1, 2, and 3; and the VL1 comprises a CDR-L1, L2 and L3 corresponding to the amino acid sequences of SEQ ID NO: 7, 8, and 9; and wherein the second antigen binding domain comprises a heavy chain variable domain (VH2) and a light chain variable domain (VL2), wherein: the VH2 comprises a CDR-H1, H2, and H3 corresponding to the amino acid sequences of SEQ ID NO: 13, 14, and 15; and the VL2 comprises a CDR-L1, L2, and L3 corresponding to the amino acid sequences of SEQ ID NO: 27, 28, and 29; and wherein all or part of the immunoglobulin Fc region or variant thereof binds to a human Fc-γ receptor, and wherein the pediatric subject in need thereof is between the ages of about 1 year old and about 11 years old.

In another aspect, provided herein is a method of treating or preventing acute myeloid leukemia (AML) in a pediatric subject in need thereof, the method comprising administering to the subject a binding protein comprising a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises: a polypeptide (I) that consists of an amino acid sequence of SEQ ID NO: 64; a polypeptide (II) that consist of an amino acid sequence of SEQ ID NO: 65; and a polypeptide (III) that consists of an amino acid sequence of SEQ ID NO: 66, and wherein the pediatric subject in need thereof is between the ages of about 1 year old and about 11 years old.

In some embodiments, the pediatric subject is between the ages of about 1 year old and about 17 years old. In some embodiments, the pediatric subject is between the ages of about 1 year old and about 11 years old. In some embodiments, the pediatric subject is about 1 year old. In some embodiments, the pediatric subject is about 2 years old. In some embodiments, the pediatric subject is about 3 years old. In some embodiments, the pediatric subject is about 4 years old. In some embodiments, the pediatric subject is about 5 years old. In some embodiments, the pediatric subject is about 4 years old. In some embodiments, the pediatric subject is about 6 years old. In some embodiments, the pediatric subject is about 7 years old. In some embodiments, the pediatric subject is about 8 years old. In some embodiments, the pediatric subject is about 9 years old. In some embodiments, the pediatric subject is about 10 years old. In some embodiments, the pediatric subject is about 11 years old. In some embodiments, the pediatric subject is about 12 years old. In some embodiments, the pediatric subject is about 13 years old. In some embodiments, the pediatric subject is about 14 years old. In some embodiments, the pediatric subject is about 15 years old. In some embodiments, the pediatric subject is about 16 years old. In some embodiments, the pediatric subject is about 17 years old.

### BRIEF DESCRIPTION OF THE DRAWINGS

If not specified otherwise, the binding proteins of the present disclosure are oriented with the amino terminal direction (“N-terminal end” or “N-term”) on the left-hand side and the carboxyl-terminal direction (“C-terminal end” or “C-term”) on the right-hand side, in accordance with standard usage and convention.

FIG. 1 depicts a tri-dimensional schematic representation of the F25 format which is a variant of the bispecific F5 format, including one human NKp46 binding site and one human CD123 binding site. As depicted in FIG. 1, the C-term of the polypeptide is on the left-hand side and the N-term is on the right-hand side.

FIG. 2A-2D illustrates two-dimensional schematic representations of F25, F5, F26 and F6 formats respectively including the relevant domains for each polypeptide chain. In FIG. 2A to 2D, the C-term of the polypeptide is on the left-hand side and the N-term is on the right-hand side. The human NKp46 binding domain is formed by the VH/VL pair on the left side. The human CD123 binding domain is formed by the VH/VL pair on the right side.

FIG. 2A shows a two-dimensional schematic representation of the F25 format. This representation represents the claimed “NKp46-CD123_F25” binding protein.

FIG. 2B shows a two-dimensional schematic representation of the F5 format. When compared to F25, the F5 differs in that the CL and CH pair of the NKp46 binding domain are swapped, with the third polypeptide chain comprising a CH1 domain and a VL domain.

FIG. 2C shows a two-dimensional schematic representation of the F26 format. This F26 differs from F25 of FIG. 2A in that it includes a Fc-silent N297S mutation on each CH2 domain.

FIG. 2D shows a two-dimensional schematic representation of the F6 format. This F6 differs from F5 of FIG. 2B in that it includes a Fc-silent N297S mutation on each CH2 domain.

FIG. 2E shows a two-dimensional detailed representation of a variant of the F25 format. This F25 format representation corresponds to the one in FIG. 2A.

FIG. 3 shows a proposed mechanism of action of a NK Cell Engager (NKCE) for killing, following joint binding of a tumor cell (i.e. an AML cell line; i.e. MOLM-13) expressing CD123 and a NK cell expressing NKp46 and a Fcγ receptor (CD16a). Reproduced and adapted from Gauthier, L. et al. (“Multifunctional natural killer cell engagers targeting NKp46 trigger protective tumor immunity”. Cell 177, 1701-1713 (2019)).

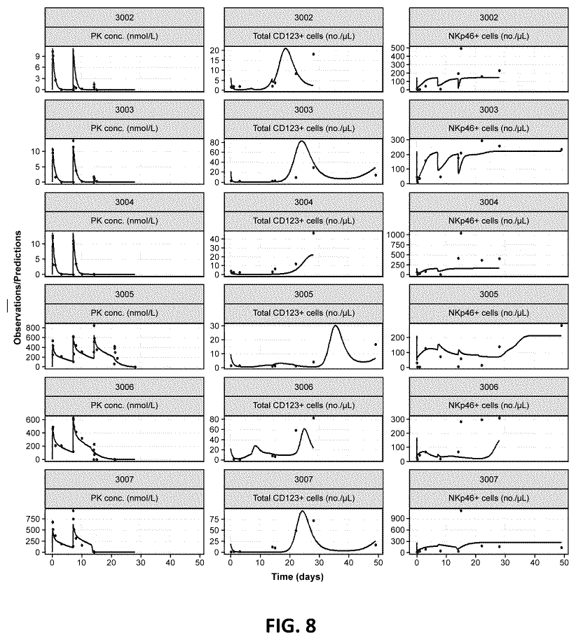

FIG. 4A-4C is a set of schematic diagrams illustrating synapse-driven PK/PD in vitro model in MOLM-13 cells (FIG. 4A), synapse-driven PK/PD in vivo NHP model (FIG. 4B), and a translational AML synapse-driven PK/PD model (FIG. 4C).

FIG. 5 depicts cytotoxicity (%) profiles of CD123 NKCE concentrations in the in vitro system stratified by incubation time and E:T ratio in a MOLM-13 in vitro mode. Solid lines represent typical model simulation; solid circles are individual observations of in vitro cytotoxicity

FIG. 6A-6C demonstrate the visual predictive checks of in vivo NHP PK/PD model for PK (FIG. 6A), total CD123+ cells (FIG. 6B), and NKp46+NK cells (FIG. 6C) for IV dosing of 3000 μg/kg SD, 100 and 3000 μg/kg QW, and SC dosing of 3.6 μg/kg SD, in addition PK data from IV dosing of 100 μg/kg and 3000 μg/kg b.i.w., and SC dosing of 3000 μg/kg b.i.w were also included.

FIG. 7A-7B are predictions of CD123 NKCE following an intra-patient dose-escalation scheme with a b.i.w. regimen, where 10 μg/kg and 30 μg/kg were the first and second doses in week 1, followed by 100 μg/kg (10/30/100), and a fixed dose with 100 μg/kg (fixed 100) b.i.w. regimen. FIG. 7A is a PK prediction of a time-course plot in semi-log scale. Dashed horizontal line represents the minimum concentration showing mild cytokine release in a whole-blood cytokine release assay (1.7 nmol/L). FIG. 7B is a % CFB in CD123+ cell count prediction. dashed horizontal lines represent change from baseline at 0 and −95% (target level defined for efficacy).

FIG. 8 illustrates individual model fits of the NHP PK/PD model from representative individuals. The dots represent observations and the lines represent individual predictions.

FIG. 9 is a set of plots demonstrating the external validation of the in vivo NHP PK/PD model simulation by comparing with observations of total CD123+ and NKp46+NK cell counts following b.i.w. dosing, while PK data from this dosing regimen were fitted to estimate PK parameters. Solid lines represent typical model simulation, and the shaded area represents the 90% prediction interval (N=100). Solid circles represent the mean of the observations with standard deviation as error bars. Observed PK data (red solid circles) were used to estimate PK parameters, whereas observed total CD123+ and NKp46+NK cell counts (blue solid circles) were compared with the model-simulated profiles. Note: CD123+ cell count data >200 cells/μL were excluded.

FIG. 10A-10B are global (GSA) and local sensitivity analyses (LSA). FIG. 10A uses extended Fourier amplitude sensitivity test (eFAST) to evaluate sensitivity of parameters (k_{g}, alpha [α], kᵢₙTP, gamma [γ], kₘₐₓ, Beta [β], kᵢₙₜ, EC₅₀, TCₘₐₓ, RTdE, EF₀, TA₀, kₒᵤₜ, Slp_{NK}, RTdT, KD1, KD2, k_{off1}, and k_{off2}) in the translational AML PK/PD model with the predicted AUC of CD123+ cells at selected FIH starting dose (10/30/100 μg/kg). FIG. 10B (LSA) evaluates the sensitive parameters identified in GSA with the percent change from baseline of CD123+ cell count profiles at the FIH starting dose.

FIG. 11 illustrates Kaplan-Meier curves and survival differences between mice treated with the CD123 NKCE and an anti-CD123 antibody-drug conjugate.

FIG. 12 depicts Kaplan-Meier curves for muNKp46-huCD123, X-huCD123 isotype control, and a muNKp46-X isotype control-treated NK-depleted SCID mice.

FIG. 13A-13C illustrate results of responders at various points after treatment. The top chart depicts patients given CD123 NKCE at DL3 and DL4. Two responders remain in remission (CR) after 8.8 and 12.2 months of treatment. The lower chart depicts patients at other dose levels.

FIG. 14A, 14B, 14C, 14D depict leukemic blast count in whole blood (FIG. 14A and FIG. 14D) and bone marrow (FIG. 14B and FIG. 14C) before and after treatment with CD123 NKCE.

FIGS. 15A and 15B illustrate the Cₘₐₓ (FIG. 15A) and C_{trough} (FIG. 15B) of CD123 NKCE at DL1-DL6 in patients (measured as ng/mL) throughout Cycle 1.

FIGS. 16A and 16B illustrate mean plasma concentration during Cycle 1 at dose levels DL1-DL6. Linearity was achieved at weekly doses of 3000 μg/kg (DL5 and DL6).

FIG. 17 illustrates plasma cytokine levels during Cycle 1 in dose levels DL1-DL6. Key pro-inflammatory cytokine levels peaked 1 hour to 3 hours after the first dose, and returned to baseline by 24 hours.

FIG. 18A-18C depict an adult escalation treatment scheme (FIG. 18A) and expansion treatment scheme (FIG. 18B). FIG. 18C depicts the pediatric escalation/expansion/optimization treatment schema.

FIG. 19 illustrates the study design for each cohort (Cohorts A, B, C, and D) in both the dose escalation and expansion/optimization parts in both the adult study (top) and the pediatric study (bottom).

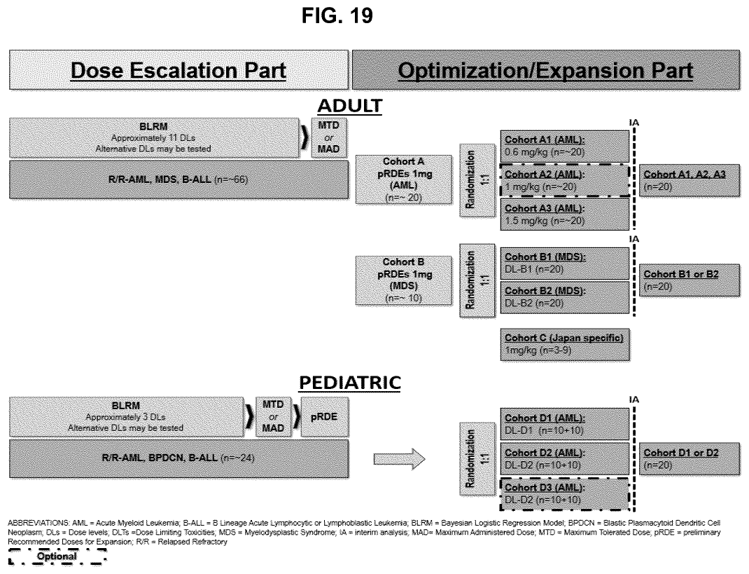

FIG. 20 depicts cytotoxicity (% lysis) of MOLM-12, THP-1, OCI-AML2, and NB-4 cells incubated with a range of CD123 NKCE or NKp46×CD16-X isotype control concentrations.

FIG. 21 is a set of simulated CD123 NKCE plasma concentrations versus time profiles, overlaid with observed data over the first induction cycle.

FIG. 22 is a set of simulated synapse concentrations versus time profiles over the first induction cycle.

FIG. 23 illustrates individual predicted normalized synapse exposure over the first induction cycle by dose level (n=58 patients).

FIG. 24 is the predicted response rate (CR/CRi or BR) by dose level.

FIG. 25 illustrates the predicted synapse exposure by dose level.

### DETAILED DESCRIPTION

This disclosure provides methods of treating hematological diseases and disorders (e.g., relapsed or refractory acute myeloid leukemia (AML), B cell acute lymphoblastic lymphoma (B-ALL), high risk myelodysplasia, or blastic plasmacytoid dendritic cell neoplasm (BPDCN)) comprising multifunctional binding proteins that bind one surface biomarker on immune NK cells, i.e., NKp46 and one antigen of interest on tumoral target cells, i.e., CD123, and is capable of redirecting NK cells to lyse a target cell that expresses the CD123 surface biomarker. The multifunctional binding proteins of the present disclosure further comprises all or part of a Fc region or variant thereof which binds a Fc-γ receptor (FcγR), in particular an activating Fc-γ receptor (FcγR), for example FcγRIIIa also called CD16a.

### I. Definitions

That the disclosure may be more readily understood, select terms are defined below.

The articles “a” and “an” are used herein to refer to one or to more than one (i.e., to at least one) of the grammatical object of the article.

“About” or “approximately” as used herein when referring to a measurable value such as an amount, a temporal duration, and the like, is meant to encompass variations of ±20% or ±10%, ±5%, ±1%, or ±0.1% of a given value or range, as such variations are appropriate to perform the disclosed methods.

As used herein, the term “Cluster of Differentiation 123” or “CD123” marker is also known as “Interleukin 3 receptor alpha (IL3RA)” or “IL3R”, “IL3RX”, “IL3RY”, “IL3RAY”, “hIL-3Ra” and denotes an interleukin 3 specific subunit of a heterodimeric cytokine receptor. The functional interleukin 3 receptor is a heterodimer that comprises a specific alpha chain (IL-3A; CD123) and the IL-3 receptor beta chain (βθ; CD131) that is shared with the receptors for granulocyte macrophage colony stimulating factor (GM-CSF) and interleukin 5 (IL-5). CD123 is a type I integral transmembrane protein with a deduced Molecular Weight of about 43 kDa containing an extracellular domain involved in IL-3 binding, a transmembrane domain and a short cytoplasmic tail of about 50 amino acids. The extracellular domain is composed of two regions: a N-terminal region of about 100 amino acids, the sequence of which exhibits similarity to equivalent regions of the GM-CSF and IL-5 receptor alpha-chains; and a region proximal to the transmembrane domain that contains four conserved cysteine residues and a motif, common to other members of this cytokine receptor family. The IL-3 binding domain comprises about 200 amino acid residue cytokine receptor motifs (CRMs) made up of two Ig-like folding domains. The extracellular domain of CD123 is highly glycosylated, with N-glycosylation necessary for both ligand binding and receptor signaling. The protein family gathers three members: IL3RA (CD123A), CSF2RA and IL5RA. The overall structure is well conserved between the three members, but sequence homologies are very low. One 300 amino-acid long isoform of CD123 has been discovered so far, but only on the RNA level which is accessible on the Getentry database under the accession number ACM241 16.1. A reference sequence of full-length human CD123 protein, including signal peptide, is available from the NCBI database under the accession number NP_002174.1 and under the Uniprot accession number P26951.

The extracellular domain of human CD123 (ECD) consists of the amino acid sequence of SEQ ID NO: 82. CD123 (the interleukin-3 receptor alpha chain IL-3Ra) is a tumor antigen overexpressed in a variety of hematological neoplasms. The majority of AML blasts express surface CD123 and this expression does not vary by subtype of AML. Higher expression of CD123 on AML at diagnosis has been reported to be associated with poorer prognosis.

CD123 expression has been reported in other hematological malignancies including myelodysplasia, systemic mastocytosis, blastic plasmacytoid dendritic cell neoplasm (BPDCN), ALL and hairy cell leukemia.

As used herein, “Natural killer” or “NK cells” refers to a sub-population of lymphocytes that is involved in non-conventional immunity. NK cells can be identified by virtue of certain characteristics and biological properties, such as the expression of specific surface antigens including CD16, CD56 and/or CD57, NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell KS and Colonna M). Human Press. pp. 219-238 (2000).

As used herein, the term “NKp46” marker, or “Natural cytotoxicity triggering receptor 1”, also known as “CD335” or “NKP46” or “NK-p46” or “LY94” refers to a protein or polypeptide encoded by the Ncr1 gene. A reference sequence of full-length human NKp46 protein is available from the NCBI database under the accession number NP_004820. The human NKp46 extracellular domain (ECD) corresponds to the amino acid sequence of SEQ ID NO: 80. The human NKp46 mRNA sequence is described in NCBI accession number NM_004829.

As used herein, the term “Fc-γ receptor” or “FcγR” or “Fc-gamma receptor” may refer to both activating and inhibitory FcγRs. Fc-gamma receptors (FcγR) are cellular receptors for the Fc region of an Immunoglobulin G (IgG). Upon binding of complexed IgG, FcγRs can modulate cellular immune effector functions, thereby linking the adaptive and innate immune systems, including ADCC-mediated immune responses. In humans, six classic FcγRs are currently reported: one high-affinity receptor (FcγRI) and five low-to-medium-affinity FcγRs (FcγRIIA, -B and -C, FcγRIIIA and -B). All FcγRs bind the same region on IgG Fc, yet with differing high (FcgRI) and low (FcgRII and FcgRIII) affinities. On a functional level, most of the FcγRs are activating receptors that can induce the cellular responses mentioned above, including ADCC-mediated immune response. Whereas FcγRI, FcγRIIa, FcγRIIc, and FcγRIIIa are activating receptors characterized by an intracellular immunoreceptor tyrosine-based activation motif (ITAM), FcγRIIb has an inhibition motif (ITIM) and is therefore inhibitory. Unless specified otherwise, the term FcγRs encompasses activating receptors, including FcγRI (CD64), FcγRIIA (CD32a), FcγRIIIa (CD16a) and FcγRIIIb (CD16b), and preferably FcγRIIIa (CD16a).

As used herein, the terms “FcγRIIIa (CD16a)” or “FcγRIIIa” or “CD16a” or “CD16” or “Cluster of Differentiation 16” may refer to a 50-65 kDa cell surface molecule expressed on mast cells, macrophages, and natural killer cells as a transmembrane receptor. FcγRIIIa is an activating receptor containing immunoreceptor tyrosine activating motifs (ITAMs) in the associated FcR γ-chain, ITAMs being necessary for receptor expression, surface assembly and signaling. CD16a is a low affinity receptor for IgG and is an important receptor mediating ADCC (antibody dependent cell mediated cytotoxicity) by NK cells. The high affinity receptor CD16a is preferentially found on NK cells and monocytes and induces antibody-dependent cellular cytotoxicity (ADCC) upon IgG binding.

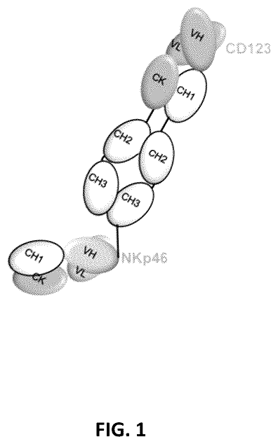

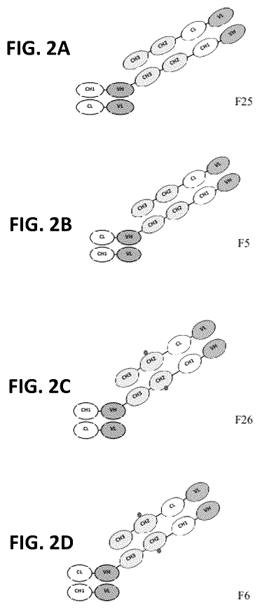

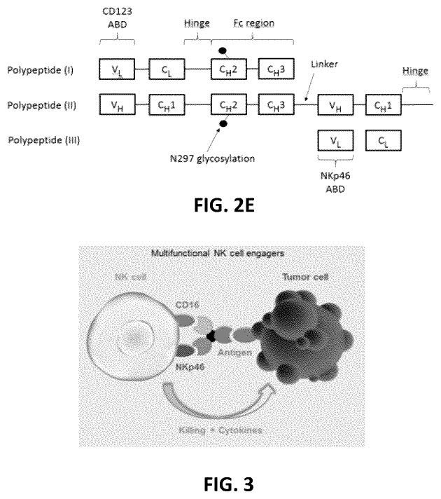

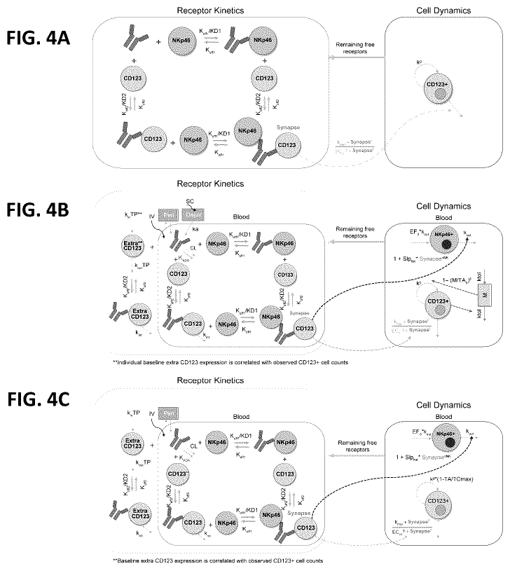

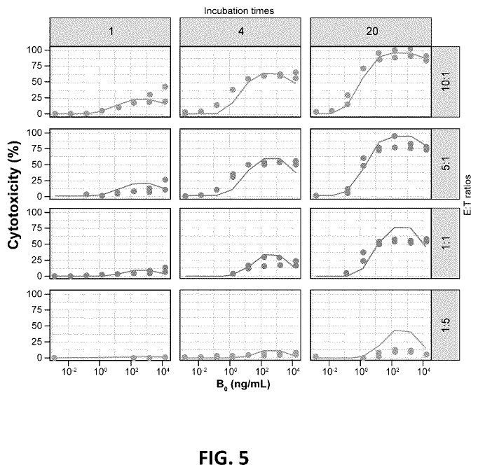

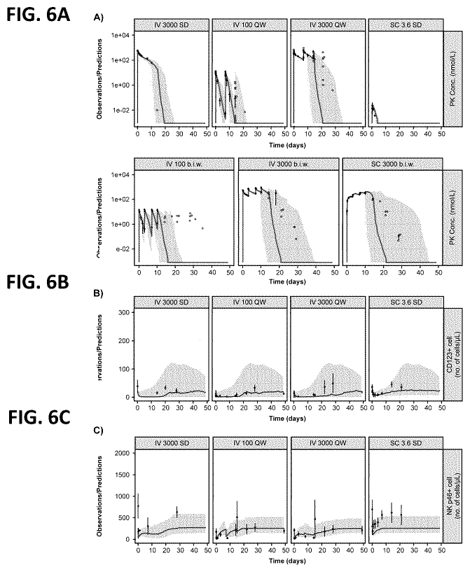

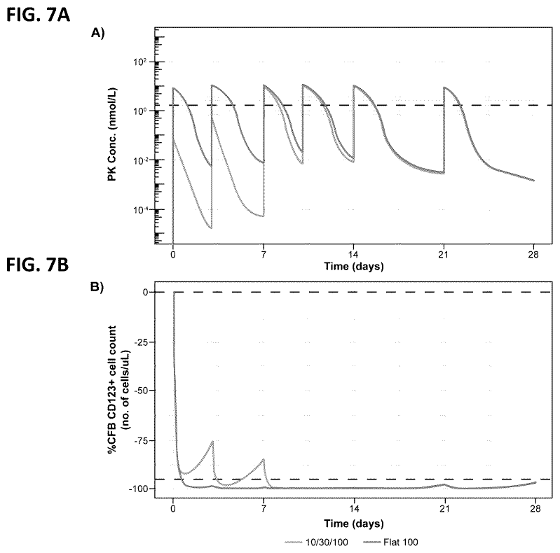

As used herein, the terms “Format 5” or “F5”, “Format 25” or “F25”, “Format F6” or “F6” and “Format 26” or “F26” refer to specific binding protein configurations of bispecific or multispecific antibodies specifically designed to engineer multiple antigen binding domains into a single antibody molecule. The multifunctional binding proteins of the present disclosure which comprise a NKp46-binding domain and a CD123-binding domain, are made based on the F25 format, as exemplified in FIG. 1 and FIG. 2. F25 and format F26 respectively differ from format F5 and F6 in that one CH1/CL pair between the second and third polypeptide chain are swapped to form a CL/CH1 pair. The F5 and F6 format have been previously described in the international patent application WO2017114694, incorporated herein by reference.

As used herein, the term “bispecific binding protein” refers to a binding protein that specifically binds to two different antigen targets (e.g., human NKp46 and human CD123) through at least two distinct antigen-binding domains (ABDs). A bispecific binding protein may be bivalent (two ABDs) or multivalent (more than two ABDs).

As used herein, the terms “specifically binds to” or “binds specifically to” refers to the ability of an antigen-binding domain (ABD) to bind to an antigen (e.g. human NKp46 and/or human CD123) containing an epitope with an Kd of at least about 1×10⁻⁶ M, 1×10⁻⁷ M, 1×10⁻⁸ M, 1×10⁻⁹ M, 1×10⁻¹⁰ M, 1×10⁻¹¹ M, 1×10⁻¹² M, or more, and/or to bind to an epitope with an affinity that is at least twofold greater than its affinity for a nonspecific antigen.

As used herein, the term “specifically binds to human NKp46 polypeptide” may refer to a specific binding toward a polypeptide comprising an amino acid sequence of SEQ ID NO: 80.

As used herein, the term “specifically to a human CD123 polypeptide” may refer to a specific binding toward a polypeptide comprising an amino acid sequence of SEQ ID NO: 82.

As used herein, the term “binds to a human Fc-γ receptor polypeptide” may refer to a binding toward a polypeptide comprising an amino acid sequence of SEQ ID NO: 83 or SEQ ID NO: 84.

Competitive binding assays and other methods for determining specific binding are further described below and are well known in the art. Expressions such as “specifically binds to”, or “with specificity for” are used interchangeably. Those terms are not construed to refer exclusively to those antibodies, polypeptides and/or multichain polypeptides which actually bind to the recited target/binding partner, but also to those which, although provided in a non-bound form, retain the specificity to the recited target. Binding specificity can be quantitatively determined by an affinity constant KA (or KA) and a dissociation constant KD (or K_{D}).

As used herein, the term “affinity”, concentration (EC₅₀) or the equilibrium dissociation constant (K_{D}) means the strength of the binding of an antibody or polypeptide to an epitope. The affinity of an antibody is given by a specific type of equilibrium constant, which is the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant KA is defined by 1/KD. Preferred methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of mAbs is the use of surface plasmon resonance (SPR) screening (such as by analysis with a BIAcore™ SPR analytical device). In a non-limitative manner, a KD of less than 50 nM as determined by SPR, and under physiological conditions (e.g. at a pH ranging from 6 to 8 under normal buffer conditions), may generally be considered as indicative of specificity of binding for antigen-antigen binding domain (ABD) interactions.

As an illustration, and according to some particular and exemplified embodiments, binding proteins reported herein comprise:
an antigen binding domain which binds specifically to human CD123 with a K_{D} of less than 10 nM, in particular with a K_{D} of less than 0.5 nM, as determined by SPR, under physiological conditions;
an antigen binding domain which binds specifically to human NKp46 with a K_{D} of less than 50 nM, in particular with a K_{D} of less than 20 nM, as determined by SPR, under physiological conditions.

As used herein, the term “and/or” is a grammatical conjunction that is to be interpreted as encompassing that one or more of the cases it connects may occur. For example, the wording “such native sequence proteins can be made using standard recombinant and/or synthetic methods” indicates that native sequence proteins can be made using standard recombinant and synthetic methods or native sequence proteins can be made using standard recombinant methods or native sequence proteins can be made using synthetic methods.

As used herein, “treating” refers to a therapeutic use (i.e., on a subject having a given disease) and means reversing, alleviating, inhibiting the progress of one or more symptoms of such disorder or condition. Therefore, treatment does not only refer to a treatment that leads to a complete cure of the disease, but also to treatments that slow down the progression of the disease and/or prolong the survival of the subject.

As used herein, “preventing” means a prophylactic use (i.e., on a subject susceptible of developing a given disease and encompasses the treatment of blastic plasmacytoid dendritic cell neoplasm (BPDCN) relapsed AML patient.

As used herein, the terms “therapeutically effective amount” of the multifunctional binding protein or pharmaceutical composition thereof is meant a sufficient amount of the antibody-like multifunctional binding protein to treat said cancer disease, at a reasonable benefit/risk ratio applicable to any medical treatment. It will be understood, however, that the total daily usage of the polypeptides and compositions of the present disclosure will be decided by the attending physician within the scope of sound medical judgment. The specific therapeutically effective dose level for any particular patient will depend upon a variety of factors including the disorder being treated and the severity of the disorder; activity of the specific polypeptide employed; the specific composition employed, the age, body weight, general health, sex and diet of the patient; the time of administration, route of administration, and rate of excretion of the specific polypeptide employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved.

As used herein, the term “subject” or “individual” or “patient” are used interchangeably and may encompass a human or a non-human mammal, rodent or non-rodent. The term includes, but is not limited to, mammals, e.g., humans including man, woman and child, other primates (monkey), pigs, rodents such as mice and rats, rabbits, guinea pigs, hamsters, cows, horses, cats, dogs, sheep and goats.

As used herein, the term “pediatric” as used herein refers to a child patient aged from one month to 18 years. The indicated age is to be understood as the age of the child patient at diagnosis of the disease or disorder. The children may be more specifically subgrouped into infants (1 to 12 months of age), younger children aged 1 to 9 years, and older children and adolescents (10 to 18 years of age). In some embodiments, the pediatric patient may be diagnosed with blastic plasmacytoid dendritic cell neoplasm (BPDCN). In some embodiments, the pediatric patient may be diagnosed with pediatric acute myeloid leukemia (AML). In some embodiments, the pediatric patient may be diagnosed with B cell acute lymphoblastic leukemia (B-ALL). In some embodiments, the pediatric patient may be diagnosed with high risk-myelodysplasia (HR-MDS). In some embodiments, the pediatric subject is between the ages of about 1 year old and about 17 years old. In some embodiments, the pediatric subject is between the ages of about 1 year old and about 11 years old. In some embodiments, the pediatric subject is about 1 year old. In some embodiments, the pediatric subject is about 2 years old. In some embodiments, the pediatric subject is about 3 years old. In some embodiments, the pediatric subject is about 4 years old. In some embodiments, the pediatric subject is about 5 years old. In some embodiments, the pediatric subject is about 4 years old. In some embodiments, the pediatric subject is about 6 years old. In some embodiments, the pediatric subject is about 7 years old. In some embodiments, the pediatric subject is about 8 years old. In some embodiments, the pediatric subject is about 9 years old. In some embodiments, the pediatric subject is about 10 years old. In some embodiments, the pediatric subject is about 11 years old. In some embodiments, the pediatric subject is about 12 years old. In some embodiments, the pediatric subject is about 13 years old. In some embodiments, the pediatric subject is about 14 years old. In some embodiments, the pediatric subject is about 15 years old. In some embodiments, the pediatric subject is about 16 years old. In some embodiments, the pediatric subject is about 17 years old.

As used herein, the singular form “a”, “an” and “the” include plural references unless the context clearly dictates otherwise. For example, the term “a pharmaceutically acceptable carrier” encompasses a plurality of pharmaceutically acceptable carriers, including mixtures thereof.

As used herein, “a plurality of” may thus include «two» or «two or more».

As used herein, “antibody” or “immunoglobulin” may refer to a natural or conventional antibody in which two heavy chains are linked to each other by disulfide bonds and each heavy chain is linked to a light chain by a disulfide bond. There are two types of light chain, lambda (λ) and kappa (κ). There are five main heavy chain classes (or isotypes) which determine the functional activity of an antibody molecule: IgM, IgD, IgG, IgA and IgE. Each chain contains distinct sequence domains. The light chain includes two domains or regions, a variable domain (VL) and a constant domain (CL). The heavy chain generally includes four domains, a variable domain (VH) and three constant domains (CH1, CH2 and CH3, collectively referred to as CH). In particular, classes IgG, IgA, and IgD have three heavy chain constant region domains, which are designated CH1 CH2, and CH3; and the IgM and IgE classes have four heavy chain constant region domains, CH1, CH2, CH3, and CH4. The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the antigen-binding fragment (Fab) of an immunoglobulin and consists of the variable portions of one light chain and one heavy chain.

As used herein, when referring to “IgG” or “Immunoglobulin G” in general, IgG1, IgG2, IgG3 and IgG4 are included, unless defined otherwise. In particular, IgG is IgG1.

As used herein, the term “antibody-like” or “immunoglobulin-like” polypeptide may also refer to non-conventional or synthetic antigen-binding polypeptides or binding protein, including single domain antibodies and fragments thereof, in particular variable heavy chain of single domain antibodies, and chimeric, humanized, bispecific or multimeric antibodies.

As used herein, the term “multifunctional binding protein” encompass a multi-chain protein, including but not limited to antibody-like polypeptide or protein formats, which comprises at least one first variable region (e.g. a first immunoglobulin heavy chain variable domain (VH) and/or an immunoglobulin light chain variable domain (VL)) binding specifically to a human CD123 polypeptide, and at least one second variable region (e.g. a second immunoglobulin heavy chain variable domain (VH) and/or immunoglobulin light chain variable domain (VL)) binding specifically to a human NKp46 polypeptide. Although not limited specifically to a particular type of construct, one general embodiment is particularly considered throughout the specification: the polypeptide constructs reported in WO2015197593 and WO2017114694, each of which is incorporated herein by reference. In particular, the multifunctional binding protein such as those reported in WO2015197593 and WO2017114694, may encompass any construct comprising one or more polypeptide chains.

As used herein, the term “humanized”, as in “humanized antibody” refers to a polypeptide (i.e., an antibody or an antibody-like polypeptide) which is wholly or partially of non-human origin and which has been modified to replace certain amino acids, in particular in the framework regions of the heavy and light chains, in order to avoid or minimize an immune response in humans. The constant domains of a humanized antibody are most of the time human CH and CL domains. Numerous methods for humanization of an antibody sequence are known in the art; see e.g., the review by Almagro & Fransson (2008) Front Biosci. 13: 1619-1633. One commonly used method is CDR grafting, or antibody reshaping, which involves grafting of the CDR sequences of a donor antibody, generally a mouse antibody, into the framework scaffold of a human antibody of different specificity.

For chimeric antibodies, humanization typically involves modification of the framework regions of the variable region sequences. Amino acid residues that are part of a CDR will typically not be altered in connection with humanization, although in certain cases it may be desirable to alter individual CDR amino acid residues, for example to remove a glycosylation site, a deamidation site or an undesired cysteine residue. N-linked glycosylation occurs by attachment of an oligosaccharide chain to an asparagine residue in the tripeptide sequence Asn-X-Ser or Asn-X-Thr, where X may be any amino acid except Pro. Removal of an N-glycosylation site may be achieved by mutating either the Asn or the Ser/Thr residue to a different residue, in particular by way of conservative substitution. Deamidation of asparagine and glutamine residues can occur depending on factors such as pH and surface exposure. Asparagine residues are particularly susceptible to deamidation, primarily when present in the sequence Asn-Gly, and to a lesser extent in other dipeptide sequences such as Asn-Ala. When such a deamidation site, in particular Asn-Gly, is present in a CDR sequence, it may therefore be desirable to remove the site, typically by conservative substitution to remove one of the implicated residues. Substitution in a CDR sequence to remove one of the implicated residues is also intended to be encompassed by the claimed multifunctional binding protein.

As used herein, the term “conservative amino acid substitution” refers to substitutions in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine), and aromatic side chains (e.g., tyrosine, phenylalanine, tryptophan). When an amino acid belongs to two different classes (i.e., tyrosine & phenylalanine), both can be accepted. As a reference, the following classification will be followed throughout the specification, unless stated otherwise.

| Conservative Substitution | Type of Amino acid |
|---|---|
| Ala, Val, Leu, Ile, | Amino acids with non-polar side |
| Met, Pro, Phe, Trp | chains |
| Ser, Thr, Tyr, Asn, | Amino acids with uncharged polar |
| Gln, Cys, Gly | side chains |
| Asp, Glu | Amino acids with acidic side chains |
| Lys, Arg, His | Amino acids with basic side chains |
| Tyr, Phe, Trp, | Aromatic amino acids |

As used herein, the term “domain” may be any region of a protein, generally defined on the basis of sequence homologies or identities, which is related to a specific structural or functional entity. Accordingly, the term “region”, as used in the context of the present disclosure, is broader in that it may comprise additional regions beyond the corresponding domain.

As used herein, the terms “linker region”, “linker peptide” or “linker polypeptide” or “amino acid linker” or “linker” refer to any amino acid sequence suitable for covalently linking two polypeptide domains, such as two antigen-binding domains together and/or a Fc region to one or more variable regions, such as one or more antigen-binding domains. Although the term is not limited to a particular size or polypeptide length, such amino acid linkers are generally less than 50 amino acids in length, preferably less than 30 amino acids in length, for instance 20 or less than 20 amino acids in length, for instance 15 or less than 15 amino acids in length. Such amino acid linkers may optionally comprise all or part of an immunoglobulin polypeptide chain, such as all or part of a hinge region of an immunoglobulin. Alternatively, the amino acid linker may comprise a polypeptide sequence that is not derived from a hinge region of an immunoglobulin, or even that is not derived from an immunoglobulin heavy or light polypeptide chain.

As used herein, an immunoglobulin hinge region, or a fragment thereof, may thus be considered as a particular type of linker, which is derived from an immunoglobulin polypeptide chain.

As used herein, the term “hinge region” or “hinge” refers to a generally flexible region and born by the corresponding heavy chain polypeptides, and which separates the Fc and Fab portions of certain isotypes of immunoglobulins, more particularly of the IgG, IgA or IgD isotypes. Such hinge regions are known in the Art to depend upon the isotype of immunoglobulin which is considered. For native IgG, IgA and IgD isotypes, the hinge region thus separates the CH1 domain and the CH2 domain and is generally cleaved upon papain digestion. On the other hand, the region corresponding to the hinge in IgM and IgE heavy chains is generally formed by an additional constant domain with lower flexibility. Additionally, the hinge region may comprise one or more cysteines involved in interchain disulfide bonds. The hinge region may also comprise one or more binding sites to a Fcγ receptor, in addition to FcγR binding sites born by the CH2 domain, when applicable. Additionally, the hinge region may comprise one or more post-translational modification, such as one or more glycosylated residues depending on the isotype which is considered. Thus, it will be readily understood that the reference to the term “hinge” throughout the specification is not limited to a particular set of hinge sequences or to a specific location on the structure. Unless instructed otherwise, the hinge regions which are still particularly considered comprise all or part of a hinge from an immunoglobulin belonging to one isotype selected from: the IgG isotype, the IgA isotype and the IgD isotype; in particular the IgG isotype.

As used herein, the terms “CH domain”, or “CH domain”, or “constant domain”, can be used interchangeably and refer to any one or more heavy chain immunoglobulin constant domain(s). Such CH domains are natively folded as immunoglobulin-like domains, although they may be partly disordered in an isolated form (e.g., CH1 domains when not associated with the constant domain of a light chain (CL)). Unless instructed otherwise, the term may thus refer to a CH1 domain, a CH2 domain, a CH3 domain; or any combinations thereof.

As used herein, the terms “CH1 domain”, or “CH1 domain”, or “constant domain 1”, can be used interchangeably and refer to the corresponding heavy chain immunoglobulin constant domain 1.

As used herein, the term “CH2 domain”, or “CH2 domain”, or “constant domain 2” can be used interchangeably and refer to the corresponding heavy chain immunoglobulin constant domain 2.

As used herein, the term “CH3 domain”, or “CH3 domain”, or “constant domain 3” can be used interchangeably and refer to the corresponding heavy chain immunoglobulin constant domain 3.

As used herein, the term “CH2-CH3”, as in (CH2-CH3)A and (CH2-CH3)B, thus refers to a polypeptide sequence comprising an immunoglobulin heavy chain constant domain 2 (CH2) and an immunoglobulin heavy chain constant domain 3 (CH3).

As used herein, the term “CL domain”, or “CL domain” can be used interchangeably and refer to the corresponding light chain immunoglobulin constant domain. Unless instructed otherwise, this term may thus encompass a CL domain of the kappa (κ or K) or lambda (λ) class of immunoglobulin light chains, including all known subtypes (e.g. λ1, λ2, λ3, and λ7). In particular, when the CL domain is of the kappa class, it may also be referred herein as a Cκ or CK or Ck domain.

As used herein, the terms “pair C (CH1/CL)”, or “paired C (CH1/CL)” “refers to one constant heavy chain domain 1 and one constant light chain domain (e.g., a kappa (κ or K) or lambda (λ) class of immunoglobulin light chains) bound to one another by covalent or non-covalent bonds, preferably non-covalent bonds; thus forming a heterodimer. Unless specified otherwise, when the constant chain domains forming the pair are not present on a same polypeptide chain, this term may thus encompass all possible combinations. Preferably, the corresponding CH1 and CL domains will thus be selected as complementary to each other, such that they form a stable pair C (CH1/CL).

Advantageously, when the binding protein comprises a plurality of paired C domains, such as one “pair C1 (CH1/CL)” and one “pair C2 (CH1/CL)”, each CH1 and CL domain forming the pairs will be selected so that they are formed between complementary CH1 and CL domains. Examples of complementary CH1 and CL domains have been previously described in the international patent applications WO2006064136 or WO2012089814 or WO2015197593A1.

Unless instructed otherwise, the terms “pair C1 (CH1/CL)” or “pair C2 (CH1/CL)” may refer to distinct constant pair domains (C1 and C2) formed by identical or distinct constant heavy 1 domains (CH1) and identical or distinct constant light chain domains (CL). Preferably, the terms “pair C1 (CH1/CL)” or “pair C2 (CH1/CL)” may refer to distinct constant pair domains (C1 and C2) formed by identical constant heavy 1 domains (CH1) and identical constant light chain domains (CL).

As used herein, the term “Fc region” or “fragment crystallizable region”, or alternatively “Fc portion”, encompasses all or parts of the “Fc domain”, which may thus include all or parts of an immunoglobulin hinge region (which natively bears a first binding site to FcγRs), a CH2 domain (which natively bears a second binding site to FcγRs), and a CH3 domain of an immunoglobulin (e.g. of an IgG, IgA or IgD immunoglobulin), and/or when applicable of a CH4 domain of an immunoglobulin (e.g. for IgM and IgE). Preferably, the Fc region includes all or parts of, at least, a CH2 domain and a CH3 domain, and optionally all or parts of an immunoglobulin hinge region. The term may thus refer to a molecule comprising the sequence of a non-antigen-binding fragment resulting from digestion of an antibody or produced by other means, whether in monomeric or multimeric form, and can contain the hinge region. The original immunoglobulin source of the native Fc is, in particular, of human origin and can be any of the immunoglobulins, although IgG1 are preferred. Native Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 13 depending on class (e.g., IgG, IgA, and IgE) or subclass (e.g., IgG1, IgG2, IgG3, IgGA1, and IgGA2). One example of a native Fc is a disulfide-bonded dimer resulting from papain digestion of an IgG. The term “native Fc” as used herein is generic to the monomeric, dimeric, and multimeric forms. Under that terminology, a “Fc region” may thus comprise or consist of CH2-CH3 (e.g., (CH2-CH3)A or (CH2-CH3)B or a binding pair thereof, and optionally all or part of an immunoglobulin hinge region, comprising a binding site to a human FcγR. Unless specified otherwise, the term “Fc region” may refer to either a native or variant Fc region.

The term “Fc variant” as used herein refers to a molecule or sequence that is modified from a native Fc but still comprises a binding site for the receptor, FcRn (neonatal Fc receptor). Exemplary Fc variants, and their interaction with the receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

The fragment crystallizable (Fc) regions (e.g., native or variant) according to the present disclosure retain a capacity to bind to a human Fc-γ receptor polypeptide (Fcγ) which generally occurs on native Fc regions through binding of the antibody Fc-hinge region. As a reference, overall structures of IgG1, IgG2, and IgG4 are similar with more than 90% sequence homology, the major differences residing in the hinge region and CH2 domain, which form primary binding sites to FcγRs. The hinge region also functions as a flexible linker between the Fab and Fc portion.

Fc regions having one or more amino acid modifications (e.g., substitutions, deletions, insertions) in one or more portions, which modifications increase the affinity and avidity of the variant Fc region for an FcγR (including activating and inhibitory FcγRs) are further considered as Fc regions. In some embodiments, said one or more amino acid modifications increase the affinity of the Fc region for FcγRIIIA and/or FcγRIIA. In another embodiment, the variant Fc region further specifically binds FcγRIIB with a lower affinity than does the Fc region of the reference parent antibody (e.g., an antibody having the same amino acid sequence as the antibody except for the one or more amino acid modifications in the Fc region). Hence, native and variant Fc regions considered herein generally comprise a domain (i.e., a CH2 domain) capable of binding to human CD16, e.g., a human Fc domain comprising N-linked glycosylation at amino acid residue N297 (according to EU numbering).

As used herein, the term “Fc-competent” thus refers to a binding protein that is capable of binding specifically to a FcγR, in particular of an activating FcγR, in particular to one selected from FcγRI (CD64a), FcγRIIa (CD32a), and FcγRIIIa (CD16a), and more particularly to FcγRIIIa (CD16a).

Alternatively, several modifications are reported to directly affect the binding to FcγRs, including mutation on residues 297 (according to EU numbering), or alternatively on residues 234 and 235 in the lower hinge region (according to the EU numbering system).

As used herein, the term “Fc-silent” refers to a binding protein with a Fc region, wherein the Fc region lacks a binding site to a FcγR (e.g., a Fc region lacking a CH2 domain with said binding site and hinge region with said binding site); in particular FcγRI, FcγRIIa, and FcγRIIIa, and more particularly to FcγRIIIa (CD16a).

As used herein, the term “variable”, as in “variable domain”, refers to certain portions of the relevant binding protein which differ extensively in sequence between and among antibodies and are used in the specific recognition and binding of a particular antibody for its particular target. However, the variability is not evenly distributed throughout the entire variable domains of antibodies. The variability is concentrated in three segments called complementarity determining regions (CDRs; i.e., CDR1, CDR2, and CDR3) also known as hypervariable regions, both in the light chain and the heavy chain variable domains. The more highly conserved portions of variable domains are called the framework (FR) regions or sequences.

As used herein, the term “VH domain”, or “VH domain” can be used interchangeably and refer to the corresponding heavy chain immunoglobulin variable domain.

As used herein, the term “VL domain”, or “VL domain” can be used interchangeably and refer to the corresponding light chain immunoglobulin variable domain.

When the VH or VL domains are associated to a first antigen-binding domain (ABD) or to a second antigen-binding domain, they may also be respectively referred herein as “VHT” and “VL1”, or “VH2” and “VL2”.

The terms “binding pair V (VHNL)”, “VHNL pair” or “(VHNL) pair” or “VL/VH pair” or “(VL/VH) pair” can be used interchangeably. Heavy chain and light chain variable domain can pair in parallel to form the antigen binding domains (ABDs). Each binding pair includes both a VH and a VL region. Unless instructed otherwise, these terms do not specify which immunoglobulin variable regions are VH or VL regions and which ABD will bind specifically the protein expressed on the surface of an immune effector cell or a target cell (e.g., NKp46 and CD123).

As used herein, the term “hypervariable region’ when used herein refers to the amino acid residues of an antibody that are responsible for antigen binding. This term may be substituted by the terms “Complementarity Determining Regions” or “CDRs”.

Thus, as used herein “Complementarity Determining Regions” or “CDRs” refer to amino acid sequences that together define the binding affinity and specificity of the natural Fv region of a native immunoglobulin binding site. The light and heavy chains of an immunoglobulin each have three CDRs, designated CDR-L1, CDR-L2, CDR-L3 and CDR-H1, CDR-H2, CDR-H3, respectively. A conventional antibody antigen-binding domain, therefore, includes six CDRs, comprising the CDR set from each of a heavy and a light chain variable region. Also, as used herein, “Framework Regions” (FRs) refer to amino acid sequences interposed between CDRs, i.e., to those portions of immunoglobulin light and heavy chain variable regions that are relatively conserved among different immunoglobulins in a single species. The light and heavy chains of an immunoglobulin each have four FRs, designated FR-L1, FR-L2, FR-L3, FR-L4, and FR-H1, FR-H2, FR-H3, FR-H4, respectively. Accordingly, the light chain variable domain may thus be designated as (FR-L1)-(CDR-L1)-(FR-L2)-(CDR-L2)-(FR-L3)-(CDR-L3)-(FR-L4) and the heavy chain variable domain may thus be designated as (FR-H1)-(CDR-H1)-(FR-H2)-(CDR-H2)-(FR-H3)-(CDR-H)-(FR4-H3).

The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). The numbering of amino acid residues in this region is performed by the method described in Kabat et al., supra. Accordingly, phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Optionally, CDRs are as defined by EU, Kabat, Chotia or IMGT numbering. Correspondences between those classifications are known in the Art, by reference to the IMGT®, or international ImMunoGeneTics information system® (CNRS and Montpellier University), and as further detailed in Lefranc (Biomolecules; 2014; 4, 1102-1139) and Dondelinger (Frontiers in Immunology; 2018; 9, 2278). CDRs may also be defined according to the Honegger-Pluckthun (“Honegger”) numbering scheme described in Honnegger and Pluckthun (2001), J. Mol. Biol., vol. 309(3):657-670.

Unless instructed otherwise, the numbering of residues will be considered herein by reference to the EU, Kabat, Chotia, IMGT, or Honegger-Pluckthun numbering convention. In case of conflict regarding the exact position of hypervariable regions within a reference sequence, the Kabat numbering convention will prevail. In case of conflict regarding the exact position of constant regions within a reference sequence, the EU numbering convention will prevail. Further,

As used herein, the term “cytotoxicity” refers to the quality of a compound, such as the multifunctional binding protein according to the present disclosure, to be toxic to tumoral cells. Cytotoxicity may be induced by different mechanisms of action and can thus be divided into cell-mediated cytotoxicity, apoptosis, antibody-dependent cell-mediated cytotoxicity (ADCC), antibody-dependent cellular phagocytosis (ADCP) or complement-dependent cytotoxicity (CDC).

As used herein, the term “antibody-dependent cell-mediated cytotoxicity” or “ADCC” refers to a mechanism of cell-mediated immune defence whereby an effector cell of the immune system actively lyses a target cell, whose membrane-surface antigens have been bound by specific antibodies or the multifunctional binding protein of the present disclosure.

As used herein, the terms “proliferative disorders”, “hyper-proliferative disorders” and/or “cancer” not only refer to solid tumors, such as cancers of the breast, respiratory tract, brain, reproductive organs, digestive tract, urinary tract, eye, liver, skin, head and neck, thyroid, parathyroid and their distant metastases, but also include blood cancers, including tumors of the hematopoietic and lymphoid tissues, such as lymphomas, myelomas, and leukemias. Leukemias include, but are not limited to acute myeloid leukemia, acute lymphoblastic leukemia, chronic lymphocytic leukemia, chronic myelogenous leukemia, and hairy cell leukemia.

As used herein, “Acute myelogenous leukemia (AML)” is a clonal disorder clinically presenting as increased proliferation of heterogeneous and undifferentiated myeloid blasts. Without wishing to be bound by the theory, the leukemic hierarchy is maintained by a small population of LSCs (Leukemic Stem Cells) (AML-LSCs), which have the distinct ability for self-renewal, and are able to differentiate into leukemic progenitors. These progenitors generate the large numbers of leukemic blasts readily detectable in patients at diagnosis and relapse, leading ultimately to mortality. AML-LSC have been commonly reported as quiescent cells, in contrast to rapidly dividing clonogenic progenitors.

Within the context of AML, the term “relapse” may in particular be defined as the reoccurrence of AML after complete remission. In that sense “complete remission” or “CR” may be defined as follows: normal values for neutrophil (>1.0*109/L), haemoglobin level of 10 g/dl and platelet count (>100*109/L) and independence from red cell transfusion; blast cells less than 5%, no clusters or collections of blasts, and absence of Auer rods on bone marrow examination; and normal maturation of blood cells (morphology; myelogramme) and absence of extramedullary leukemia.

As used herein, the term “refractory” means the cancer did not respond to treatment. In the context of AML, most patients achieve a remission (an absence of signs and symptoms) after initial treatment. However, some patients have residual leukemic cells in their marrow even after intensive treatment. Patients who have not achieved complete remission after two cycles of induction chemotherapy are usually diagnosed as having “refractory AML.”

As used herein, the term “blastic plasmacytoid dendritic cell neoplasm” or “BPDCN” is a rare hematologic malignancy of plasmacytoid dendritic cells. In 2016, the World Health Organization (WHO) designated BPDCN to be in its own category within the myeloid class of neoplasms. It is estimated that BPDCN constitutes about 0.44% of all hematological malignancies. BPDCN is an aggressive malignancy with features of cutaneous lymphoma and/or leukemia. Particularly in more advanced stages, the disease may also involve malignant plasmacytoid dendritic cell infiltrations in and thereby injury to the liver, spleen, lymph nodes, central nervous system, or other tissues. While the neoplasm occurs in individuals of all ages. In children, it afflicts males and females equally, but in adults it is far more common in males (about 75% of cases). Almost all cases of BPDCN present with CD123-overexpressing cells.

As used herein, “myelodysplastic syndromes” (“MDS”), formerly known as preleukemia, are a collection of hematological conditions that involve ineffective production (or dysplasia) of the myeloid class of blood cells. They represent a spectrum of clonal hematopoietic stem cell disorders characterized by progressive bone marrow failure and increased risk of progression to acute myeloid leukemia (“AML”, also known as “acute myelogenous leukemia”). The International Prognostic Scoring System (“IPSS”) is widely used to identify patients with high-risk features based on the severity of their cytopenias, bone marrow myeloblast percentage, and cytogenetic abnormalities.

As used herein, a “pharmaceutically acceptable carrier” is intended to include any and all carrier (such as any solvents, dispersion media, coatings, antibacterial and antifungal agents, isotonic and absorption delaying agents, and the like) which is compatible with pharmaceutical administration, in particular parenteral administration. The use of such media and agents for pharmaceutically active substances are known. Except insofar as any conventional media or agent is incompatible with the active compound, such media can be used in the compositions of the present disclosure. For example, preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In a non-exhaustive manner, pharmaceutically acceptable carriers include, but are not limited to, 0.01-0.1M (e.g., 0.05M) phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringeability exists. It should be stable under the conditions of manufacture and storage and will in an embodiment be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In certain embodiments, isotonic agents are included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

As used herein, and unless instructed otherwise, the term “at least one” may encompass “one or more”, or even “two or more” (or “a plurality”). For instance, it may encompass 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more than 100.

As used herein, and unless instructed otherwise, the term “less than . . . ” may encompass all values from 0 to the corresponding threshold, For instance, it may encompass less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or less than 100, when applicable.

As used herein, the term “cell” may encompass any prokaryotic cell or eukaryotic cell. Cell types which are particularly considered are those suitable for the production and/or engineering of recombinant antibodies, or fragments, or polypeptide chains thereof. In a non-exhaustive manner, such cells may be selected from the group consisting of: bacterial cells, yeast cells, mammalian cells, non-mammalian cells, insect cells, and plant cells.

The terms “host cell,” “host cell line,” and “host cell culture” as used herein, are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein. A host cell is any type of cellular system that can be used to generate binding proteins of the present disclosure. Host cells may thus include cultured cells, e.g., mammalian cultured cells, such as CHO cells, HEK cells, BHK cells, NS0 cells, SP2/0 cells, YO myeloma cells, P3X63 mouse myeloma cells, PER cells, PER.C6 cells or hybridoma cells, bacterial cells, yeast cells, insect cells, and plant cells, to name only a few.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present disclosure. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present disclosure, as well as positive and negative strand forms, and double-stranded forms. Isolated polynucleotides or nucleic acids according to the present disclosure further include such molecules produced synthetically. In addition, a polynucleotide or a nucleic acid may be or may include a regulatory element such as a promoter, ribosome binding site, or a transcription terminator.

The term “vector’ or “expression vector” is intended to mean the vehicle by which a nucleic acid, in particular a DNA or RNA sequence (e.g., a foreign gene), can be introduced into a host cell, so as to transform the host and promote expression (e.g., transcription and translation) of the introduced sequence.

### II. Binding Protein

Provided herein is a binding protein comprising a bispecific NK cell engager (NKCE) with a competent Fc domain that can bind to CD16a (FcγRIIIa) used in methods for treating leukemias and myelodysplastic syndromes. The NKCE of the disclosure functions as a trifunctional molecule that binds NKp46 and CD16a on the surface of the NK cells and CD123 on malignant cells. Co-engagement of a NK cell and a malignant cell by the CD123 NKCE of the present disclosure leads to the formation of an immunological synapse which induces NK-cell activation and degranulation.

In some embodiments, the binding protein is characterized in that it comprises a first antigen binding domain with binding specificity to CD123 and a second antigen binding domain with binding specificity to NKp46. In some embodiments, the binding protein comprises a third antigen binding domain that binds CD16. In some embodiments, the third antigen binding domain that binds CD16 is an antigen binding domain with binding specificity to CD16. In some embodiments, the third antigen binding domain is an Fc domain.

In some embodiments, the NKCE is characterized in that it comprises a first antigen binding domain with binding specificity to CD123 and a second antigen binding domain with binding specificity to NKp46, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that the first antigen binding domain with binding specificity comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

In some embodiments, the binding protein is characterized in that it comprises the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44.

In some embodiments, the binding protein is characterized in that it comprises the VH1 comprises an amino acid sequence of SEQ ID NO: 41, and wherein the VL1 comprises an amino acid sequence of SEQ ID NO: 43; or the VH1 comprises an amino acid sequence of SEQ ID NO: 42, and wherein the VL1 comprises an amino acid sequence of SEQ ID NO: 44.

In some embodiments, the binding protein comprises a second antigen binding domain with binding specificity to NKp46. In some embodiments, the second antigen binding domain comprises CDRs defined by Kabat numbering. In some embodiments, the second antigen binding domain comprises CDRs defined by IMGT numbering. In some embodiments, the second antigen binding domain comprises CDRs defined by Chothia numbering. In some embodiments, the second antigen binding domain comprises CDRs defined by Honegger numbering. In some embodiments, the second antigen binding domain comprises CDRs as defined in Table 1.

TABLE 1
Example Anti-NKp46 CDRs

| | Kabat | IMGT | Chothia | Honegger |
|---|---|---|---|---|
| CDR | RASQDISNYLN | QDISNY | SQDISNY | ASQDISNY |
| L1 | (SEQ ID NO: 27) | (SEQ ID NO: 99) | (SEQ ID NO: | (SEQ ID NO: 111) |
| | | | 105) | |
| CDR | YTSRLHS | YTS | YTS (SEQ ID | YTSRLHSGVPSR |
| L2 | (SEQ ID NO: 28) | (SEQ ID NO: 100) | NO: 106) | (SEQ ID NO: 112) |
| CDR | QQGNTRPWT | QQGNTRPWTF | GNTRPW | GNTRPW |
| L3 | (SEQ ID NO: 29) | (SEQ ID NO: 101) | (SEQ ID NO: | (SEQ ID NO: 107) |
| | | or | 107) | |
| | | QQGNTRPWT | | |
| | | (SEQ ID NO: 116) | | |
| CDR | DYVIN | GYTFSDYV | GYTFSDY | ASGYTFSDYV |
| H1 | (SEQ ID NO: 13) | (SEQ ID NO: 102) | (SEQ ID NO: | (SEQ ID NO: 113) |
| | | | 108) | |
| CDR | EIYPGSGTNYYNEKFKA | IYPGSGTN | PGSG (SEQ | IYPGSGTNYYNEKFKAK |
| H2 | (SEQ ID NO: 14) | (SEQ ID NO: 103) | ID NO: 109) | (SEQ ID NO: 114) |
| CDR | RGRYGLYAMDY | ARRGRYGLYAMDY | GRYGLYAMD | RGRYGLYAMD |
| H3 | (SEQ ID NO: 15) | SEQ ID NO: 104) | (SEQ ID NO: | (SEQ ID NO: 115) |
| | | | 110) | |

In some embodiments, the binding protein is characterized in that the second antigen binding domain with binding specificity to NKp46 comprises:
a. a second heavy chain variable domain (VH2) comprising a CDR-H1, H2, and H3 corresponding to the amino acid sequences of:
   i. SEQ ID NO: 13, 14, and 15, respectively;
   ii. SEQ ID NO: 16, 17, and 18, respectively;
   iii. SEQ ID NO: 19, 20, and 21, respectively;
   iv. SEQ ID NO: 22, 23, and 24, respectively; or
   v. SEQ ID NO: 16, 25, and 26, respectively; and
b. a second light chain variable domain (VL2) comprising a CDR-L1, L2, and L3 corresponding to the amino acid sequences of:
   i. SEQ ID NO: 27, 28, and 29, respectively;
   ii. SEQ ID NO: 30, 31, and 32, respectively;
   iii. SEQ ID NO: 33, 34, and 35, respectively;
   iv. SEQ ID NO: 36, 37, and 38, respectively; or
   v. SEQ ID NO: 39, 31, and 40, respectively.

In some embodiments, the binding protein is characterized in that:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53; or
b. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54
c. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60.

In some embodiments, the binding protein is characterized in that:
a. the VH2 comprises an amino acid sequence of SEQ ID NO: 45, and wherein the VL1 comprises an amino acid sequence of SEQ ID NO: 53; or
b. the VH2 comprises an amino acid sequence of SEQ ID NO: 46, and wherein the VL2 comprises an amino acid sequence of SEQ ID NO: 54
c. the VH2 comprises an amino acid sequence of SEQ ID NO: 47, and wherein the VL2 comprises an amino acid sequence of SEQ ID NO: 55;
d. the VH2 comprises an amino acid sequence of SEQ ID NO: 48, and wherein the VL2 comprises an amino acid sequence of SEQ ID NO: 56;
e. the VH2 comprises an amino acid sequence of SEQ ID NO: 49, and wherein the VL2 comprises an amino acid sequence of SEQ ID NO: 57;
f. the VH2 comprises an amino acid sequence of SEQ ID NO: 50, and wherein the VL2 comprises an amino acid sequence of SEQ ID NO: 58;
g. the VH2 comprises an amino acid sequence of SEQ ID NO: 51, and wherein the VL2 comprises an amino acid sequence of SEQ ID NO: 59; or
h. the VH2 comprises an amino acid sequence of SEQ ID NO: 52, and wherein the VL2 comprises an amino acid sequence of SEQ ID NO: 60.

In some embodiments, the binding protein is characterized in that the binding protein comprises three polypeptide chains (I), (II) and (III) that form two ABDs, as defined below:

V_{1A}-C_{1A}-Hinge₁-(C_{H}2-C_{H}3)_{A} (I)

V_{1B}-C_{1B}-Hinge₂-(C_{H}2-C_{H}3)_{B}-L₁-V_{2A}-C_{2A}-Hinge₃ (II)

V_{2B}-C_{2B} (III)

wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
   Hinge₁, Hinge₂ and Hinge₃ are identical or different and correspond to all or part of an immunoglobulin hinge region;
   (C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
   L₁ is an amino acid linker.

In some embodiments, the binding protein is characterized in that it comprises a C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO: 74;
Hinge₂ corresponds to the amino acid sequence of SEQ ID NO: 75;
Hinge₃ corresponds to the amino acid sequence of SEQ ID NO: 77;
L₁ corresponds to the amino acid sequence of SEQ ID NO: 76.

In some embodiments, the binding protein is characterized in that the residue N297 of the Fc region or variant thereof according to EU numbering comprises a N-linked glycosylation.

In some embodiments, the binding protein is characterized in that the all or part of the Fc region or variant thereof binds to a human CD16A (FcγRIII) polypeptide.

In some embodiments, the binding protein is characterized in that at least two polypeptide chains are linked by at least one disulfide bridge.

In some embodiments, the binding protein is characterized in that the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B}.

In some embodiments, the binding protein is characterized in that V_{1A} is V_{L1} and V_{1B} is V_{H1}.

In some embodiments, the binding protein is characterized in that V_{2A} is V_{H2} and V_{2B} is V_{L2}.

In some embodiments, the binding protein is characterized in that:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40.

In some embodiments, the binding protein is characterized in that:
(a) V_{H1} and V_{L1} corresponds to the amino acid sequences of SEQ ID NO: 41 and 43 respectively or corresponds to the amino acid sequences of SEQ ID NO: 42 and 44 respectively;
   and/or
(b) V_{H2} and V_{L2} corresponds to
   the amino acid sequences of SEQ ID NO: 45 and 53 respectively;
   the amino acid sequences of SEQ ID NO: 46 and 54 respectively;
   the amino acid sequences of SEQ ID NO: 47 and 55 respectively;
   the amino acid sequences of SEQ ID NO: 48 and 56 respectively;
   the amino acid sequences of SEQ ID NO: 49 and 57 respectively;
   the amino acid sequences of SEQ ID NO: 50 and 58 respectively;
   the amino acid sequences of SEQ ID NO: 51 and 59 respectively; or
   the amino acid sequences of SEQ ID NO: 52 and 60 respectively.

In some embodiments, the binding protein is characterized in that:
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(g) V₁ comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
(i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
(j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58.
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59;
(p) V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60.

In some embodiments, the binding protein is characterized in that:
polypeptide (I) consists of an amino acid sequence of SEQ ID NO: 64;
polypeptide (II) consists of an amino acid sequence of SEQ ID NO: 65; and
polypeptide (III) consists of an amino acid sequence of SEQ ID NO: 66.

### III. Methods of Use/Treatment

Provided herein are methods of treating or preventing a hematological disease or disorder in a subject in need thereof comprising administering to the subject a therapeutically effective amount of the CD123 NKCE of the present disclosure.

Provided herein is a method of treating or preventing a leukemia or a myelodysplastic syndrome or BPDCN in a subject in need thereof, the method comprising administering to the subject a binding protein comprising a first antigen binding domain with binding specificity to CD123 and a second antigen binding domain with binding specificity to NKp46.

In some embodiments, the subject in need thereof is an adult. In some embodiments, the adult is a subject over 17 years old. In some embodiments, the adult is a subject over 18 years old.

In some embodiments, the subject in need thereof is a pediatric subject. In some embodiments, the pediatric subject is between the ages of about 1 year old and about 17 years old. In some embodiments, the pediatric subject is between the ages of about 1 year old and about 11 years old.

A therapeutically effective dose of the NK cell engager disclosed herein may be the dose or amount sufficient to induce a “therapeutic response” in a subject, which as an improvement in at least one measure of a hematological disease or disorder (e.g., ALL, B-ALL, BPDCN or HR-MDS). In some embodiments, the therapeutically effective dose is expressed as μg per kg of the patient's body weight (μg/kg).

In some embodiments, the therapeutically effective dose is between about 1 μg/kg and about 6000 μg/kg. In some embodiments, the therapeutically effective dose is between about 1 μg/kg and about 10 μg/kg. In some embodiments, the therapeutically effective dose is between about 10 and about 100 μg/kg. In some embodiments, the therapeutically effective dose is between about 100 μg/kg and about 150 μg/kg. In some embodiments, the therapeutically effective dose is between about 150 μg/kg and about 200 μg/kg. In some embodiments, the therapeutically effective dose is between about 200 μg/kg and about 250 μg/kg. In some embodiments, the therapeutically effective dose is between about 250 μg/kg and about 300 μg/kg. In some embodiments, the therapeutically effective dose is between about 300 μg/kg and about 350 μg/kg. In some embodiments, the therapeutically effective dose is between about 350 μg/kg and about 400 μg/kg. In some embodiments, the therapeutically effective dose is between about 400 μg/kg and about 450 μg/kg. In some embodiments, the therapeutically effective dose is between about 450 μg/kg and about 500 μg/kg. In some embodiments, the therapeutically effective dose is between about 500 μg/kg and about 550 μg/kg. In some embodiments, the therapeutically effective dose is between about 550 μg/kg and about 600 μg/kg. In some embodiments, the therapeutically effective dose is between about 600 μg/kg and about 650 μg/kg. In some embodiments, the therapeutically effective dose is between about 650 μg/kg and about 700 μg/kg. In some embodiments, the therapeutically effective dose is between about 700 μg/kg and about 750 μg/kg. In some embodiments, the therapeutically effective dose is between about 750 μg/kg and about 800 μg/kg. In some embodiments, the therapeutically effective dose is between about 800 μg/kg and about 850 μg/kg. In some embodiments, the therapeutically effective dose is between about 850 μg/kg and about 900 μg/kg. In some embodiments, the therapeutically effective dose is between about 900 μg/kg and about 950 μg/kg. In some embodiments, the therapeutically effective dose is between about 950 μg/kg and about 1000 μg/kg. In some embodiments, the therapeutically effective dose is between about 1000 μg/kg and about 1300 μg/kg. In some embodiments, the therapeutically effective dose is between about 1300 μg/kg and about 1500 μg/kg. In some embodiments, the therapeutically effective dose is between about 1500 μg/kg and about 2000 μg/kg. In some embodiments, the therapeutically effective dose is between about 2000 μg/kg and about 2500 μg/kg. In some embodiments, the therapeutically effective dose is between about 2500 μg/kg and about 3000 μg/kg. In some embodiments, the therapeutically effective dose is between about 3000 μg/kg and about 4000 μg/kg. In some embodiments, the therapeutically effective dose is between about 4000 μg/kg and about 4500 μg/kg. In some embodiments, the therapeutically effective dose is between about 4500 μg/kg and about 5000 μg/kg. In some embodiments, the therapeutically effective dose is between about 5000 μg/kg and about 6000 μg/kg.

In some embodiments, the therapeutically effective dose is about 3 μg/kg. In some embodiments, the therapeutically effective dose is about 10 μg/kg. In some embodiments, the therapeutically effective dose is about 13 μg/kg. In some embodiments, the therapeutically effective dose is about 15 μg/kg. In some embodiments, the therapeutically effective dose is about 20 μg/kg. In some embodiments, the therapeutically effective dose is about 30 μg/kg. In some embodiments, the therapeutically effective dose is about 40 μg/kg. In some embodiments, the therapeutically effective dose is about 45 μg/kg. In some embodiments, the therapeutically effective dose is about 50 μg/kg. In some embodiments, the therapeutically effective dose is about 60 μg/kg. In some embodiments, the therapeutically effective dose is about 100 μg/kg. In some embodiments, the therapeutically effective dose is about 100 μg/kg. In some embodiments, the therapeutically effective dose is about 130 μg/kg. In some embodiments, the therapeutically effective dose is about 150 μg/kg. In some embodiments, the therapeutically effective dose is about 200 μg/kg. In some embodiments, the therapeutically effective dose is about 300 μg/kg. In some embodiments, the therapeutically effective dose is about 400 μg/kg. In some embodiments, the therapeutically effective dose is about 450 μg/kg. In some embodiments, the dose is about 500 μg/kg. In some embodiments, the therapeutically effective dose is about 600 μg/kg. In some embodiments, the therapeutically effective dose is about 750 μg/kg. In some embodiments, the therapeutically effective dose is about 1000 μg/kg. In some embodiments, the therapeutically effective dose is about 1300 μg/kg. In some embodiments, the therapeutically effective dose is about 1500 μg/kg. In some embodiments, the therapeutically effective dose is about 2000 μg/kg. In some embodiments, the therapeutically effective dose is about 3000 μg/kg. In some embodiments, the therapeutically effective dose is about 4000 μg/kg. In some embodiments, the therapeutically effective dose is about 4500 μg/kg. In some embodiments, the therapeutically effective dose is about 6000 μg/kg.

In certain embodiments, the present disclosure provides kits and methods for the treatment of diseases and disorders, e.g., hematological diseases or disorders in a mammalian subject in need of such treatment. In some embodiments, the hematological disease or disorder is acute myeloid leukemia (AML). In some embodiments, the AML is relapsed or refractory. In some embodiments, the hematological disease or disorder is blastic plasmocytic dendritic cell neoplasm (BPDCN). In some embodiments, the hematological disease or disorder is B cell acute lymphoblastic leukemia (B-ALL). In some embodiments, the hematological disease or disorder is high-risk myelodysplasia (HR-MDS). In some embodiments, the mammalian subject is a human patient. In some embodiments, the human patient is an adult patient. In some embodiments, the human patient is a pediatric patient.

The binding protein of the current disclosure are useful in a number of different applications. For example, in one embodiment, the subject binding proteins are useful for reducing or eliminating cells bearing an epitope recognized by a binding domain of the binding protein. In another embodiment, the subject binding proteins are effective in reducing the concentration of or eliminating soluble antigen in the circulation. In another embodiment, the subject binding proteins are effective as NK-cell engagers (NKCEs).

In another embodiment, the CD123 NKCEs are useful for the treatment of diseases or disorders associated with aberrant immune cells, e.g., myeloid cells, B cells. In some embodiments, the aberrant immune cells, e.g., myeloid cells or B cells, express CD123.

In some embodiments, the CD123 NKCEs of the present disclosure can be particularly useful in the treatment of a disease or disorder within the category of leukemias or myelodysplasias. In some embodiments, the leukemia is AML. In some embodiments, the disease or disorder is blastic plasmacytoid dendritic cell neoplasm (BPDCN). In some embodiments, the leukemia is B-ALL. In some embodiments, the myelodysplasia is high-risk myelodysplasia (HR-MDS).

### Acute Myeloid Leukemia (AML)

Newly diagnosed de novo AML patients capable of undergoing intensive induction therapy typically receive a cytosine arabinoside and anthracycline based induction therapy, followed by consolidation chemotherapy with cytarabine or other agents. Patients with AML are eligible to receive a liposomal formulation of daunorubicin/cytarabine. As the understanding of the molecular drivers of AML have improved, newer molecularly targeted therapies have emerged. Elderly patients (>75 years) or patients that have comorbidities that preclude aggressive chemotherapy induction are treated with less aggressive therapies that include venetoclax/hypomethylating agent combinations which provide better outcomes than prior single agent regimens.

Allogeneic stem cell transplantation is offered to patients with high-risk disease in first or subsequent remissions, provided that they have adequate organ function and a suitable source of stem cells. Although about 15% of patients >60 years of age and 40% of patients <60 years of age who receive intensive induction chemotherapy and/or allogeneic stem cell transplantation for AML are cured; AML patients with relapsed or refractory disease (r/r) following initial therapy exhibit a poor prognosis. Despite the development of several new agents for r/r AML, it is usually incurable. A short duration of remission (e.g., <6 months), adverse genetic factors, prior allogeneic transplantation, older age, and general health status are factors associated with worse survival outcomes in the setting of r/r AML, in part because these factors limit eligibility for further intensive therapy.

The relative 5-year survival rate from 2011 to 2017 for patients with AML was 29.5%. In 2022, 20,050 new cases of AML are estimated in the United States, with a median age at diagnosis of 68 years. Recent clinical trial data indicate a 3-year overall survival rate of only 50% to 60% in adults who have achieved a complete remission. Despite advances in understanding the pathophysiology of AML and recognizing its molecular heterogeneity, developing viable therapeutics for patients with AML is challenging.

### Blastic Plasmacytoid Dendritic Cell Neoplasm (BPDCN)

Blastic plasmacytoid dendritic cell neoplasm (BPDCN) is a rare, aggressive hematological malignancy arising from plasmacytoid dendritic cell precursors. In the past, several different nomenclatures have been used to describe BPDCN until 2008 when the World Health Organization (WHO) classification described BPDCN as an entity under the family of acute myeloid leukemia (AML) and related neoplasms. It was later given its own separate category under myeloid neoplasms in the WHO re-classification, reflecting its unique pathobiology. BPDCN generally presents with cutaneous lesions with or without bone marrow involvement, lympadenopathy, splenomegaly, cytopenias, and sometimes with extramedullary involvement. Almost all cases have associated CD123-overexpression by the malignant cells.

BPDCN is suggested by a biopsy of skin lesions which can reveal infiltration by medium-sized blast (i.e., immature) cells into the dermis while sparing the epidermis. These cells exhibit irregular nuclei, fine chromatin, and at least one small nucleolus. Such blast cells may also be observed in the circulation, bone marrow, or other tissues that may suggest BPDCN. However, the diagnosis of this disease requires determination that these cells are plasmacytoid dendritic cell blast cells rather than AML, T-cell lymphoblastic lymphoma, or aggressive NK-cell leukemia (NKL) blast cells.

There have been no controlled studies to define the optimal treatment for BPDCN. Studies on small numbers of individuals with the disease have found that the standard chemotherapy regimens used for the initial induction treatments of AML, ALL, and high-grade lymphoma give complete remission rates of 77%, 93%, and 80%, respectively, in childhood PBDN and 47%, 77%, and 53%, respectively, in adult PBDN. However, these remissions were short-lived: post-treatment mean times to relapse or death were 12 months for children and 6.8 months for adults. Given these poor remission and survival rates, other treatments have been added to the initial treatment regimens.

Currently, there is an approved treatment in the United States for the treatment of BPDCN. This treatment, Tagraxofusp-erzs, is a fusion protein consisting of IL-3 fused to diphtheria toxin. Other treatments, including CD123-specific CAR-T cells and venetoclax, are being investigated as potential treatments for BPDCN, however more research is required to find effective treatment regimens.

### B-Cell Acute Lymphoblastic Leukemia and High-Risk Myelodysplasia

Similar to AML, the most effective therapies for B-ALL and HR-MDS comprise intensive multi-agent chemotherapy and/or allogeneic stem cell transplantation; however, many patients with these diseases are elderly or have comorbidities that preclude these aggressive, highly toxic therapies. Furthermore, not all patients are cured with intensive therapy and/or allogeneic transplantation and there is a risk of treatment-associated toxicity.

Potential curative treatments for B-ALL require aggressive therapy that can be associated with significant toxicity. For B-ALL, a two-year course of intensive multi-agent chemotherapy and/or allogeneic stem cell transplantation can cure a substantial fraction of younger patients (age <40 years); however, outcomes for older adults are much worse. While salvage with CD19-directed chimeric antigen receptor-T cell (CAR-T) therapy (e.g., tisagenlecleucel) has led to durable remissions, it is not approved for treatment of patients over 25 years of age. Furthermore, CD123 is frequently expressed on B-ALL cells both at diagnosis and relapse, which indicates that CD123-directed therapy could be effective in this indication, even in the setting of disease that is resistant to CD19-directed therapy, such as tisagenlecleucel or blinatumomab, a T-cell engaging antibody targeting CD18. For HR-MDS, hypomethylating agents have been the predominant treatment, while other treatment options are limited. Allogeneic stem cell transplantation provides the only known cures for HR-MDS, but is not always a viable treatment option, given patient age and co-morbidities.

### IV. Pharmaceutical Compositions and Administration Thereof

Methods of preparing and administering the CD123 NKCE of the present disclosure to a subject are well known to or are readily determined by those skilled in the art. The route of administration of the NKCE of the present disclosure may be oral, parenteral, by inhalation, or topical. The term parenteral as used herein includes intravenous, intraarterial, intraperitoneal, intramuscular, subcutaneous, rectal or vaginal administration. While all these forms of administration are clearly contemplated as being within the scope of the present disclosure, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. In some embodiments, the NKCE can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Aqueous carriers include water, alcoholic/aqueous solutions, emulsions or suspensions, including saline and buffered media. In the compositions and methods of the current disclosure, pharmaceutically acceptable carriers include, but are not limited to, 0.01-0.1 M, e.g., 0.05 M phosphate buffer, or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will typically be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

In many cases, isotonic agents will be included, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an NKCE by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, exemplary methods of preparation include vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit. Such articles of manufacture will typically have labels or package inserts indicating that the associated compositions are useful for treating a subject suffering from or predisposed to autoimmune or neoplastic disorders.

Effective doses of the compositions of the present disclosure, for the treatment of the above-described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

CD123 NKCE proteins of the present disclosure can be administered on multiple occasions. Intervals between single dosages can be weekly, monthly or yearly. Intervals can also be irregular as indicated by measuring blood levels of Fc domain variant or antigen in the patient. In some methods, dosage is adjusted to achieve a plasma modified binding polypeptide concentration of about 1-1000 μg/ml and in some methods about 25-300 μg/ml. Alternatively, Fc domain variants can be administered as a sustained release formulation, in which case less frequent administration is required. For antibodies, dosage and frequency vary depending on the half-life of the antibody in the patient. In general, humanized antibodies show the longest half-life, followed by chimeric antibodies and nonhuman antibodies.

The dosage and frequency of administration can vary depending on whether the treatment is prophylactic or therapeutic. In prophylactic applications, compositions containing the present polypeptides or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from about 0.001 to about 25 mg/kg per dose, especially about 0.003 to about 6.0 mg/kg per dose. A relatively low dosage is administered at relatively infrequent intervals over a long period of time. Some patients continue to receive treatment for the rest of their lives. In therapeutic applications, a relatively high dosage (e.g., from about 1 to 400 mg/kg of antibody per dose, with dosages of from about 5 to 25 mg being more commonly used for radioimmunoconjugates and higher doses for cytotoxin-drug modified antibodies) at relatively short intervals is sometimes required until progression of the disease is reduced or terminated, or until the patient shows partial or complete amelioration of disease symptoms. Thereafter, the patient can be administered a prophylactic regime.

NKCEs of the present disclosure can optionally be administered in combination with other agents that are effective in treating the disorder or condition in need of treatment (e.g., prophylactic or therapeutic). Effective single treatment dosages (i.e., therapeutically effective amounts) of 90Y-labeled modified antibodies of the current disclosure range from between about 5 and about 75 mCi, such as between about 10 and about 40 mCi. Effective single treatment non-marrow ablative dosages of 131I-modified antibodies range from between about 5 and about 70 mCi, or between about 5 and about 40 mCi. Effective single treatment ablative dosages (i.e., may require autologous bone marrow transplantation) of 131I-labeled antibodies range from between about 30 and about 600 mCi, such as between about 50 and less than about 500 mCi. In conjunction with a chimeric antibody, owing to the longer circulating half-life vis-a-vis murine antibodies, an effective single treatment non-marrow ablative dosage of iodine-131 labeled chimeric antibodies range from between about 5 and about 40 mCi, such as less than about 30 mCi. Imaging criteria for, e.g., the 111In label, are typically less than about 5 mCi.

While the NKCEs may be administered as described immediately above, it must be emphasized that in other embodiments the polypeptide may be administered to otherwise healthy patients as a first line therapy. In such embodiments, the NKCEs may be administered to patients having normal or average red marrow reserves and/or to patients that have not, and are not, undergoing treatment. As used herein, the administration of the polypeptides in conjunction or combination with an adjunct therapy means the sequential, simultaneous, coextensive, concurrent, concomitant, or contemporaneous administration or application of the therapy and the disclosed antibodies. Those skilled in the art will appreciate that the administration or application of the various components of the combined therapeutic regimen may be timed to enhance the overall effectiveness of the treatment.

As previously discussed, the NKCEs of the present disclosure, antibodies, therapeutic polypeptides, or NKCE fusion polypeptides thereof, may be administered in a pharmaceutically effective amount for the in vivo treatment of mammalian disorders. In this regard, it will be appreciated that the disclosed NKCEs will be formulated to facilitate administration and promote stability of the active agent.

A pharmaceutical composition in accordance with the present disclosure can comprise a pharmaceutically acceptable, non-toxic, sterile carrier such as physiological saline, nontoxic buffers, preservatives and the like. For the purposes of the instant application, a pharmaceutically effective amount of the CD123 NKCE, conjugated or unconjugated to a therapeutic agent, shall be held to mean an amount sufficient to achieve effective binding to an antigen and to achieve a benefit, e.g., to ameliorate symptoms of a disease or disorder or to detect a substance or a cell. In the case of plasma cells, the polypeptide can interact with selected antigens on immunoreactive cells and provide for an increase in the death of those cells. Of course, the pharmaceutical compositions of the present disclosure may be administered in single or multiple doses to provide for a pharmaceutically effective amount of the modified binding polypeptide.

In keeping with the scope of the present disclosure, the CD123 NKCEs of the disclosure may be administered to a human or other animal in accordance with the aforementioned methods of treatment in an amount sufficient to produce a therapeutic or prophylactic effect. The CD123 NKCEs of the disclosure can be administered to such human or other animal in a conventional dosage form prepared by combining the antibody of the disclosure with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of binding polypeptides described in the current disclosure may prove to be particularly effective.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present invention has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present invention. All such modifications are intended to be within the scope of the claims appended hereto. Having now described certain embodiments in detail, the same will be more clearly understood by reference to the following examples, which are included for purposes of illustration only and are not intended to be limiting.

### SEQUENCE LISTING

In the protein sequences notation used herein, the left-hand direction is the amino terminal direction (the “N terminus” or “N-term”) and the right-hand direction is the carboxyl-terminal direction (the “C terminus” or “C-term”), in accordance with standard usage and convention.

| SEQ | Name | Sequence |
|---|---|---|
| 1 | V_{H1} CDR-H1 | GYSFTDYYMK |
| | Anti-CD123 | |
| | CD123-1 | |
| 2 | V_{H1} CDR-H2 | DIIPSSGATF |
| | Anti-CD123 | |
| | CD123-1 | |
| 3 | V_{H1} CDR-H3 | SHLLRASWFAY |
| | Anti-CD123 | |
| | CD123-1 | |
| 4 | Anti-CD123 | GFTFSHYN |
| | CD123-2 | |
| 5 | V_{H1} CDR-H2 | ITYDDHST |
| | Anti-CD123 | |
| | CD123-2 | |
| 6 | V_{H1} CDR-H3 | ARLVNYAFAY |
| | Anti-CD123 | |
| | CD123-2 | |
| 7 | V_{L1} CDR-L1 | ESSQSLLSSGNQKNYLT |
| | Anti-CD123 | |
| | CD123-1 | |
| 8 | V_{L1} CDR-L2 | WASTRES |
| | Anti-CD123 | |
| | CD123-1 | |
| 9 | V_{L1} CDR-L3 | QNDYSYPYT |
| | Anti-CD123 | |
| | CD123-1 | |
| 10 | V_{L1} CDR-L1 | QTVGNN |
| | Anti-CD123 | |
| | CD123-2 | |
| 11 | V_{L1} CDR-L2 | YAS |
| | Anti-CD123 | |
| | CD123-2 | |
| 12 | V_{L1} CDR-L3 | QRMYNSPT |
| | Anti-CD123 | |
| | CD123-2 | |
| 13 | V_{H2} CDR-H1 | DYVIN |
| | Anti-NKp46 | |
| | NKp46-1 | |
| 14 | V_{H2} CDR-H2 | EIYPGSGTNYYNEKFKA |
| | Anti-NKp46 | |
| | 3D9 and NKp46-1 | |
| 15 | V_{H2} CDR-H3 | RGRYGLYAMDY |
| | Anti-NKp46 | |
| | 3D9 and NKp46-1 | |
| 16 | V_{H2} CDR-H1 | SDYAWN |
| | Anti-NKp46 | |
| | NKp46-2 | |
| | 13G4 | |
| 17 | V_{H2} CDR-H2 | YITYSGSTSYNPSLES |
| | Anti-NKp46 | |
| | NKp46-2 | |
| 18 | V_{H2} CDR-H3 | GGYYGSSWGVFAY |
| | Anti-NKp46 | |
| | NKp46-2 | |
| 19 | V_{H2} CDR-H1 | EYTMH |
| | Anti-NKp46 | |
| | NKp46-3 | |
| 20 | V_{H2} CDR-H2 | GISPNIGGTSYNQKFKG |
| | Anti-NKp46 | |
| | NKp46-3 | |
| 21 | V_{H2} CDR-H3 | RGGSFDY |
| | Anti-NKp46 | |
| | NKp46-3 | |
| 22 | V_{H2} CDR-H1 | SFTMH |
| | Anti-NKp46 | |
| | NKp46-4 | |
| 23 | V_{H2} CDR-H2 | YINPSSGYTEYNQKFKD |
| | Anti-NKp46 | |
| | NKp46-4 | |
| 24 | V_{H2} CDR-H3 | GSSRGFDY |
| | Anti-NKp46 | |
| | NKp46-4 | |
| 25 | V_{H2} CDR-H2 | YITYSGSTNYNPSLKS |
| | Anti-NKp46 | |
| | 13G4 | |
| 26 | V_{H2} CDR-H3 | CWDYALYAMDC |
| | Anti-NKp46 | |
| | 13G4 | |
| 27 | V_{L2} CDR-H1 | RASQDISNYLN |
| | Anti-NKp46 | |
| | 3D9 and NKp46-1 | |
| 28 | V_{L2} CDR-H2 | YTSRLHS |
| | Anti-NKp46 | |
| | 3D9 and NKp46-1 | |
| 29 | V_{L2} CDR-H3 | QQGNTRPWT |
| | Anti-NKp46 | |
| | 3D9 and NKp46-1 | |
| 30 | V_{L2} CDR-H1 | RVSENIYSYLA |
| | Anti-NKp46 | |
| | NKp46-2 | |
| 31 | V_{L2} CDR-H2 | NAKTLAE |
| | Anti-NKp46 | |
| | NKp46-2 | |
| | 13G4 | |
| 32 | V_{L2} CDR-H3 | QHHYGTPWT |
| | Anti-NKp46 | |
| | NKp46-2 | |
| 33 | V_{L2} CDR-H1 | RASQSISDYLH |
| | Anti-NKp46 | |
| | NKp46-3 | |
| 34 | V_{L2} CDR-H2 | YASQSIS |
| | Anti-NKp46 | |
| | NKp46-3 | |
| 35 | V_{L2} CDR-H3 | QNGHSFPLT |
| | Anti-NKp46 | |
| | NKp46-3 | |
| 36 | V_{L2} CDR-H1 | RASENIYSNLA |
| | Anti-NKp46 | |
| | NKp46-4 | |
| 37 | V_{L2} CDR-H2 | AATNLAD |
| | Anti-NKp46 | |
| | NKp46-4 | |
| 38 | V_{L2} CDR-H3 | QHFWGTPRT |
| | Anti-NKp46 | |
| | NKp46-4 | |
| 39 | V_{L2} CDR-H2 | RTSENIYSYLA |
| | Anti-NKp46 | |
| | 13G4 | |
| 40 | V_{L2} CDR-H3 | QHHYDTPLT |
| | Anti-NKp46 | |
| | 13G4 | |
| 41 | VH-full length- | EVQLVQSGAEVKKPGESLKISCKGSGYSFTDYYMKWARQM |
| | anti-CD123 | PGKGLEWMGDIIPSSGATFYNQKFKGQVTISADKSISTTYLQ |
| | | WSSLKASDTAMYYCARSHLLRASWFAYWGQGTMVTVSS |
| 42 | VH-full length- | EVQLVESGGGLVQPGRSLKLSCAASGFTFSHYNMAWVRQA |
| | anti-CD123 | PKKGLEWVATITYDDHSTYYRDSVKGRFTISRDTAKSTLYL |
| | | QMDSLRSEDTATYYCARLVNYAFAYWGQGTLVTVSS |
| 43 | VL-full length- | DIVMTQSPDSLAVSLGERATINCESSQSLLSSGNQKNYLTWY |
| | anti-CD123 | QQKPGQPPKPLIYWASTRESGVPDRFSGSGSGTDFTLTISSLQ |
| | | AEDVAVYYCQNDYSYPYTFGQGTKLEIK |
| 44 | VL-full length- | NIVMTQSPKSMSISVGDRVTMNCKASQTVGNNIAWYQQKP |
| | anti-CD123 | GLSPQLLIDYASNRYTGVPNRFTGGGYGTDFILTINSVQAED |
| | | AAFYYCQRMYNSPTFGGGTKLELK |
| 45 | VH-full length- | QVQLVQSGAEVKKPGSSVKVSCKASGYTFSDYVINWVRQA |
| | anti-NKp46 | PGQGLEWMGEIYPGSGTNYYNEKFKAKATITADKSTSTAYM |
| | | ELSSLRSEDTAVYYCARRGRYGLYAMDYWGQGTTVTVSS |
| 46 | VH-full length- | QVQLQQSGPELVKPGASVKMSCKASGYTFTDYVINWGKQR |
| | anti-NKp46 | SGQGLEWIGEIYPGSGTNYYNEKFKAKATLTADKSSNIAYM |
| | NKp46-1 | QLSSLTSEDSAVYFCARRGRYGLYAMDYWGQGTSVTVSS |
| 47 | VH-full length- | EVQLQESGPGLVKPSQSLSLTCTVTGYSITSDYAWNWIRQFP |
| | anti-NKp46 | GNKLEWMGYITYSGSTSYNPSLESRISITRDTSTNQFFLQLNS |
| | NKp46-2 | VTTEDTATYYCARGGYYGSSWGVFAYWGQGTLVTVSA |
| 48 | VH-full length- | EVQLQQSGPELVKPGASVKISCKTSGYTFTEYTMHWVKQSH |
| | anti-NKp46 | GKSLEWIGGISPNIGGTSYNQKFKGKATLTVDKSSSTAYMEL |
| | NKp46-3 | RSLTSEDSAVYYCARRGGSFDYWGQGTTLTVSS |
| 49 | VH-full length- | QVQLQQSAVELARPGASVKMSCKASGYTFTSFTMHWVKQR |
| | anti-NKp46 | PGQGLEWIGYINPSSGYTEYNQKFKDKTTLTADKSSSTAYM |
| | NKp46-4 | QLDSLTSDDSAVYYCVRGSSRGFDYWGQGTLVTVSA |
| 50 | VH-full length- | QVQLQESGPGLVKPSQTLSLTCTVSGYSISSDYAWNWIRQPP |
| | anti-NKp46 | GKGLEWIGYITYSGSTSYNPSLESRVTISRDTSKNQFSLKLSS |
| | 10B8 | VTAADTAVYYCARGGYYGSSWGVFAYWGQGTLVTVSS |
| 51 | VH-full length- | QVQLVQSGAEVKKPGASVKVSCKASGYTFTSFTMHWVRQA |
| | anti-NKp46 | PGQGLEWIGYINPSSGYTEYNQKFKDRVTITADKSTSTAYME |
| | 12E12 | LSSLRSEDTAVYYCVRGSSRGFDYWGQGTLVTVSS |
| 52 | VH-full length- | QVQLQESGPGLVKPSQTLSLTCTVSGYSISSDYAWNWIRQPP |
| | anti-NKp46 | GKGLEWIGYITYSGSTNYNPSLKSRVTISRDTSKNQFSLKLSS |
| | 13G4 | VTAADTAVYYCARCWDYALYAMDCWGQGTTVTVSS |
| 53 | VL-full length- | DIQMTQSPSSLSASVGDRVTITCRASQDISNYLNWYQQKPGK |
| | anti-NKp46 | APKLLIYYTSRLHSGVPSRFSGSGSGTDFTFTISSLQPEDIATYF |
| | 3D9 | CQQGNTRPWTFGGGTKVEIK |
| 54 | VL-full length- | DIQMTQTTSSLSASLGDRVTISCRASQDISNYLNWYQQKPDG |
| | anti-NKp46 | TVKLLIYYTSRLHSGVPSRFSGSGSGTDYSLTINNLEQEDIAT |
| | NKp46-1 | YFCQQGNTRPWTFGGGTKLEIK |
| 55 | VL-full length- | DIQMTQSPASLSASVGETVTITCRVSENIYSYLAWYQQKQG |
| | anti-NKp46 | KSPQLLVYNAKTLAEGVPSRFSGSGSGTQFSLKINSLQPEDF |
| | NKp46-2 | GSYYCQHHYGTPWTFGGGTKLEIK |
| 56 | VL-full length- | DIVMTQSPATLSVTPGDRVSLSCRASQSISDYLHWYQQKSH |
| | anti-NKp46 | ESPRLLIKYASQSISGIPSRFSGSGSGSDFTLSINSVEPEDVGV |
| | NKp46-3 | YYCQNGHSFPLTFGAGTKLELK |
| 57 | VL.full length- | DIQMIQSPASLSVSVGETVTITCRASENIYSNLAWFQQKQGK |
| | anti-NKp46 | SPQLLVYAATNLADGVPSRFSGSGSGTQYSLKINSLQSEDFG |
| | NKp46-4 | IYYCQHFWGTPRTFGGGTKLEIK |
| 58 | VL-full length- | DIQMTQSPSSLSASVGDRVTITCRVSENIYSYLAWYQQKPGK |
| | anti-NKp46 | APKLLVYNAKTLAEGVPSRFSGSGSGTDFTLTISSLQPEDFAT |
| | 10B8 | YYCQHHYGTPWTFGGGTKVEIK |
| 59 | VL-full length- | DIQMTQSPSSLSASVGDRVTITCRASENIYSNLAWFQQKPGK |
| | anti-NKp46 | APKLLVYAATNLADGVPSRFSGSGSGTDYTLTISSLQPEDFA |
| | 12E12 | TYYCQHFWGTPRTFGGGTKVEIK |
| 60 | VL-full length- | DIQMTQSPSSLSASVGDRVTITCRTSENIYSYLAWCQQKPGK |
| | anti-NKp46 | APKLLIYNAKTLAEGVPSRFSGSGSGTDFTLTISSLQPEDFAT |
| | 13G4 | YYCQHHYDTPLTFGQGTKLEIK |
| 61 | Polypeptide chain | MSVPTQVLGLLLLWLTDARCDIVMTQSPDSLAVSLGERATI |
| | (I) | NCESSQSLLSSGNQKNYLTWYQQKPGQPPKPLIYWASTRES |
| | | GVPDRFSGSGSGTDFTLTISSLQAEDVAVYYCQNDYSYPYTF |
| | | GQGTKLEIKRTVAAPSVFIFPPSDEQLKSGTASVVCLLNNFY |
| | | PREAKVQWKVDNALQSGNSQESVTEQDSKDSTYSLSSTLTL |
| | | SKADYEKHKVYACEVTHQGLSSPVTKSFNRGECDKTHTCPP |
| | | CPAPELLGGPSVFLFPPKPKDTLMISRTPEVTCVVVDVSHED |
| | | PEVKFNWYVDGVEVHNAKTKPREEQYNSTYRVVSVLTVLH |
| | | QDWLNGKEYKCKVSNKALPAPIEKTISKAKGQPREPQVYTL |
| | | PPSREEMTKNQVSLTCLVKGFYPSDIAVEWESNGQPENNYK |
| | | TTPPVLDSDGSFFLYSKLTVDKSRWQQGNVFSCSVMHEALH |
| | | NHYTQKSLSLSPGK |
| 62 | Polypeptide chain | MEWSWVFLFFLSVTTGVHSEVQLVQSGAEVKKPGESLKISC |
| | (II) | KGSGYSFTDYYMKWARQMPGKGLEWMGDIIPSSGATFYNQ |
| | | KFKGQVTISADKSISTTYLQWSSLKASDTAMYYCARSHLLR |
| | | ASWFAYWGQGTMVTVSSASTKGPSVFPLAPSSKSTSGGTAA |
| | | LGCLVKDYFPEPVTVSWNSGALTSGVHTFPAVLQSSGLYSL |
| | | SSVVTVPSSSLGTQTYICNVNHKPSNTKVDKRVEPKSCDKT |
| | | HTCPPCPAPELLGGPSVFLFPPKPKDTLMISRTPEVTCVVVD |
| | | VSHEDPEVKFNWYVDGVEVHNAKTKPREEQYNSTYRVVSV |
| | | LTVLHQDWLNGKEYKCKVSNKALPAPIEKTISKAKGQPREP |
| | | QVYTLPPSREEMTKNQVSLTCLVKGFYPSDIAVEWESNGQP |
| | | ENNYKTTPPVLDSDGSFFLYSKLTVDKSRWQQGNVFSCSVM |
| | | HEALHNHYTQKSLSLSPGSTGSQVQLVQSGAEVKKPGSSVK |
| | | VSCKASGYTFSDYVINWVRQAPGQGLEWMGEIYPGSGTNY |
| | | YNEKFKAKATITADKSTSTAYMELSSLRSEDTAVYYCARRG |
| | | RYGLYAMDYWGQGTTVTVSSASTKGPSVFPLAPSSKSTSGG |
| | | TAALGCLVKDYFPEPVTVSWNSGALTSGVHTFPAVLQSSGL |
| | | YSLSSVVTVPSSSLGTQTYICNVNHKPSNTKVDKRVEPKSCD |
| | | KTHS |
| 63 | Polypeptide chain | MSVPTQVLGLLLLWLTDARCDIQMTQSPSSLSASVGDRVTIT |
| | (III) | CRASQDISNYLNWYQQKPGKAPKLLIYYTSRLHSGVPSRFS |
| | | GSGSGTDFTFTISSLQPEDIATYFCQQGNTRPWTFGGGTKVEI |
| | | KRTVAAPSVFIFPPSDEQLKSGTASVVCLLNNFYPREAKVQ |
| | | WKVDNALQSGNSQESVTEQDSKDSTYSLSSTLTLSKADYEK |
| | | HKVYACEVTHQGLSSPVTKSFNRGEC |
| 64 | F25 polypeptide | DIVMTQSPDSLAVSLGERATINCESSQSLLSSGNQKNYLTWY |
| | chain (I) | QQKPGQPPKPLIYWASTRESGVPDRFSGSGSGTDFTLTISSLQ |
| | Cleaved (without | AEDVAVYYCQNDYSYPYTFGQGTKLEIKRTVAAPSVFIFPPS |
| | leader peptide) | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGECDKTHTCPPCPAPELLGGPSVFLFPPKPKDTL |
| | | MISRTPEVTCVVVDVSHEDPEVKFNWYVDGVEVHNAKTKP |
| | | REEQYNSTYRVVSVLTVLHQDWLNGKEYKCKVSNKALPAP |
| | | IEKTISKAKGQPREPQVYTLPPSREEMTKNQVSLTCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| 65 | F25 polypeptide | EVQLVQSGAEVKKPGESLKISCKGSGYSFTDYYMKWARQM |
| | chain (II) | PGKGLEWMGDIIPSSGATFYNQKFKGQVTISADKSISTTYLQ |
| | Cleaved (without | WSSLKASDTAMYYCARSHLLRASWFAYWGQGTMVTVSSA |
| | leader peptide) | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGST |
| | | GSQVQLVQSGAEVKKPGSSVKVSCKASGYTFSDYVINWVR |
| | | QAPGQGLEWMGEIYPGSGTNYYNEKFKAKATITADKSTSTA |
| | | YMELSSLRSEDTAVYYCARRGRYGLYAMDYWGQGTTVTV |
| | | SSASTKGPSVFPLAPSSKSTSGGTAALGCLVKDYFPEPVTVS |
| | | WNSGALTSGVHTFPAVLQSSGLYSLSSVVTVPSSSLGTQTYI |
| | | CNVNHKPSNTKVDKRVEPKSCDKTHS |
| 66 | F25 polypeptide | DIQMTQSPSSLSASVGDRVTITCRASQDISNYLNWYQQKPGK |
| | chain (III) | APKLLIYYTSRLHSGVPSRFSGSGSGTDFTFTISSLQPEDIATY |
| | Cleaved (without | FCQQGNTRPWTFGGGTKVEIKRTVAAPSVFIFPPSDEQLKSG |
| | leader peptide) | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 67 | Cκ | RTVAAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC |
| 68 | C_{H}1 | ASTKGPSVFPLAPSSKSTSGGTAALGCLVKDYFPEPVTVSWN |
| | | SGALTSGVHTFPAVLQSSGLYSLSSVVTVPSSSLGTQTYICN |
| | | VNHKPSNTKVDKRV |
| 69 | (C_{H}2-C_{H}3)_{A} | APELLGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALPAPIEKTISKAKGQPREPQVYTLP |
| | | PSREEMTKNQVSLTCLVKGFYPSDIAVEWESNGQPENNYKT |
| | | TPPVLDSDGSFFLYSKLTVDKSRWQQGNVFSCSVMHEALHN |
| | | HYTQKSLSLSPGK |
| 70 | (C_{H}2-C_{H}3)_{B} | APELLGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALPAPIEKTISKAKGQPREPQVYTLP |
| | | PSREEMTKNQVSLTCLVKGFYPSDIAVEWESNGQPENNYKT |
| | | TPPVLDSDGSFFLYSKLTVDKSRWQQGNVFSCSVMHEALHN |
| | | HYTQKSLSLSPG |
| 71 | C_{H}2 | APELLGGPSVFLFPPKPKDTLMISRTPEVTCVVVDVSHEDPE |
| | | VKFNWYVDGVEVHNAKTKPREEQYNSTYRVVSVLTVLHQ |
| | | DWLNGKEYKCKVSNKALPAPIEKTISKAK |
| 72 | C_{H}3 of | GQPREPQVYTLPPSREEMTKNQVSLTCLVKGFYPSDIAVEW |
| | (C_{H}2-C_{H}3)_{A} | ESNGQPENNYKTTPPVLDSDGSFFLYSKLTVDKSRWQQGNV |
| | | FSCSVMHEALHNHYTQKSLSLSPGK |
| 73 | C-term truncated | GQPREPQVYTLPPSREEMTKNQVSLTCLVKGFYPSDIAVEW |
| | C_{H}3 of | ESNGQPENNYKTTPPVLDSDGSFFLYSKLTVDKSRWQQGNV |
| | (CH₂-C_{H}3)_{B} | FSCSVMHEALHNHYTQKSLSLSPG |
| 74 | Hinge₁ | DKTHTCPPCP |
| 75 | Hinge₂ | EPKSCDKTHTCPPCP |
| 76 | Linker₁ | STGS |
| 77 | Hinge₃ | EPKSCDKTHS |
| 78 | Hinge_{ALT1} | EPKSCDKTH |
| 79 | Hinge_{ALT2} | EPKSCDKTHT |
| 80 | Human NKp46 | MSSTLPALLCVGLCLSQRISAQQQTLPKPFIWAEPHFMVPKE |
| | extracellular | KQVTICCQGNYGAVEYQLHFEGSLFAVDRPKPPERINKVKF |
| | domain (ECD) | YIPDMNSRMAGQYSCIYRVGELWSEPSNLLDLVVTEMYDTP |
| | | TLSVHPGPEVISGEKVTFYCRLDTATSMFLLLKEGRSSHVQR |
| | | GYGKVQAEFPLGPVTTAHRGTYRCFGSYNNHAWSFPSEPV |
| | | KLLVTGDIENTSLAPEDPTFPADTWGTYLLTTETGLQKDHAL |
| | | WDHTAQN |
| 81 | Cynomolgus | MSSTLRALLCLGLCLSQRISAPKQTLPKPIIRAESTYMVPKEK |
| | NKp46-FlagM2 | QATLCCQGSYGAVEYQLHFEGSLFAVERPKPPERINGVKFHI |
| | extracellular | PDMNSRKAGRYSCIYRVGELWSERSDLLDLVVTEMYDTPTL |
| | domain (ECD) | SVHPGPEVTSGEKVTFYCRLDTATSMFLLLKEGRSRDVQRS |
| | | YGKVQAEFPMGPVTTAHRGSYRCFGSYNNYAWSFPSEPVK |
| | | LLVTGDIENTSLAPTDPTFPDSWDTCLLTRETGLQKDLALW |
| | | DHTAQNDYKDDDDK |
| 82 | Human CD123 | TKEDPNPPITNLRMKAKAQQLTWDLNRNVTDIECVKDADY |
| | extracellular | SMPAVNNSYCQFGAISLCEVTNYTVRVANPPFSTWILFPENS |
| | domain (ECD) | GKPWAGAENLTCWIHDVDFLSCSWAVGPGAPADVQYDLY |
| | | LNVANRRQQYECLHYKTDAQGTRIGCRFDDISRLSSGSQSS |
| | | HILVRGRSAAFGIPCTDKFVVFSQIEILTPPNMTAKCNKTHSF |
| | | MHWKMRSHFNRKFRYELQIQKRMQPVITEQVRDRTSFQLL |
| | | NPGTYTVQIRARERVYEFLSAWSTPQRFECDQEEGANTRAW |
| | | R |
| 83 | Human Fc- | MWQLLLPTALLLLVSAGMRTEDLPKAVVFLEPQWYRVLEK |
| | gamma-receptor | DSVTLKCQGAYSPEDNSTQWFHNESLISSQASSYFIDAATVD |
| | 3A (CD16A). | DSGEYRCQTNLSTLSDPVQLEVHIGWLLLQAPRWVFKEEDPI |
| | | HLRCHSWKNTALHKVTYLQNGKGRKYFHHNSDFYIPKATL |
| | | KDSGSYFCRGLVGSKNVSSETVNITITQGLAVSTISSFFPPGY |
| | | QVSFCLVMVLLFAVDTGLYFSVKTNIRSSTRDWKDHKFKW |
| | | RKDPQDK |
| 84 | Human Fc- | MWQLLLPTALLLLVSAGMRTEDLPKAVVFLEPQWYRVLEK |
| | gamma-receptor | DSVTLKCQGAYSPEDNSTQWFHNESLISSQASSYFIDAATVD |
| | 3A (CD16A) | DSGEYRCQTNLSTLSDPVQLEVHIGWLLLQAPRWVFKEEDPI |
| | (V176F | HLRCHSWKNTALHKVTYLQNGKGRKYFHHNSDFYIPKATL |
| | polymorphic | KDSGSYFCRGLFGSKNVSSETVNITITQGLAVSTISSFFPPGY |
| | variant) | QVSFCLVMVLLFAVDTGLYFSVKTNIRSSTRDWKDHKFKW |
| | | RKDPQDK |
| 85 | NKp46-IC_F25 | EIVLTQSPATLSLSPGERATLSCRASQSVRSYLAWYQQKPGQ |
| | Fragment 1 | APRLLFSDASNRATGIPARFSGSGSGTDFTLTISSLEPEDFAV |
| | | YYCQQYRYSPRTFGQGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGECDKTHTCPPCPAPELLGGPSVFLFPPKPKDTLMISRTPE |
| | | VTCVVVDVSHEDPEVKFNWYVDGVEVHNAKTKPREEQYN |
| | | STYRVVSVLTVLHQDWLNGKEYKCKVSNKALPAPIEKTISK |
| | | AKGQPREPQVYTLPPSREEMTKNQVSLTCLVKGFYPSDIAVE |
| | | WESNGQPENNYKTTPPVLDSDGSFFLYSKLTVDKSRWQQG |
| | | NVFSCSVMHEALHNHYTQKSLSLSPGK |
| 86 | NKp46-IC_F25 | EVQLVESGGGLVQPGRSLRLSCAASGFTFDNYAMHWVRQA |
| | Fragment 2 | PGKGLEWVSGISRSSGDIDYADSVKGRFTISRDNAKNSLYLQ |
| | | MNSLRAEDTALYYCARGGVGSFDTWGQGTMVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHK |
| | | PSNTKVDKRVEPKSCDKTHTCPPCPAPELLGGPSVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPIEKTISKAKGQPREPQVYTLPPSREEMTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGSTGSQ |
| | | VQLVQSGAEVKKPGSSVKVSCKASGYTFSDYVINWVRQAP |
| | | GQGLEWMGEIYPGSGTNYYNEKFKAKATITADKSTSTAYM |
| | | ELSSLRSEDTAVYYCARRGRYGLYAMDYWGQGTTVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHS |
| 87 | NKp46-IC_F25 | DIQMTQSPSSLSASVGDRVTITCRASQDISNYLNWYQQKPGK |
| | Fragment 3 | APKLLIYYTSRLHSGVPSRFSGSGSGTDFTFTISSLQPEDIATY |
| | | FCQQGNTRPWTFGGGTKVEIKRTVAAPSVFIFPPSDEQLKSG |
| | | TASVVCLLNNFYPREAKVQWKVDNALQSGNSQESVTEQDS |
| | | KDSTYSLSSTLTLSKADYEKHKVYACEVTHQGLSSPVTKSF |
| | | NRGEC |
| 88 | moNKp46- | DIVMTQSPDSLAVSLGERATINCESSQSLLSSGNQKNYLTWY |
| | huCD123_F25 | QQKPGQPPKPLIYWASTRESGVPDRFSGSGSGTDFTLTISSLQ |
| | Fragment 1 | AEDVAVYYCQNDYSYPYTFGQGTKLEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGECDKTHTCPPCPAPELLGGPSVFLFPPKPKDTL |
| | | MISRTPEVTCVVVDVSHEDPEVKFNWYVDGVEVHNAKTKP |
| | | REEQYNSTYRVVSVLTVLHQDWLNGKEYKCKVSNKALPAP |
| | | IEKTISKAKGQPREPQVYTLPPSREEMTKNQVSLTCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| 89 | moNKp46- | EVQLVQSGAEVKKPGESLKISCKGSGYSFTDYYMKWARQM |
| | huCD123_F25 | PGKGLEWMGDIIPSSGATFYNQKFKGQVTISADKSISTTYLQ |
| | Fragment 2 | WSSLKASDTAMYYCARSHLLRASWFAYWGQGTMVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGST |
| | | GSEVQLVESGGGLVKPGGSLKLSCAASGFTFSDYGMHWVR |
| | | QAPEKGLEWVAYISSGSSTIYYADTVKGRFTISRDNAKNTLF |
| | | LQMTSLRSEDTAMYYCARGTTIFNYFEYWGQGTSVTVSSAS |
| | | TKGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKRVEPKSCDKTHS |
| 90 | moNKp46- | DIVMSQSPSSLAVSVGEKVTMSCKSSQSLLYSSNQKNYLAW |
| | huCD123_F25 | YQQKPGQSPKLLIYWASTRESGVPDRFTGSGSGTDFTLTISS |
| | Fragment 3 | VKAEDLAVYYCQQYYEIPPTFGAGTKLELKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| 91 | NKp46- | DIVMTQSPDSLAVSLGERATINCESSQSLLSSGNQKNYLTWY |
| | CD123_F5 | QQKPGQPPKPLIYWASTRESGVPDRFSGSGSGTDFTLTISSLQ |
| | Fragment 1 | AEDVAVYYCQNDYSYPYTFGQGTKLEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGECDKTHTCPPCPAPELLGGPSVFLFPPKPKDTL |
| | | MISRTPEVTCVVVDVSHEDPEVKFNWYVDGVEVHNAKTKP |
| | | REEQYNSTYRVVSVLTVLHQDWLNGKEYKCKVSNKALPAP |
| | | IEKTISKAKGQPREPQVYTLPPSREEMTKNQVSLTCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| 92 | NKp46- | EVQLVQSGAEVKKPGESLKISCKGSGYSFTDYYMKWARQM |
| | CD123_F5 | PGKGLEWMGDIIPSSGATFYNQKFKGQVTISADKSISTTYLQ |
| | Fragment 2 | WSSLKASDTAMYYCARSHLLRASWFAYWGQGTMVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGST |
| | | GSQVQLVQSGAEVKKPGSSVKVSCKASGYTFSDYVINWVR |
| | | QAPGQGLEWMGEIYPGSGTNYYNEKFKAKATITADKSTSTA |
| | | YMELSSLRSEDTAVYYCARRGRYGLYAMDYWGQGTTVTV |
| | | SSRTVAAPSVFIFPPSDEQLKSGTASVVCLLNNFYPREAKVQ |
| | | WKVDNALQSGNSQESVTEQDSKDSTYSLSSTLTLSKADYEK |
| | | HKVYACEVTHQGLSSPVTKSFNRGEC |
| 93 | NKp46- | DIQMTQSPSSLSASVGDRVTITCRASQDISNYLNWYQQKPGK |
| | CD123_F5 | APKLLIYYTSRLHSGVPSRFSGSGSGTDFTFTISSLQPEDIATY |
| | Fragment 3 | FCQQGNTRPWTFGGGTKVEIKASTKGPSVFPLAPSSKSTSGG |
| | | TAALGCLVKDYFPEPVTVSWNSGALTSGVHTFPAVLQSSGL |
| | | YSLSSVVTVPSSSLGTQTYICNVNHKPSNTKVDKRVEPKSCD |
| | | KTHS |
| 94 | IC-CD123_F5 | DIVMTQSPDSLAVSLGERATINCESSQSLLSSGNQKNYLTWY |
| | Fragment 1 | QQKPGQPPKPLIYWASTRESGVPDRFSGSGSGTDFTLTISSLQ |
| | | AEDVAVYYCQNDYSYPYTFGQGTKLEIKRTVAAPSVFIFPPS |
| | | DEQLKSGTASVVCLLNNFYPREAKVQWKVDNALQSGNSQE |
| | | SVTEQDSKDSTYSLSSTLTLSKADYEKHKVYACEVTHQGLS |
| | | SPVTKSFNRGECDKTHTCPPCPAPELLGGPSVFLFPPKPKDTL |
| | | MISRTPEVTCVVVDVSHEDPEVKFNWYVDGVEVHNAKTKP |
| | | REEQYNSTYRVVSVLTVLHQDWLNGKEYKCKVSNKALPAP |
| | | IEKTISKAKGQPREPQVYTLPPSREEMTKNQVSLTCLVKGFY |
| | | PSDIAVEWESNGQPENNYKTTPPVLDSDGSFFLYSKLTVDKS |
| | | RWQQGNVFSCSVMHEALHNHYTQKSLSLSPGK |
| 95 | IC-CD123_F5 | EVQLVQSGAEVKKPGESLKISCKGSGYSFTDYYMKWARQM |
| | Fragment 2 | PGKGLEWMGDIIPSSGATFYNQKFKGQVTISADKSISTTYLQ |
| | | WSSLKASDTAMYYCARSHLLRASWFAYWGQGTMVTVSSA |
| | | STKGPSVFPLAPSSKSTSGGTAALGCLVKDYFPEPVTVSWNS |
| | | GALTSGVHTFPAVLQSSGLYSLSSVVTVPSSSLGTQTYICNV |
| | | NHKPSNTKVDKRVEPKSCDKTHTCPPCPAPELLGGPSVFLFP |
| | | PKPKDTLMISRTPEVTCVVVDVSHEDPEVKFNWYVDGVEV |
| | | HNAKTKPREEQYNSTYRVVSVLTVLHQDWLNGKEYKCKVS |
| | | NKALPAPIEKTISKAKGQPREPQVYTLPPSREEMTKNQVSLT |
| | | CLVKGFYPSDIAVEWESNGQPENNYKTTPPVLDSDGSFFLYS |
| | | KLTVDKSRWQQGNVFSCSVMHEALHNHYTQKSLSLSPGST |
| | | GSEVQLVQSGAEVKKSGESLKISCKGSGYSFTSYWIGWVRQ |
| | | MPGKGLEWMGIFYPGDSSTRYSPSFQGQVTISADKSVNTAY |
| | | LQWSSLKASDTAMYYCARRRNWGNAFDIWGQGTMVTVSS |
| | | RTVAAPSVFIFPPSDEQLKSGTASVVCLLNNFYPREAKVQW |
| | | KVDNALQSGNSQESVTEQDSKDSTYSLSSTLTLSKADYEKH |
| | | KVYACEVTHQGLSSPVTKSFNRGEC |
| 96 | IC-CD123_F5 | EIVLTQSPGTLSLSPGERATLSCRASQSVSSSYLAWYQQKPG |
| | Fragment 3 | QAPRLLIYGASSRATGIPDRFSGSGSGTDFTLTISRLEPEDFAV |
| | | YYCQQYGSSTWTFGQGTKVEIKASTKGPSVFPLAPSSKSTSG |
| | | GTAALGCLVKDYFPEPVTVSWNSGALTSGVHTFPAVLQSSG |
| | | LYSLSSVVTVPSSSLGTQTYICNVNHKPSNTKVDKRVEPKSC |
| | | DKTHS |
| 97 | IC-hIGg1-ADCC- | EIVLTQSPATLSLSPGERATLSCRASQSVRSYLAWYQQKPGQ |
| | enh | APRLLFSDASNRATGIPARFSGSGSGTDFTLTISSLEPEDFAV |
| | Light Chain | YYCQQYRYSPRTFGQGTKVEIKRTVAAPSVFIFPPSDEQLKS |
| | | GTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 98 | IC-hIGg1-ADCC- | EVQLVESGGGLVQPGRSLRLSCAASGFTFDNYAMHWVRQA |
| | enh | PGKGLEWVSGISRSSGDIDYADSVKGRFTISRDNAKNSLYLQ |
| | Heavy Chain | MNSLRAEDTALYYCARGGVGSFDTWGQGTMVTVSSASTK |
| | | GPSVFPLAPSSKSTSGGTAALGCLVKDYFPEPVTVSWNSGAL |
| | | TSGVHTFPAVLQSSGLYSLSSVVTVPSSSLGTQTYICNVNHK |
| | | PSNTKVDKRVEPKSCDKTHTCPPCPAPELLGGPDVFLFPPKP |
| | | KDTLMISRTPEVTCVVVDVSHEDPEVKFNWYVDGVEVHNA |
| | | KTKPREEQYNSTYRVVSVLTVLHQDWLNGKEYKCKVSNKA |
| | | LPAPEEKTISKAKGQPREPQVYTLPPSREEMTKNQVSLTCLV |
| | | KGFYPSDIAVEWESNGQPENNYKTTPPVLDSDGSFFLYSKLT |
| | | VDKSRWQQGNVFSCSVMHEALHNHYTQKSLSLSPGK |

### EXAMPLES

The present invention is further illustrated by the following examples, which should not be construed as further limiting.

### Example 1. In Vitro Binding to Human CD123 and NKp46 and Tumor Killing of CD123 NKCE

CD123 NKCE exhibits high affinity for human CD123 and NKp46 targets bound through its variable regions, with subnanomolar affinity for CD123, and nanomolar affinity for NKp46. CD123 NKCE also binds to human CD16a (FcγRIIIa; as well as other Fcγ receptors) through its wild-type competent Fc domain, as shown in Table 2.

TABLE 2
CD123 NKCE affinity for human CD123, NKp46, and CD16a targets
K_{D} (nM) ± SD

| CD123 | NKp46 | CD16a (V/F) |
|---|---|---|
| 0.40 ± 0.04 | 16.3 ± 2.9 | 462 ± 12/2606 ± 91 |

Abbreviations:
K_{D}: Equilibrium dissociation constant;
SD: standard deviation;
V/F: valine/phenylalanine

CD123 NKCE binding to its targets was confirmed at the cellular level by flow cytometry, indicating binding to cell surface CD123 on the human MOLM-13 AML cell line and binding to cell surface NKp46 and CD16a on human NK cells purified from healthy donors. Co-engagement of NK cells by the CD123 NKCE through binding to NKp46 and CD16a and to CD123 on CD123-positive tumor cells leads to tumor cell killing. The CD123 NKCE leads to potent cytotoxic activity against MOLM-13 AML cells (about 20,000 CD123 receptors/cell). As expected, the CD123 NKCE cytotoxic activity varies as a function of the effector to target cell (E:T) ratio. The CD123 NKCE also leads to specific cytotoxic activity against human AML cell lines expressing lower CD123 antigen densities, ranging from 1800 to 3000 antigens per cell, using KG-1a (about 3,000 sites per cell and NB-4 (about 1800 sites per cell) (Table 3).

TABLE 3
Cytotoxic activity by fresh NK cells against AML cell lines exhibiting
different CD123 receptor densities.

| | | Geometric mean of | |
|---|---|---|---|
| | CD123 Receptor | EC₅₀ rel [CI at 95% level] | |
| AML Cell Line | Density (sites/cell) | pg/mL | pM |
| MOLM-13 | 20,000 | 637 | 4.2 |
| KG-1a | 3000 | 4526 | 30.2 |
| NB-4 | 1800 | 435 | 2.9 |

Co-engagement of NK cells and CD123-positive tumor cells by the CD123 NKCE against MOLM-13 AML cells leads to NK cell activation, degranulation, and expression of effector molecules. The CD123 NKCE mediates significant NK cell activation in the presence of MOLM-13 target cells inducing the expression of CD69, CD107, TNFα, IFNγ, and MIP1β in a concentration-dependent manner. NK cell activation was not observed in the absence of target cells, thus validating the immunological synapse-driven mechanism of activity.

CD123 NKCE is also active ex vivo against primary blasts from AML patients leading to (i) primary NK-cell activation (CD107 degranulation marker) against autologous AML blast cells, in a concentration-dependent manner using primary blood samples from AML patients at diagnosis and (ii) potent cytotoxic activity against AML blasts using healthy donor NK-cells as effectors with a relative half maximal effective concentration (EC₅₀ rel) of 3.5 ng/mL (23.2 pM).

### Example 2. Development of In Vitro and In Vivo PK/PD Models

### Rationale

Advances in immune-oncology are at the forefront of transformative cancer treatment, focusing on the specific recognition and elimination of tumor cells by the host immune system. Several targeted immunotherapies, including bispecific T cell engagers (TCEs) and natural killer (NK) cell engager (NKCE) molecules are currently under clinical evaluation for patients with acute myeloid leukemia (AML) targeting antigens frequently expressed on AML blasts, such as CD123. NK cell-based therapies represent a promising therapeutic approach for AML as a potential alternative to T-cell therapies without the frequent adverse events, such as cytokine release syndrome (CRS) or neurotoxicity. Specifically NK cell engagers are engineered, multifunctional, antibody-like molecules that co-engage tumor cells and NK cells by binding to a tumor-specific antigen and to an NK activating receptor to trigger NK cell activation and tumor cell destruction.

A trifunctional NKCE targeting the CD123 tumor antigen has recently been described. The CD123-targeting NKCE also co-engages NKp46 and CD16a (FcγRIIIa), two main activating receptors on NK cells, and has demonstrated potent antitumor activity against primary AML blasts. In preclinical studies, it has been shown to promote strong NK cell activation and induction of cytokine secretion only in the presence of AML blasts. The mechanism of action (MoA) of NKCEs is comparable to that to TCE molecules, wherein the NKCE brings the tumor cell and NK cells into closer proximity to trigger tumor cell killing by immune cells. Like TCE molecules, an NKCE drives immunological synapse formation, resulting in a bell-shaped concentration-response curve, where the width of the curve reflects the efficacy window and depends on factors governing the synapse formation (i.e., effector to tumor cell [E:T] ratio, target receptor expression, and binding affinity).

Several in vitro and in vivo mechanistic PK/PD models have been developed for either multispecific TCE or chimeric antigen receptor T-cell (CAR-T) therapies, however no model-based translational PK/PD approach has been established yet for NKCE molecules. Thus, there is a need for the development of a translational AML mechanistic synapse-driven PK/PD model for a trifunctional CD123-NKCE molecule using in vitro cytotoxicity and in vivo nonhuman primate (NHP) data.

### Methods

### Construction of Preclinical Datasets for Model Development

### In Vitro Cytotoxicity Experiments

Cytotoxicity of resting NK cells from two donors against MOLM-13 cells stained with ⁵¹Cr was evaluated at varying E:T ratios (10:1, 5:1, 1:1, and 1:5) for three incubation time periods (1, 4, and 20 hours) using eight drug concentrations (1.5E-3, 1.5E-2, 1.5E-1, 1.5, 1.5E1, 1.5E2, 1.5E3, and 1.5E4 ng/mL).

### In Vivo NHP Studies

Data from preclinical NHP PK/PD studies in monkeys after single- and repeat-dose administration (dose range: 0.5 to 3000 μg/kg via intravenous (IV) administration, and 3.6 to 1000 μg/kg via subcutaneous administration) and an in vitro PK/PD model were used to develop the in vivo NHP PK/PD model.

### In Vitro PK/PD Model for Synapse-Related Parameters for Translation

A mechanistic in vitro PK/PD model was developed as described herein. This model estimated synapse-related PD parameters for cytotoxicity (i.e., synapse potency [EC₅₀], maximal CD123+ cell killing rate constant of the synapse [kₘₐₓ], and hill coefficient [γ], with the assumptions of stable (no change over time) NK cell dynamics and no internalization assumed in the in vitro system. Parameters related to CD123-NKCE concentrations (B₀), cell numbers (EF₀ and TC₀), NKp46 and CD123 receptor densities (RTdT and RTdE), and binding affinities (KD1, KD2, k_{off1}, and k_{off2}) were fixed with values obtained either from internal company sources or the literature.

### In Vivo NHP PK/PD Model

The in vitro PK/PD model structure was expanded for the in vivo NHP PK/PD modeling. In this in vivo model, except for the values that were fixed for synapse-related PD parameters, the following additional parameters were included: plasma PK (bioavailability [F], absorption rate constant [Ka], clearance [CL], intercompartmental clearance between the central and peripheral compartments [Q], volume of distribution in the central compartment [V1], volume of distribution in the peripheral compartment [V2]), internalization of the binary complex of trifunctional antibody-CD123 (kᵢₙₜ), synapse-driven NKp46+NK cell redistribution (Slp_{NK}), anti-drug antibody (ADA) effect on PK (rate constant for drug elimination due to ADAs [K_{ADA}] and typical time to ADA emergence [T_{ADA}]), and tolerance feedback to CD123+ cell regrowth (kₜₒₗ and δ). In addition, it was assumed that the trifunctional antibody bound spontaneously to CD123 expressed in compartments other than blood, leading to the formation of a binary complex of the trifunctional antibody and CD123 that underwent internalization. The turnover of this additional CD123 expression was described by including kᵢₙTP and kₒᵤₜTP parameters. Of the 53 NHPs evaluated in the NHP studies, PK and PD data from 23 NHPs were included in the NHP PK/PD modeling. The remaining 30 NHPs receiving 100 and 3000 μg/kg, IV, twice a week (b.i.w.), and 3000 μg/kg, SC, b.i.w. of CD123 NKCE, PK data were included for model development while PD data were used to perform external validation. Observed baseline cell parameters (EF₀ and TC₀) were mapped as regressor variables in the model. The data of censored PK and total CD123+ cells that were below the limit of quantification (BLQ) were handled using the M3 method (Bergstrand, M. and M. O. Karlsson, Handling data below the limit of quantification in mixed effect models. Aaps j, 2009. 11(2): p. 371-80).

### AML Translational PK/PD Model

The in vivo NHP PK/PD model was translated to AML patients by assuming a model structure like that for NHPs with few exceptions, which were as follows: replacing tolerance feedback on tumor growth with a maximum capacity of CD123+ cells (TCₘₐₓ) and applying PK allometric scaling with exponents 0.81, 0.57, 1.04, and 1.07 for C_{L}, Q, V1, and V2, respectively. The system-specific and binding affinity-related parameters were adapted from the values known for either patients or healthy subjects (e.g., EF₀, TC₀, TCₘₐₓ, NK cell turnover rate constant [kₒᵤₜ], CD123+ cell proliferation constant [k_{g}], RTdE, RTdT, KD1, and KD2). In contrast, biological-specific parameters that were unknown in patients (e.g., kᵢₙTP, a, and 3) or drug-specific parameters without a clear scaling approach (e.g., kit, Slp_{NK}, kₘₐₓ, EC₅₀, and γ) were assumed to be the same as those for NHPs. Of note, the ADA effect on CD123-NKCE clearance was not included in the AML PK/PD model, as ADA occurrence is unlikely in humans due to the use of a humanized CD123-NKCE construct.

### Starting Dose Prediction

Simulations were performed to predict a starting IV dose of CD123-NKCE that would meet two criteria: i) the maximum concentration at the end of a single-dose infusion (Cₑₒᵢ) would be lower than the minimum concentration that was found to induce mild cytokine release in a whole-blood cytokine release assay (1.7 nmol/L); and ii) a decrease of the CD123+ cell count by >95% at the end of the first treatment cycle (i.e., 28 days) following b.i.w. dosing. This dose prediction approach assumed that the intensity of the cytokine release syndrome (a rapid adverse reaction) is dependent on the maximum concentration (Cₘₐₓ) achieved after a single dose of a biologic.

### Sensitivity Analyses

To evaluate the uncertainties of the model related to efficacy at the projected starting dose, a GSA with extended Fourier amplitude sensitivity test (eFAST) (A. Saltelli, S. Tarantola & K. P.-S. Chan (1999) A Quantitative Model-Independent Method for Global Sensitivity Analysis of Model Output, Technometrics, 41:1, 39-56, DOI: 10.1080/00401706.1999.10485594) and an LSA were performed. This allowed the estimation of uncertainty in the model's predictions for PK and percent change from baseline (% CFB) in CD123+ cell count at the selected FIH starting dose.

### Results

### Characterization of In Vitro Data

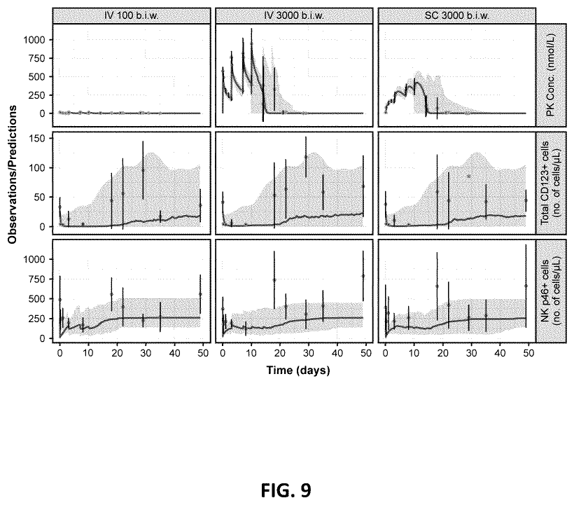

The observed in vitro cytotoxicity data against MOLM-13 cells after incubation with CD123-NKCE at different concentrations, incubation time intervals, and varying the E:T ratios were fitted by an in vitro PK/PD model framework (FIG. 4A). A model was used to estimate the PD parameters by fixing the k_{g} and receptor density of CD123 (RTdT) with known values (Table 2.)

TABLE 4
Summary of parameters that were fixed or estimated to develop the preclinical
(in vitro and in vivo) and translated clinical PK/PD models for CD123 NKCE.

| | | In vitro | | | | | |
|---|---|---|---|---|---|---|---|
| | | PK/PD model | | | In vivo NHP | | AML |
| | | (MOLM-13) | | | PK/PD model | | PK/PD |
| | | Typical | | IIV | Typical | IIV | model |
| | | Estimate | | Estimate^{a} | Estimate | Estimate^{a} | Typical |
| Parameter | Description | (RSE %) | | (RSE %) | (RSE %) | (RSE %) | Estimate |
| B₀ | In vitro CD123-NKCE | 1.02E−5-102^{b} | | | | | |
| | concentration (nmol/L) | | | | | | |
| EF₀ | In vitro/baseline NK | 4-200^{b} | | | Median = 247 | | 54^{c} |
| | cell density (cells/μL) | | | | [144-1110]^{b} | | |
| TC₀ | In vitro/baseline | 20^{b} | | | Median = 28 | | 1668^{c} |
| | CD123+ cell density | | | | [1.59-103]^{b} | | |
| | (cells/μL) | | | | | | |
| RTdE | NKp46 receptor | 4600^{c} | | | 4600^{c} | | 4600^{c} |
| | density (receptors/cell) | | | | | | |
| RTdT | CD123 receptor | 4360 Fixed^{c} | | | 25,000 Fixed | | 7000^{d} |
| | density (receptors/cell) | | | | | | |
| kₘₐₓ | Maximal cytotoxicity | 0.332 | (8.42)^{e} | | 0.332^{f} | | 0.332^{f} |
| | rate constant (1/h) | | | | | | |
| EC₅₀ | Synapse potency | 1.46E−9 | (21.7)^{e} | | 1.46E−09^{f} | | 1.46E−09^{f} |
| | (nmol/L) | | | | | | |
| γ | Hill coefficient (—) | 1.04 | (0.153)^{e} | 0.998 (14.2)^{d} | 1.04^{f} | | 1.04^{f} |

| k_{g} | CD123+ cell growth | 0.0139^{g} | 0.31 | (50%)^{e} | 0.83 | (26.8%)^{e} | 0.0288^{h} |
|---|---|---|---|---|---|---|---|
| | rate (1/h) | | | | | | |
| kₒᵤₜ | NKp46+ NK cell | | 0.2ⁱ | | | | 0.002^{j} |
| | turnover rate constant | | | | | | |
| | (1/h) | | | | | | |
| KD1 | NKp46 binding affinity | 16.6^{k} | 29^{c} | | | | 16.6^{c} |
| | (nmol/L) | | | | | | |
| KD2 | CD123 binding affinity | 0.404^{k} | 1.23^{c} | | | | 0.404^{c} |
| | (nmol/L) | | | | | | |
| CL | Clearance (mL/h/kg) | | 0.32 | (7.32)^{e} | 0.20 | (31.2)^{e} | 0.18^{l} |
| Q | Intercompartmental | | | | | | |
| | distribution rate | | 1.35 | (4.24)^{e} | | | 0.37^{l} |
| | constant (mL/h/kg) | | | | | | |
| V1 | Volume of central | | 0.040 | (4.23)^{e} | 0.130 | (31.3)^{e} | 0.042^{l} |
| | compartment (L/kg) | | | | | | |
| V2 | Volume of peripheral | | 0.020 | (14.3)^{e} | 0.26 | (57.8)^{e} | 0.031^{l} |
| | compartment (L/kg) | | | | | | |
| kᵢₙₜ | Internalization rate of | | 0.15 | (1.76%)^{e} | | | 0.15^{m} |
| | trifunctional antibody- | | | | | | |
| | CD123 binary complex | | | | | | |
| | (1/h) | | | | | | |
| k_{off1} | Dissociation rate | 0.001 Fixedⁿ | 0.001ⁿ | | | | 0.001ⁿ |
| | constant for binding of | | | | | | |
| | CD123-NKCE to | | | | | | |
| | NKp46 (1/s) | | | | | | |
| k_{off2} | Dissociation rate | 0.001 Fixedⁿ | 0.001ⁿ | | | | 0.001ⁿ |
| | constant for binding of | | | | | | |
| | CD123-NKCE to | | | | | | |
| | CD123 (1/s) | | | | | | |
| kₐ | Absorption rate | | 0.04 | (9.12)^{e} | | | |
| | constant (1/h) | | | | | | |
| F | Bioavailability (%) | | 74.0 | (6.87)^{e} | 0.28 | (55.2)^{e, o} | 3252^{p} |
| TCₘₐₓ | Maximum capacity of | | | | | | |
| | CD123+ cells | | | | | | |
| | (cells/μL) | | | | | | |
| Slp_{NK} | Slope of synapse- | | 5.72 | (0.951%)^{e} | | | 5.72^{m} |
| | driven NKp46+ NK | | | | | | |
| | cell redistribution | | | | | | |
| | (1/mol/L) | | | | | | |
| kᵢₙTP | Production rate | | 0.21 | (15.4%)^{e} | 0.57 | (22.6%)^{e} | 0.21^{m} |
| | constant for extra | | | | | | |
| | CD123+ expression | | | | | | |
| | (nmol/L/h) | | | | | | |
| T_{ADA} | Onset time of emergent | | 333 | (4.11)^{e} | 0.20 | (15.13)^{e} | |
| | ADAs (h) | | | | | | |
| K_{ADA} | CD123-NKCE | | 1.94 | (84.9)^{e} | 4.21 | (17.7)^{e} | |
| | elimination rate | | | | | | |
| | constant due to | | | | | | |
| | emergent ADAs (1/h) | | | | | | |
| kₜₒₗ | Tolerance feedback | | 0.0032 | (55.9%)^{e} | 1.63 | (43.6%)^{e} | |
| | rate constant of | | | | | | |
| | CD123+ cell regrowth | | | | | | |
| | (1/h) | | | | | | |
| δ | Tolerance feedback | | 0.18 | (32.4%)^{e} | 0.53 | (35.5%)^{e} | |
| | power coefficient (—) | | | | | | |
| α | Intercept of correlation | | 19.44 | (16.3%)^{e} | 0.61 | (19.8%)^{e} | 19.44^{m} |

| | | | | |
|---|---|---|---|---|
| | of CD123 expression | | | |
| | between blood and | | | |
| | other tissues (nmol/L) | | | |
| β | Exponent of correlation | 0.21 | (0.842%)^{e} | 0.21^{m} |
| | of CD123 expression | | | |
| | between blood and | | | |
| | other tissues (—)“vs” | | | |
| | Exponent of TP0- | | | |
| | blood CD123 | | | |
| | expression correlation | | | |

Abbreviations:
ADA, Anti-drug antibody; AML, Acute myeloid leukemia; CD, Cluster of differentiation; IIV, Interindividual variability; NHP, Nonhuman primate; NK, Natural killer; PD, Pharmacodynamic; PK, Pharmacokinetic; RSE, Relative standard error.
^{a}Standard deviation of IIV random effect;
^{b}Source: Actual concentrations as regressor;
^{c}Source: Internal data;
^{d}Source: Baroni, M. L., et al., 41BB-based and CD28-based CD123-redirected T-cells ablate human normal hematopoiesis in vivo. J Immunother Cancer, 2020. 8(1).;
^{e}Source: Estimated;
^{f}Source: Estimation from the in vitro PK/PD model;
^{g}Source: Doubling time ~50 h;
^{h}Source: Based on doubling time of 24 h;
ⁱSource: Calibrated;
^{j}Source: Stringaris, K., Orphan NKs! The mystery of the self-renewing NK cells. Blood, 2017. 129(14): p. 1890-1891.;
^{k}Source: Internal measurement for humans;
^{l}Source: Scaled from NHP data;
^{m}Source: Estimation from NHP data;
ⁿSource: Jiang, X., et al., Development of a Target cell-Biologics-Effector cell (TBE) complex-based cell killing model to characterize target cell depletion by T cell redirecting bispecific agents. MAbs, 2018. 10(6): p. 876-889.;
^{o}Logit model of IIV random effect;
^{p}Assumed 95% higher than that at baseline.

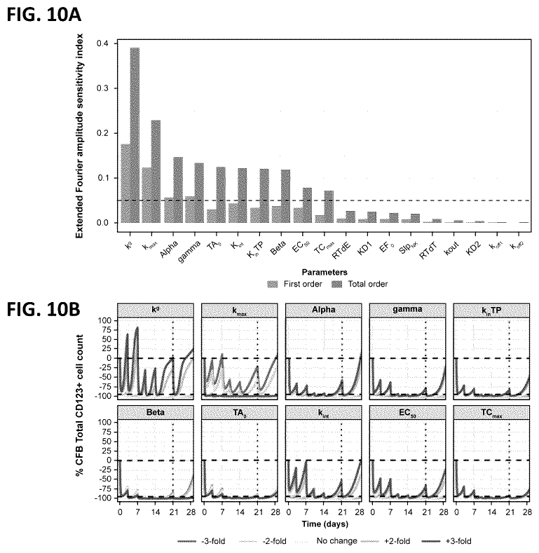

The model fits showed that the data were well fitted at different incubation time points, except for slight overprediction of the data from the E:T ratios of 1:1 and 1:5 at 20 h of incubation (FIG. 5). This can be explained by the spontaneous release of ⁵¹Cr from radiolabeled MOLM-13 cells of these affected data, in which the original values were negative and were required to be normalized using controlled measurements before they can be used for modeling.

### Characterization of In Vivo PK/PD Data of CD123 NKCE in NHPs

Given the substantial target-mediated drug disposition (TMDD) and ADAs observed in NHPs, the in vivo NHP PK/PD model was developed in a sequential manner, beginning with fitting the data from 3000 μg/kg IV and SC CD123-NKCE dosing at timepoints <1 week that exhibited linear PK without the impact of ADA, for ensuring that the linear PK parameters (CL, Q, V1, V2, F, and ka) were estimated with high precision (Table 1).

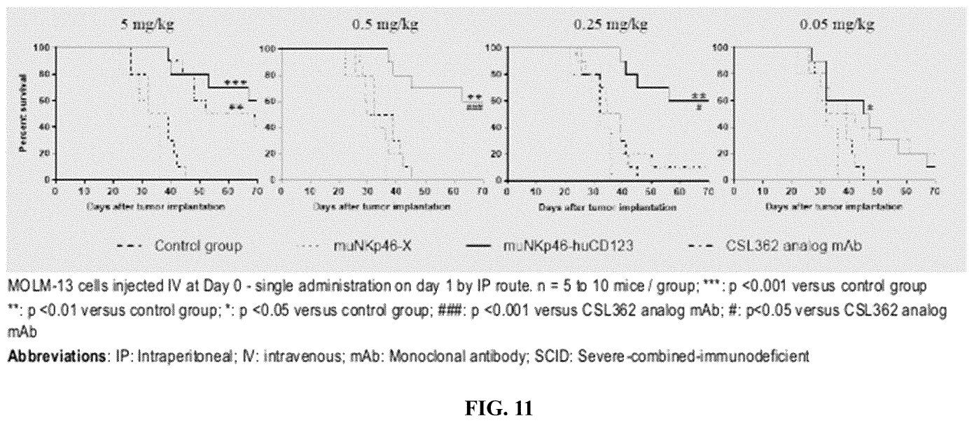

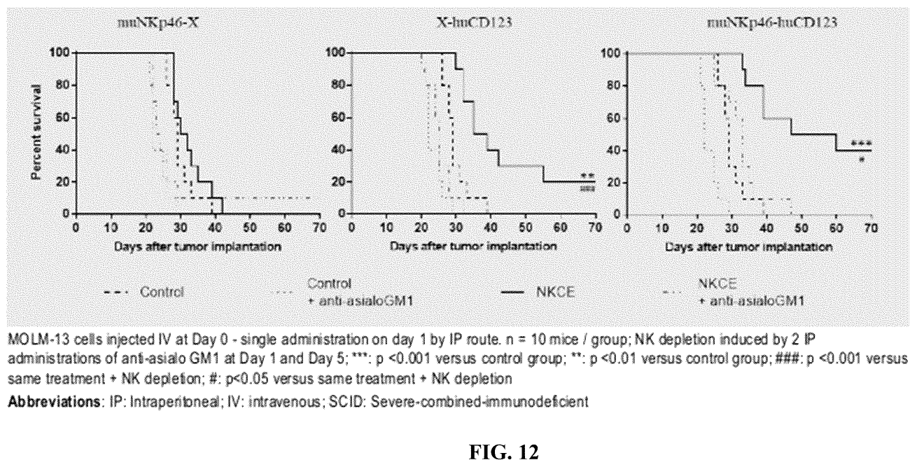

The remaining timepoints (i.e., ≥1 week) from the 3000 μg/kg IV and SC dosing data were included in the model to characterize the ADA effects. The model estimated T_{ADA} at ˜2 weeks (Table 1), which is consistent with the time observed for ADA onset (FIG. 6). K_{ADA} was estimated to be ˜400-fold higher than the nonspecific elimination rate constant, CL/(V1+V2) (1.94 l/h versus 0.005 l/h). A high interindividual variability (IIV) observed in the NHP PK at timepoints >2 weeks (FIG. 7A) can be explained by the heterogeneity of NHPs in responding to the administration of a human trifunctional antibody (Table 1).

Upon inclusion of all the PK data for modeling, the model was required to consider the binding of CD123-NKCE with additional CD123 receptors that were expressed in compartments other than blood to characterize TMDD. Without this assumption, it was observed that the model consistently overpredicted PK profiles at doses exhibiting TMDD (data not shown). Therefore, an additional turnover model was included to characterize this hypothetical additional CD123 expression. The observed baseline CD123+ cell count in the blood was directly correlated with the baseline extra CD123 expressed elsewhere depicted by a scaling function parameterized as α and β.

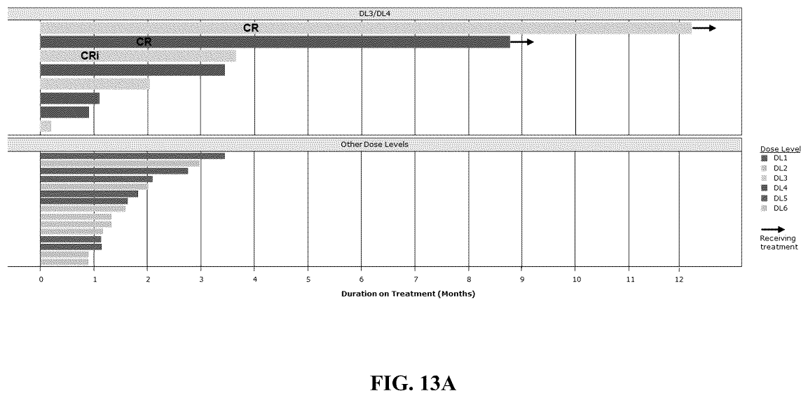

The individual CD123+ cell count profiles showed that an early rebound of CD123+ cell depletion was generally associated with an early development of ADAs. Of all the animals demonstrating rebound at ˜2 weeks, one animal had minimal ADAs and showed protracted CD123+ cell depletion compared with other NHPs which exhibited a high CD123-NKCE clearance in the presence of ADAs (FIG. 8). Therefore, it is important to characterize individual ADA effects on PK before the model can characterize the CD123+ cell profiles adequately. CD123+ cell count data during the rebound phase were satisfactorily fitted in visual predictive checks (VPCs) (FIG. 6B).

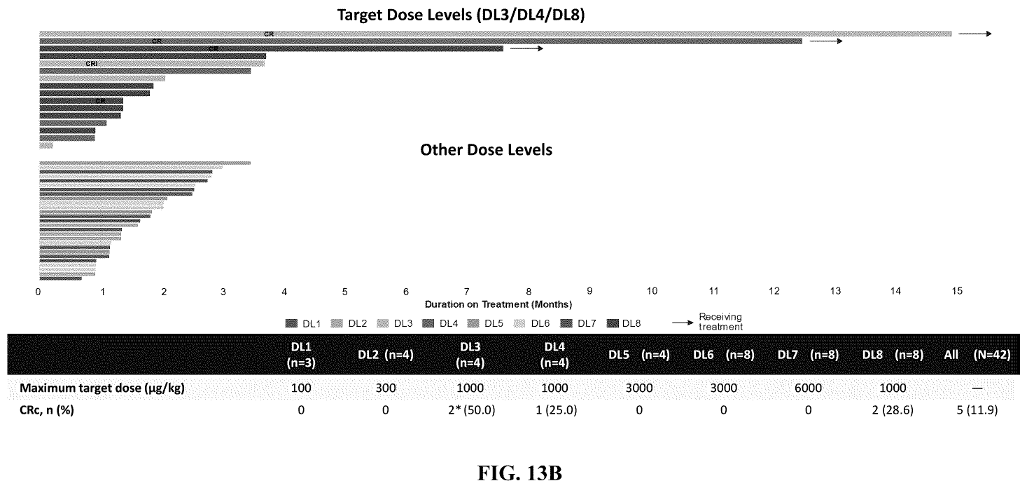

Because healthy NHPs were studied, the CD123+ cell rebound was eventually counterbalanced by a tolerance feedback mechanism, thereby returning to baseline readings. Hence, a moderator-mediated tolerance model was incorporated to act on parameter k_{g}. According to the external validation using b.i.w. data, the model predicted CD123+ cell depletion adequately but underpredicted the data during the rebound phase even after extremely high cell counts (>200 cells/μL) were excluded (FIG. 9).

The observed NKp46+NK cell decreased in the blood following CD123-NKCE administration can be explained by a synapse-driven NK cell redistribution phenomenon. The NKp46+NK cell turnover model which included a stimulated linear slope function of synapse concentration on kₒᵤₜ(FIG. 4B) showed to fit the observations adequately (FIG. 6C). According to the estimation, NKp46+NK cell redistribution increased with an increase in synapse concentration at a rate of 5.72 l/(mol/L) (Table 1).

Overall, the model was qualified (FIG. 6A-6C), and the parameters were estimated with a good level of precision (Table 1).

### Translated AML PK/PD Model

CD123-NKCE linear PK parameters C_{L}, Q, V1, and V₂ that were allometrically scaled from NHP to human were 0.18 mL/h/kg, 0.37 mL/h/kg, 0.042 L/kg, and 0.031 L/kg, respectively (Table 1).

The following inputs were applied to the translated AML model from the NHP model: 1) lower EF0 and higher TC0 counts, resulting in a much lower E:T ratio; 2) a lower value of RTdT; 3) binding affinity (KD1 and KD2) values obtained from in vitro experiments using human cells; 4) a recalculated k_{g} based on 24-h doubling time; 5) a kₒᵤₜ calculated based on 14 h of half-life; and 6) an assumption of the TCₘₐₓ of CD123+ cells in the blood being 95% higher than the baseline value.

### Uncertainties of the Safety and Efficacy Predictions at the Projected Starting Dose

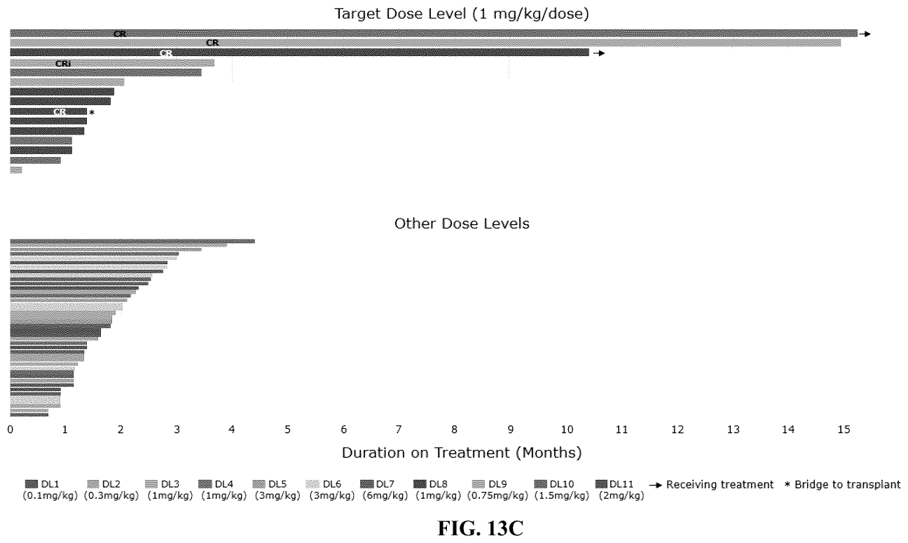

Of all the parameters examined using GSA, those governing CD123+ cell growth (k_{g}), tumor killing by synapse formation (kₘₐₓ, EC₅₀, and γ), internalization rate constant with CD123 receptors (kᵢₙₜ), additional CD123 expression (α, β, and kᵢₙTP), baseline counts of CD123+ cells (TA₀), and CD123+ cell TCₘₐₓ were shown to significantly impact the tumor cell killing predictions (the eFAST total-order indices were >0.05; FIG. 10A).

According to the LSA results, k_{g} and kₘₐₓ were the most sensitive parameters, whereas α, γ, kᵢₙTP, kᵢₙₜ, and EC₅₀ were marginally sensitive, followed by others that were less sensitive (FIG. 6B). These results were largely consistent with the ranking of total-order indices of GSA, as depicted in FIG. 6A. Notably, a >2-fold increase in k_{g} or a >2-fold decrease in kₘₐₓ would result in an unsustainable CD123+ cell depletion of >95% throughout the first treatment cycle (FIG. 10B). For α, γ, kᵢₙTP, kᵢₙₜ, and EC₅₀, increasing the parameter values would mainly affect CD123 depletion during the rebound phase occurring 21 days after the first CD123-NKCE dose (FIG. 10B).

### Simulations for Starting Dose Prediction

Simulations were performed to predict the PK and % CFB (Change From Baseline) in CD123+ cell counts following b.i.w. dosing with CD123-NKCE. A fixed dose of at least 100 μg/kg was needed to yield a >95% decrease in CD123+ cell counts in the first dosing cycle (FIG. 7A). Nevertheless, this first dose of 100 μg/kg potentially induces cytokine release, as the predicted Cₑₒᵢ exceeds the CD123-NKCE minimum concentration level observed cytokine release from an in vitro whole-blood assay (1.7 nmol/L) (FIG. 7B). To reduce the risk of any adverse events (such as infusion-related cytokine release), we simulated intra-patient dose escalation of 10 and 30 μg/kg in the first week, followed by 100 μg/kg escalations (10/30/100). With this regimen, the predicted Cₑₒᵢ after a single dose was approximately one-log value lower than the target concentration (FIG. 7B) while retaining the efficacy of the fixed 100 μg/kg dose (FIG. 7A).

Based on the mechanistic PK/PD model, the estimated single dose exposure (Cₘₐₓ, AUC0-3d) in human at 1/10th (safety factor) of NOAEL, ie, 3000 μg/kg/adm after 5-week twice weekly dosing in cynomolgus monkey, corresponds to human doses ranging from 1000 to 3000 μg/kg. Considering the favorable nonclinical safety findings, a starting dose of 10 μg/kg would provide a reasonable safety margin, and the estimated human single exposure at 10 μg/kg.

### Example 3. In Vivo Efficacy of CD123 NKCE in a Murine Model

To evaluate the in vivo efficacy of CD123 NKCE, a murine surrogate (muNKp46-huCD123) molecule was generated, allowing the NKp46 arm of the CD123 NKCE to bind with murine NKp46. To generate the murine reactive surrogate, the human reactive NKp46 arm of the CD123 NKCE was replaced with a version that binds to the murine NKp46 protein. The human CD123 reactive arm and human IgG1 Fc domain were retained. The human IgG1 competent Fc domain retains binding to all murine FcγRs and is capable of recruiting murine NK-cells to induce antibody-dependent cellular cytotoxicity (ADCC). The in vivo efficacy of the murine surrogate was evaluated in severe-combined-immunodeficient (SCID) mice engrafted with disseminated human MOLM-13 tumor cells.

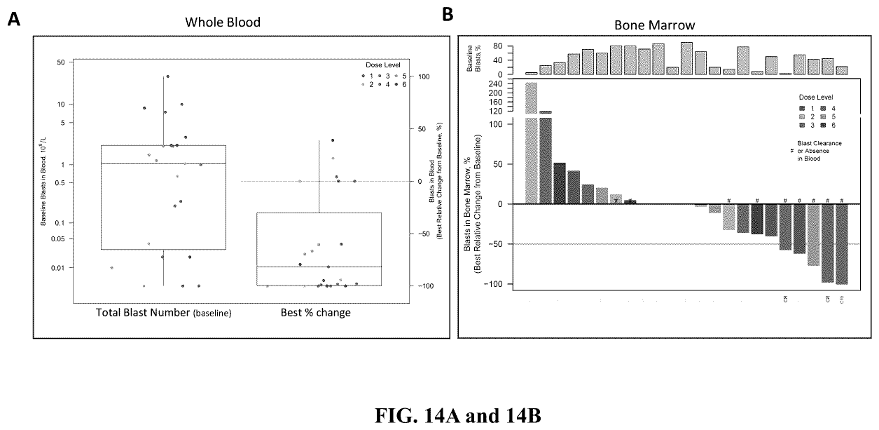

The muNKp46-huCD123 activity was compared to an analog monoclonal antibody (mAb) (an Fc-engineered anti-CD123 antibody for enhanced ADCC), able to bind to murine FcγRs and to recruit murine effector cells). Mice were intravenously inoculated with tumor cells on Day 0. Treatments were administered intraperitoneally on Day 1 post tumor implantation. Data were summarized with Kaplan-Meier curves and survival differences between groups were evaluated with a Cox model and are presented in FIG. 11.

The muNKp46-huCD123 NKCE induced statistically significant activity at doses of 5, 0.5, 0.25, and 0.05 mg/kg in human MOLM-13 disseminated model, with a percent increased lifespan (ILS) compared to control of 100% and 60% of long-term survivors for the doses of 5, 0.5, and 0.25 mg/kg and an ILS of 30% and 10% of long-term survivors for the dose of 0.05 mg/kg. The analog mAb induced a statistically significant activity at the dose of 5 mg/kg in human MOLM-13 disseminated model, with an ILS of 70% and 40% of long-term survivors. It was not active at the doses of 0.5, 0.25, and 0.05 mg/kg.

The muNKp46-huCD123 NKCE show more activity than the analog mAb at the doses of 0.5 and 0.25 mg/kg (statistically significant), demonstrating the benefit of co-engaging NK-cells with NKp46/CD16.

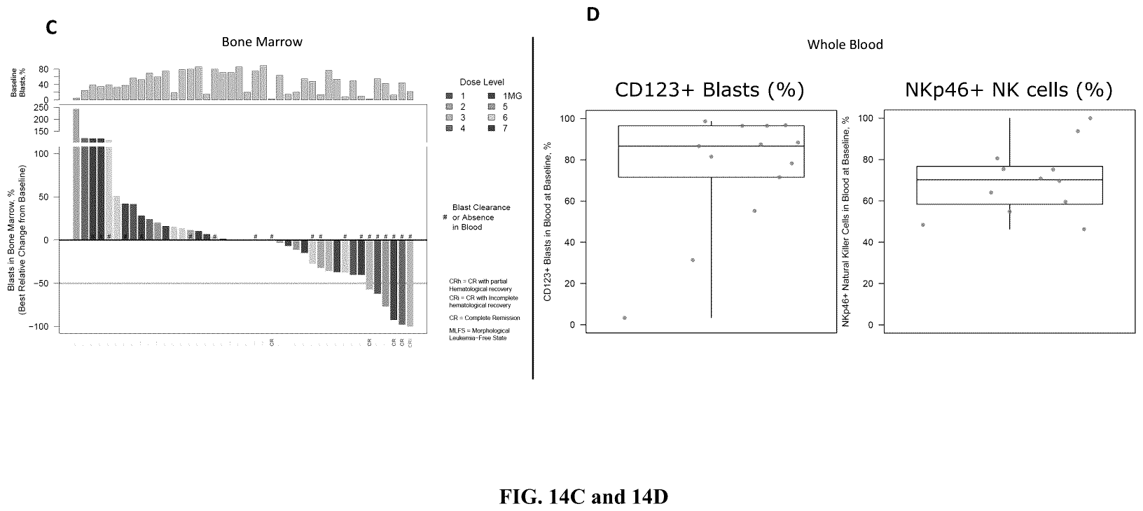

Additional experiments in the same mouse model demonstrated that the anti-tumor activity was dependent on NK-cells. Controls including an isotype control antibody binding muNKp46 and murine FcγRs but not huCD123 (NKp46-X) and a second isotype control antibody binding huCD123 and murine FcγRs but murine NKp46 (X-huCD123) were also evaluated. As shown in FIG. 12, muNKp46-huCD123 engaging NK-cells via mFcγRs and NKp46 showed more activity than X-huCD123 isotype control able to induce NK-cells only via mFcγRs. The antibody-mediated depletion of murine NK-cells led to a significant loss of anti-tumor activity of both NKCEs, confirming the NK-cell involvement as effector cells in muNKp46-huCD123.

### Example 4. Pharmacokinetics and Pharmacodynamics of CD123 NKCE in Cynomolgus Monkeys after Single Dose

### Pharmacokinetics

The objective of these studies was to determine CD123 NKCE PK parameters from plasma concentration in monkeys after a single administration, given as IV (1-hour infusion) at 0.5, 3, and 3000 μg/kg or SC administrations at 3.6 and 1000 μg/kg. Toxicokinetic (TK) parameters were calculated after a once weekly repeated administration for 4 weeks and after a twice weekly repeated administration for 5 weeks at 100 and 3000 μg/kg/adm toxicity studies and after a twice weekly repeated SC administration at 3000 μg/kg/adm. The PK parameters of CD123 NKCE obtained from plasma concentrations after a single 1-hour infusion IV and SC administration are presented in Table 5 below. After a single 1-hour IV infusion, from 0.5 μg/kg up to 3000 μg/kg, plasma concentrations at the end of infusion (Cₑₒᵢ) and exposure (AUC) increased more than expected by dose proportionality due to TMDD that seemed to be saturated at the highest dose.

TABLE 5
Pharmacokinetic parameters in cynomolgus monkeys (mean, CV).
CD123 NKCE cynomolgus monkey (serial sampling, n = 2 IV, n = 3 SC) - Plasma

| | | | | Cₑₒᵢ/ | | AUC | CL | | | |
|---|---|---|---|---|---|---|---|---|---|---|
| | | | Dose | Cₘₐₓ | tₘₐₓ | (day · ng/ | (ml/day/ | Vₛₛ | t_{1/2z} | |
| Species | Sex | Route | (μg/kg) | (ng/mL) | (h) | mL) | kg) | (mL/kg) | (day) | F % |
| Monkey | M | IV | <0.5 | 5.48 | 1 | NC | NC | NC | NC | N/A |
| | | | 0.5 | 10.6 | 1 | 1.16 | 429 | 33.3 | 0.0657 | N/A |
| | | | 3 | 55.0 | 1 | 9.12 | 342 | 126 | 0.988 | N/A |
| | | | 3000 | 91,600 | 1.2 | 501,000 | 5.99 | 49.2 | 6.05 | N/A |
| | | SC | 3.6 | 3.29 | 6 | NC | N/A | N/A | N/A | NC |
| | | | 1000 | 6250 | 48 | 64,600 | N/A | N/A | N/A | 33.3 |

Abbreviations:
Cₘₐₓ: Maximum concentration observed;
tₘₐₓ: time of maximum observed concentration,
AUC: Area under the curve;
CL: total plasma clearance;
Vss: Volume of distribution at steady state;
n = 1 due to the presence of ADA (Anti Drug Antibody) preventing accurate estimation of PK parameters

Following a single IV administration (1-hour infusion) to cynomolgus monkeys, Target Mediated-Drug Disposition (TMDD) was evidenced after CD123 NKCE infusion from the lowest

dose (0.5 μg/kg) up to 3 μg/kg, while at high doses (3000 μg/kg IV and 1000 μg/kg SC), the target appeared saturated, allowing to estimate CD123 NKCE linear clearance at 6 mL/day/kg and volume of distribution around 50 mL/kg, with a terminal half life of 6 days. (It may be noted that the PK parameters were sometimes extrapolated due to ADA response leading to possible under-estimation of t½z; using the PK/PD model built in NHP, t½z was evaluated to be 8 days.)

After single SC administration (1000 μg/kg), maximal plasma concentration of CD123 NKCE was observed between 24- and 48-hours post-administration and bioavailability (based on AUC0-7d) was estimated close to 35% at target saturated dose.

Anti-drug antibody (ADA) impact on exposure was observed at high doses following both single IV infusion (3000 μg/kg) and SC (1000 μg/kg) administrations depicted by a sharp drop in concentrations from 7 to 10 days after both IV and SC administrations. At low doses, TMDD led to concentrations below the limit of quantification on Day 7.

### Pharmacodynamics

After CD123 NKCE infusion, from 0.5 to 3000 μg/kg, a dose effect on CD123+ depletion could be evidenced, occurring rapidly from the first sampling time (1.5 hours post start of infusion), maintained up to 5 hours (i.e. 0.21 days) at 0.5 μg/kg and sustained up to 14 and 28 days at 3 and 3000 μg/kg for total cells and basophils, respectively.

After a single 1-hour IV infusion, from 0.5 μg/kg up to 3000 μg/kg, plasma concentrations at the end of infusion (Cₑₒᵢ) and exposure (AUC) increased more than expected by dose proportionality due to TMDD that appeared to be saturated at the highest dose. See Table 6.

TABLE 6
Summary of monkey pharmacokinetic parameters. Serial sampling, n = 2 IV, n = 3 SC; plasma.

| | | | | Cₑₒᵢ/ | | AUC | CL | | | |
|---|---|---|---|---|---|---|---|---|---|---|
| | | | Dose | Cₘₐₓ | Tₘₐₓ | (day · ng/ | (mL/day/ | Vₛₛ | T_{1/2z} | |
| Species | Sex | Route | (μg/kg) | (ng/mL) | (h) | mL) | kg) | (mL/Kg) | (day) | F % |
| Monkey | M | IV | <0.5 | 5.48 | 1 | NC | NC | NC | NC | NA |
| | | | 0.5 | 10.6 | 1 | 1.16 | 429 | 33.3 | 0.0657 | NA |
| | | | 3 | 55.0 | 1 | 9.12 | 342 | 126 | 0.988 | NA |
| | | | 3000 | 91600 | 1.2 | 501000 | 5.99 | 5.99 | 6.05 | NA |

| | | | | | | | | | |
|---|---|---|---|---|---|---|---|---|---|
| SC | 3.6 | 3.29 | (80) | 6 | NC | NA | NA | NC | NC |
| | 1000 | 6250 | (11) | 48 | 64600 | NA | NA | NC | 33.3 |

Abbreviations:
ADA: anti-drug antibody;
AUC: Area under the plasma concentration;
Cₑₒᵢ: Concentration observed at the end of infusion;
CL: total plasma clearance;
Cₘₐₓ: Maximum concentration observed;
CV: Coefficient of variation in brackets;
F %: Absolute bioavailability calculating using AUC_{0-7 d} (34400 day · ng/mL after SC at 1000 μg/kg and 310000 day · ng/mL after IV at 3000 μg/kg) (expressed as a percentage);
NC: not calculated;
NA: not applicable;
Vss: Volume of distribution at steady state after single dose;
tₘₐₓ: time of maximum observed concentration

After single SC administration (3.6 and 1000 μg/kg), maximal plasma concentration of CD123 NKCE was observed between 6- and 48-hours post-administration. Depending on dose, the bioavailability (F %) of CD123 NKCE was estimated to be close to ˜35% at 1000 μg/kg. After twice weekly SC administration at 3000 μg/kg/adm for 5 weeks, mean bioavailability ranged from ˜40% to 65%, respectively on Days 1, 4, and 8. It may be noted that using the PK/PD model, F was 48% and the absorption rate (ka) was 0.078 h⁻¹.

### Example 5. In Vitro Cytokine Release Assay in Whole Blood-Aqueous Phase

The potential of CD123 NKCE to induce cytokine release (CR) was evaluated in a whole

blood-aqueous phase (WB-AQ) cytokine release assay (CRA), a relevant human in vitro assay designed to inform potential risk for the occurrence of CRS in humans.

The study included a negative control (untreated whole blood) and a positive control known to mediate CR through engagement of CD16 on NK-cells and for which the whole blood CRA is most sensitive, a humanized anti-CD52 IgG, tested at 100 μg/mL. The study also included a relevant tool compound with similar mechanism of action (depletion of CD123 positive cells), a bi-specific T-cell engager (TCE) antibody (CD3/CD123), as a positive control (tested at 10 μg/mL).

The study was carried out using whole blood from a cohort of 13 healthy human donors. In this study, CD123 NKCE was evaluated using a broad range of concentrations, from 0.000025 to 100 μg/mL. The cytokine concentrations (pg/mL) of IFNγ, TNFα, IL-2, IL-13, IL-6, GM-CSF, IL-4, IL-8, MIP1β, and IL-10 were measured in plasma from each donor after overnight

treatment (˜24 hours), and following centrifugation, using a Meso Scale Discovery (MSD) platform. For each donor, the mean (with standard deviation [SD]) and median [Q1, Q3]) fold change for each cytokine was calculated relative to untreated whole blood (negative control). In
addition, the total number of CD123+ cells in each sample were determined by flow cytometry and expressed as a percent relative to the amount present in untreated whole blood, and the mean
(SD) for each treated sample was calculated.

Across all donors, a wide variability was observed with respect to fold increases in cytokine levels. This is not unexpected for whole blood CRA where all blood cell types and plasma (including native immunoglobulins and complement proteins) are present and can impact cytokine levels.

The mean fold increase across the 13 donors observed for IFNγ, TNFα, and IL-6 following treatment with a humanized anti-CD52 IgG1 was at or near the reference values provided by NIBSC in whole blood treated with this compound for 35 donors.

Following treatment with CD123 NKCE, there was no meaningful increase in GM-CSF, IL-10, IL-13, IL-4, IL-8, and MIP1β, however, IFNγ and IL-6 were observed to increase in vitro in human whole blood, consistent with engagement of NK-cells. TNFα, which is also increased following engagement of NK-cells and treatment with the humanized anti-CD52 IgG1, did not show significant increase following CD123 NKCE treatment. The observed release of IFNγ was below the level observed with the humanized anti-CD52 IgG1 (mean fold change of 42) at all concentrations of CD123 NKCE evaluated, and IL-6 was similar to levels induced by the humanized anti-CD52 IgG1 (mean fold change of 12) at and above the concentration of 0.25 μg/mL (below this concentration, minimal increases in IL-6 were observed). Furthermore, the increase in both IL-6 and IFNγ plateaued and did not increase with CD123 NKCE concentrations tested above 0.25 μg/mL (up to 100 μg/mL).

For comparison, and because of the large donor to donor variability observed with most of the cytokines evaluated, the median fold change was also calculated. Using the median fold change, IL-6 was only increased at the two highest CD123 NKCE concentrations tested (50 and 100 μg/mL).

The observed cytokine release with CD123 NKCE was much lower than that observed with the CD3-CD123 TCE tool compound, where all cytokines evaluated were increased (mean fold changes ranging from 2 to >5000 for the 13 donors).

Decreased numbers of CD123+ cells, indicative of the expected pharmacology of CD123 NKCE, were observed at CD123 NKCE concentrations at and above 0.0025 μg/mL. As expected, no decrease in the number of CD123+ cells were observed with the humanized anti-CD52 IgG1, despite the clear increase in multiple cytokines. In comparison, the CD3-CD123 TCE tool compound demonstrated a high fold increase for all cytokines measured, and a decrease in CD123+ cells of ˜20% at the single concentration tested (10 μg/mL), demonstrating that engagement of NK-cells in concert with CD123, rather than T-cells, results in much less cytokine release and more effective reduction of CD123+ cells (˜50%) under the conditions of this study.

In summary, the CD123 NKCE was evaluated in a human in vitro WB-AQ CRA and resulted in mean increases of IL-6 (13- to 21-fold) and IFNγ (10- to 24-fold) at and above the concentration of 0.25 μg/mL and induced the pharmacodynamic effect of a reduction in CD123+ cells, in whole blood from 13 healthy human donors, consistent with engagement of NK-cells and the expected pharmacology of CD123 NKCE. Based on these results, CD123 NKCE demonstrates a low risk for inducing CRS in humans.

### Example 6. Exploratory Repeat-Dose (Once Weekly for 4 Weeks) 1-Hour Intravenous Infusion Toxicity Study in NHPs with 3-Week Observation Period

The objective of this study was to determine the potential toxicity of CD123 NKCE when

administered once weekly to cynomolgus monkeys by 1-hour IV infusion for 4 weeks, and to assess the potential delayed onset toxicity and/or the reversibility of potential toxicity up to 4 weeks after the last administration. In addition, systemic exposure of CD123 NKCE, incidence of ADA was assessed and PD parameters (ie, CD123+ cell depletion and effects on NK-cells) were examined in the peripheral blood and in bone marrow by flow cytometry.

Cynomolgus monkeys (35 to 49 months of age; 2 animals per sex and per group) received an aqueous solution of CD123 NKCE at 100 or 3000 μg/kg/adm by 1-hour IV infusion, once weekly, for 4 weeks (on Days 1, 8, 15, and 22; dosing volume: 5 mL/kg). The parameters evaluated included mortality, clinical signs, body weight, injection site examination, body temperature, electrocardiography parameters, hematology, clinical chemistry (including C-reactive protein [CRP] and ferritin), coagulation, and urinalysis.

Blood was collected for TK evaluations, ADA, cytokines analysis (IL-2, IL-10, TNFα, IFNγ, IL-10, IL-6, and IL-8), and flow cytometry analysis.

Bone marrow samples were also collected for immune function evaluation by flow cytometry analysis.

One monkey per sex per dose was euthanized and necropsied at the end of the treatment (ie, 1-week after the last administration) and the remaining monkeys were euthanized and necropsied at the end of the 4-week recovery period. Organ weights were recorded and microscopic examinations were conducted.

Individual TK parameters of CD123 NKCE in plasma after a weekly repeat 1-hour IV infusion at 100 μg/kg/adm for 4 weeks are presented in Table 7.

TABLE 7
Individual (n = 2/sex) TK parameters of CD123 NKCE
in plasma after a weekly repeat 1-hour intravenous infusion
at 100 μg/kg/adm for 4 weeks.

| | Dose | | Animal | Cₘₐₓ | tₘₐₓ | AUC_{0-7 d} |
|---|---|---|---|---|---|---|
| Sex | (μg/kg/adm) | Day | ID | (ng/mL) | (day) | (day*ng/mL) |
| Male | 100 | 1 | 1 | 1750 | 0.04 | 720 |
| | | | 2 | 1520 | 0.06 | 1360 |
| | | 8 | 1 | 1340 | 0.04 | 901 |
| | | | 2 | 1332 | 0.21 | 281 |
| | | 15 | 1 | 19.3 | 0.04 | 1.07 |
| | | | 2 | 205 | 0.04 | 26.7 |
| | | 22 | 1 | 0.998 | 0.04 | 0.0314 |
| | | | 2 | <LLOQ | — | NC |
| Female | 100 | 1 | 3 | 1550 | 0.04 | 1000 |
| | | | 4 | 1870 | 0.04 | 1100 |
| | | 8 | 3 | 1980 | 0.04 | 1710 |
| | | | 4 | 1610 | 0.04 | 1520 |
| | | 15 | 3 | 256 | 0.04 | 217 |
| | | | 4 | 5.61 | 0.04 | 0.451 |
| | | 22 | 3 | <LLOQ | — | NC |
| | | | 4 | <LLOQ | — | NC |

Abbreviations:
<LLOQ: below the lower limit of quantification (LLOQ = 0.250 ng/mL);
NC: Not calculable

Individual TK parameters of CD123 NKCE in plasma after a weekly repeat 1-hour IV infusion at 3000 μg/kg/adm for 4 weeks are presented in Table 8.

TABLE 8
Individual (n = 2/sex) TK parameters of CD123 NKCE
in plasma after a weekly repeat 1-hour intravenous infusion
at 3000 μg/kg/adm for 4 weeks.

| | Dose | | Animal | Cₘₐₓ | tₘₐₓ | AUC_{0-7 d} |
|---|---|---|---|---|---|---|
| Sex | (μg/kg/adm) | Day | ID | (ng/mL) | (day) | (day*ng/mL) |
| Male | 3000 | 1 | 5 | 78600 | 0.06 | 241000 |
| | | | 6 | 72200 | 0.21 | 212000 |
| | | 8 | 5 | 92000 | 0.04 | 348000 |
| | | | 6 | 91900 | 0.21 | 272000 |
| | | 15 | 5 | 124000 | 0.04 | 254000 |
| | | | 6 | 33600 | 0.06 | 9460 |
| | | 22 | 5 | 61600 | 0.04 | 116000 |
| | | | 6 | 365 | — | 42.7 |
| Female | 3000 | 1 | 7 | 100000 | 0.04 | 247000 |
| | | | 8 | 48300 | 0.06 | 248000 |
| | | 8 | 7 | 137000 | 0.04 | 182000 |
| | | | 8 | 88300 | 0.21 | 234000 |
| | | 15 | 7 | 2310 | 0.04 | 229 |
| | | | 8 | 11900 | 0.04 | 1870 |
| | | 22 | 7 | <LLOQ | — | NC |
| | | | 8 | 149 | 0.04 | 9.17 |

Regardless of the dose level, maximal CD123 NKCE plasma concentrations were observed between the end of the 1-hour IV infusion and 5 hours after the start of each weekly infusion.

Regardless of the dose level, all CD123 NKCE-treated monkeys exhibited a positive status for ADA on Day 29, except for one animal (male No. 5) at 3000 μg/kg/adm where no ADA response was observed.

Overall, due to the presence of ADA, CD123 NKCE exposure were significantly reduced in all monkeys after the 3rd infusion, except for the animal (male No. 5) at 3000 μg/kg/adm, who exhibited a sustained exposure for 4 weeks, consistent with a negative ADA status throughout the study.

From 100 to 3000 μg/kg/adm after the first infusion on Day 1, CD123 NKCE exposure (AUC0-7d) was more than dose proportional with a 30-fold increase in dose (100 to 3000 μg/kg/adm) leading to greater than a 200-fold increase in exposure in male and female monkeys.

Overall, no accumulation was observed in animals on Days 8, 15, and 22 after a weekly repeat 1-hour IV infusion of CD123 NKCE at 100 or 3000 μg/kg/adm for 4 weeks, including the animal, presenting no ADA response. Regardless of the dose level, no sex effect was evidenced on Day 1 (other dosing days not evaluable due to the positive ADA status of animals).

CD123 NKCE was clinically well tolerated without any clinical signs, modification in body weight and body temperature, and effect on ECG throughout the study. No adverse CD123 NKCE-related changes were observed in clinical chemistry parameters, and only consisted of a minimal to slight decrease of urea values in all animals on Day 2 (return to normal on Day 29 or Day 50). No compound-related findings observed for hematology, coagulation, and urinalysis parameters were reported. CD123 NKCE was well tolerated at the injection site.

Very low IL-2, IL-6, and IL-10 cytokine release was observed after both doses in all treated animals, including the animal at 3000 μg/kg/adm, presenting a negative ADA status throughout the study and which is the animal with the highest exposure to CD123 NKCE. Changes in IL-6 (<200 μg/mL) were observed from 100 μg/kg/adm after each dosing and those in IL-2 (<10 μg/mL) and IL-10 (<60 μg/mL) observed at 3000 μg/kg/adm mainly after the 3rd and the 4th dosing. TNFα, IFNγ, IL-10, and IL-8 were found below lower limit of quantification (LLOQ) in all animals.

Immunophenotyping using flow cytometry showed similar effects on the CD123-positive cell and NKp46-positive NK cell populations in blood and in bone marrow at both doses tested. To be noted that under the study conditions, i) no results were interpreted for CD123-positive monocytes and NK cells that expressed CD107a due to absence or low cell counts in whole blood and in bone marrow; and ii) the cells number level of NK cells that expressed PD1 and CD69 was very low and therefore could not considered as a relevant activation.

The effects on immune cells were consistent with the expected PD effects of CD123 NKCE, mainly characterized by a CD123-positive cells depletion and a NKp46-positive NK cells decrease. The changes in blood consisted of i) a decrease of CD123-positive cells observed at 1.5 hours after the 1st administration up to 24 hours after the 3rd administration in all monkeys (except for male No. 1 at 100 μg/kg/adm) with a rebound (above baseline) observed on Day 22/29 in all monkeys (except male No. 5, top dose male without ADA exposed up to the end of dosing, where the rebound was observed at the next timepoint analyzed, 4 weeks after the last (4th) administration); and ii) a decrease of NKp46-positive NK cells observed at 1.5 hours up to 72 hours after the 1st administration, and 24 hours after the 2 nd administration in all monkeys. This decrease was also observed at 1.5 hours after the 3rd administration in all monkeys, and at 24 hours after the last (4th) administration in male No. 5. The changes in bone marrow consisted of i) a decrease of CD123-positive cells observed 24 hours after the 2nd administration for all monkeys with a rebound (above baseline) observed at the next timepoint analyzed (one week after the last (4th) administration) for all monkeys (except for monkeys No. 1 and 2 at 100 μg/kg/adm); and, ii) a decrease of NKp46-positive NK cells observed 24 hours after the 2nd administration for all monkeys with a return to baseline at Day 29 (for most of monkeys) and at Day 50 for male No. 5.

The examination of the hematoxylin-eosin-stained tissues and/or organs showed microscopic findings limited to the bone marrow collected from the sternum and the stomach. The microscopic findings in the bone marrow consisted of a minimal to slight increased myeloid/erythroid (M/E) ratio, characterized by minimally increased number of myeloid cells and decreased number of adipocytes, without atypia feature. No differences in CD123 immunostainings in the sternal bone marrow were seen between treated NHP or stock control NHP. Since the classic source of hematopoietic stem cells is the bone marrow, and the study is conducted in healthy animals, our hypothesis is that the decrease of CD123+ cells induced by CD123 NKCE resulted in a feedback mechanism that led to a stimulation of the bone marrow. Stomach lesions without atypia or ulceration features were noted in a few animals at both doses and were characterized by a minimal multifocal glandular single cell necrosis in the mucosa of the fundus/pylorus in one animal of each dose, 1 week after the last (4th) injection, and in both male and female at 3000 μg/kg/adm, 4 weeks after the last (4th) injection, and a slight to moderate diffuse mononuclear inflammatory cell infiltration (constituting of a mixed cellular population of CD3, CD8, CD68, CD56 and CD123 cells) revealed by IHC staining in the mucosa. Such findings are commonly observed in control cynomolgus monkeys, which have some degree of stomach inflammation/inflammatory cell infiltration as a background finding. Special stains evaluation (Warthin-Starry, Gram, PAS and Toluidine blue) did not reveal any increase in bacterial and/or fungal opportunistic populations when compared to what is generally observed in control NHP.

The small group size of animals and the absence of concurrent controls in the study made it difficult for a clear interpretation. Regardless, the gastritis associated with the minimal glandular hyperplasia was considered as non-adverse due to its minimal severity and the absence of associated clinical pathology parameter changes. The possible relationship to CD123 NKCE was assessed in the GLP toxicity study, with higher number of animals including controls.

### Example 7. GLP Repeat-Dose (Twice Weekly for 5 Weeks) 1-Hour Intravenous Infusion or Subcutaneous Toxicity Study in Cynomolgus Monkeys with a 6-Week Recovery Period

The objective of this study was to determine the potential toxicity of CD123 NKCE when given twice weekly for 5 weeks (9 administrations in total on Days 1, 4, 8, 11, 15, 18, 22, 25, and 29) by IV (1-hour infusion) or SC (bolus) route to cynomolgus monkeys. Assessments of delayed onset toxicity and/or reversibility of toxicity were made during 1-week observation and 6-week recovery periods. The TK characteristics of CD123 NKCE, ADA, and PD (CD123+ cell depletion and effects on NK-cells in peripheral blood and bone marrow) profiles of CD123 NKCE were also assessed.

Cynomolgus monkeys (5 per sex per dose) received the control article (10 mM Histidine buffer pH 6.0/Sucrose 8% [w/v]/PS80 [w/v]/EDTA 10 μM subcutaneously (bolus, 2.7 mL/kg) followed by IV dosing (1-hour infusion, 5 mL/kg), or a solution of CD123 NKCE at doses of 100 and 3000 μg/kg/adm by IV (5 mL/kg) or 3000 μg/kg/adm by SC (2.7 mL/kg), respectively, on Days 1, 4, 8, 11, 15, 18, 22, 25, and 29.

Three monkeys per sex per dose were euthanized and necropsied 1-week after the last administration (Day 36) and the remaining 2 monkeys per sex per dose were euthanized and necropsied at the end of the 6-week recovery period (Day 71/72).

The following parameters and endpoints were evaluated in this study: clinical signs, local tolerance, body weight, food consumption, body temperature, neuro-behavioral evaluation, ECG, blood pressure, respiration, ophthalmology, clinical pathology parameters (hematology, coagulation, clinical chemistry, and urinalysis), TK parameters, ADA evaluation, cytokine determination, whole blood and bone marrow immunophenotyping, macroscopic observations, organ weights, and histopathologic findings.

The TK of plasma CD123 NKCE was evaluated on Days 1, 4, 8, and 29, and summary CD123 NKCE TK parameters in plasma are presented in Table 9.

TABLE 9
Mean (% CV, n = 5/sex/group) CD123 NKCE toxicokinetic parameters
on Days 1, 4, 8, and 29 in cynomolgous monkey plasma following
twice weekly 1-hour IV infusion (100 and 3000 μg/kg/adm) or
SC administration (3000 μg/kg/adm) of CD123 NKCE for 5 weeks.

| | | Dose | | Cₘₐₓ | Tₘₐₓ | AUC_{(0-3 d)} | AUC_{(0-4 d)} |
|---|---|---|---|---|---|---|---|
| Route | Sex | (μg/kg/adm) | Day | (ng/mL) | (day) | (day*ng/mL) | (day*ng/mL) |
| IV | M | 100 | 1 | 2080 | 0.042 | 1490 | NA |
| | | | 4 | 2100 | 0.042 | 3440 | 3760 |
| | | | 8 | 2130 | 0.042 | 3130 | NA |
| | | | 29 | 1280 | 0.042 | 2070 | NA |
| | | 3000 | 1 | 87600 | 0.042 | 128000 | NA |
| | | | 4 | 108000 | 0.042 | 225000 | 276000 |
| | | | 8 | 128000 | 0.042 | 261000 | NA |
| | | | 29 | 10.2 | 0.042 | NC | NA |
| | F | 100 | 1 | 2080 | 0.042 | 1670 | NA |
| | | | 4 | 23400 | 0.042 | 3630 | 3920 |
| | | | 8 | 2210 | 0.042 | 3150 | NA |
| | | | 29 | 6.21 | 0.042 | NC | NA |
| | | 3000 | 1 | 81700 | 0.042 | 140000 | NA |
| | | | 4 | 117000 | 0.042 | 246000 | 299000 |
| | | | 8 | 114000 | 0.208 | 257000 | NC |
| | | | 29 | 163 | 0.042 | 71.5 | 17.5 |
| SC | M | 3000 | 1 | 27700 | 3 | 61100 | NA |
| | | | 4 | 38400 | 0.208 | 122000 | 167000 |
| | | | 8 | 58600 | 0.208 | 167000 | NA |
| | | | 29 | 16.8 | 0.208 | 42.9 | NA |
| | F | 3000 | 1 | 24800 | 2 | 60300 | NA |
| | | | 4 | 31900 | 0.208 | 101000 | 139000 |
| | | | 8 | 52200 | 0.208 | 140000 | NA |
| | | | 29 | 15.7 | 0.208 | NC | NA |

Regardless of the dose level, route of administration or sex, most of the CD123 NKCE-treated animals screened ADA positive on Day 15 (26/30 animals) and all CD123 NKCE-treated animals exhibited a positive screening status for ADA on Day 36. Overall, the data suggest that the presence of ADA in all animals had a notable impact on the plasma concentrations, with CD123 NKCE plasma concentrations generally starting to decrease or to be no longer quantifiable by Day 15. Almost all CD123 NKCE plasma concentrations were below the quantitation limit (<5.00 ng/mL) on Day 29. there was only one male (100 μg/kg/adm IV) that screened ADA positive with comparable levels of CD123 NKCE in plasma between Day 29 and Day 1. Due to the presence of ADA impacting exposure, the dose proportionality, sex effect, and bioavailability could not be evaluated on Day 29.

Following IV infusion, exposure to CD123 NKCE (mean AUC(0-3d) and AUC(0-4d)) increased with increasing dose in a greater than dose proportional manner from 100 to 3000 μg/kg/adm in males and females on Days 1, 4, and 8. Mean AUC(0-3d) or AUC(0-4d) values increased from 73.4- to 86.0-fold on Days 1, 4, and/or 8 in males and females, over a 30-fold dose range.

Following repeated IV infusion or SC administration of CD123 NKCE, the mean AUC(0-3d) was generally slightly higher on Day 4 or 8 than on Day 1 (about 1.7- to 2.7-fold higher). On Day 29, due to the marked decrease of CD123 NKCE plasma concentrations, which was probably related to the presence of ADA, most of the TK parameters could not be reported, and when reportable, the maximum concentration (Cmax) and AUC(0-3d) were generally notably lower on Day 29 than when compared to Day 1. The only exception was one male at 100 μg/kg/adm IV, which screened ADA positive, and for which exposure was comparable between Day 1 and Day 29.

No sex effect was observed between male and female exposure on Days 1, 4, and 8 across the dose levels or routes of administration.

Following SC administration of CD123 NKCE at 3000 μg/kg/adm, the mean bioavailability of CD123 NKCE (assessed by mean AUC(0-3d)) ranged from 41.2% to 63.8%, in males and females on Days 1, 4, and 8.

There was no mortality during the study. CD123 NKCE was locally well tolerated whatever the route of administration and dose level. There were no CD123 NKCE-related clinical signs, and no effects on body weight, food consumption, and clinical pathology parameters, and no findings were noted at ophthalmological evaluation in any group.

No acute or long-term CD123 NKCE-related effects were noted on neuro-behavior, body temperature, arterial blood pressure, quantitative and qualitative ECG parameters, or respiratory rate, evaluated on Days 4 and 25 in any group. Noteworthy effects on IL-1Ra, IL-10, TNFα, and MIP1β levels were only noted at 3000 μg/kg/adm when given IV and consisted of minimal to moderate increases in IL-1Ra, IL-10, and MIP1β, 1 or 5 hours after the 5th and 9th infusion and very minimal increase in TNFα, 1 hour after the 9th infusion. Maximal concentrations were reached 1 hour (IL-10, TNFα, and MIP1β) and 5 hours (IL-1Ra) after the 9th dosing. Considering the magnitude of changes and levels back to baseline 5 or 24 hours, the changes were considered as non-adverse.

Changes in total CD123+ cell populations and NK cells that expressed NKp46 were observed in blood and bone marrow and consisted of (i) a CD123 NKCE-related decrease of total CD123+ cell numbers for all treated groups after the 1st administration up to 24 hours after the 3^{rd} administration in blood, and 24 hours after the 3rd administration in bone marrow, followed by a CD123 NKCE-related increase in blood and bone marrow, and a return to baseline at the end of the

6-week recovery period in both blood and bone marrow; and (ii) a CD123 NKCE-related decrease of NKp46+NK cells number after the 1st administration up to 24 hours and after the 3^{rd} administration in the blood; this decrease was followed by a CD123 NKCE-related increase for the IV doses groups and a return to baseline at the end of the 6-week recovery period; in bone marrow, no CD123 NKCE-related effect was observed on NKp46+NK cells.

At the terminal euthanasia (Day 36, ie, one week after the last 9th administration) and at the end of the 6-week recovery period, there were no CD123 NKCE-related organ weight, macroscopic or microscopic findings. Inflammatory cell infiltrates in the gastric mucosa of mild severity were observed in few animals including a control male; these changes, known to occur commonly in untreated cynomolgus monkeys (20, 21, 22), were considered within the background range variation and unrelated to the administration of the compound.

The NOAEL was set at 3000 μg/kg/adm by the IV and SC route. The mean AUC (0-3d) values (after the 3rd administration) were for males 261 000/167 000 day·ng/mL following IV/SC administration, and for females 257 000/140 000 day·ng/mL, respectively.

### Example 8. Study of CD123 NKCE in Patients with Relapsed or Refractory (r/r) AML, B-ALL, BPDCN, or High-Risk Myelodysplastic Syndrome

This is a Phase 1/2, open label, first in-human study to determine the maximum tolerated or maximum administered doses, and to evaluate the safety, tolerability, pharmacokinetics, pharmacodynamics, and anti-tumor activity of CD123 NKCE as describe herein in various hematological malignancies, in adult or pediatric patients.

### Study Overview:

This is a first-in-human, open-label Phase 1/2 dose-escalation/expansion Phase 1/Phase 2 study to determine the maximum tolerated or maximum administered doses, and evaluate the safety, tolerability, pharmacokinetics, pharmacodynamics, and anti-tumor activity of CD123 NKCE administered intravenously as a single agent in participants aged ≥12 years with relapsed or refractory AML (r/r AML), B-ALL, or HR-MDS, or pediatric patients with r/r AML, BPDCN, or B-ALL.

### Rationale

There is growing evidence to suggest that AML is derived from leukemia stem cells (LSCs) that give rise to leukemic blasts in in vitro and in vivo models. It is hypothesized that the persistence of LSCs causes relapse after an initial remission. Thus, the eradication of LSCs may be a requirement for a cure and is an important therapeutic goal. One potential therapeutic target in AML is the surface receptor CD123, the interleukin (IL)-3 receptor alpha chain (IL-3Rα) that is expressed at high levels on LSCs. CD123 is overexpressed in AML blasts and on CD34-positive/CD38-negative AML leukemic stem cells, while other hematologic malignancies (including MDS and B-ALL) have also been shown to be CD123 positive. In normal peripheral blood, CD123 exhibits a varying level of expression, with the highest density on plasmacytoid dendritic cells and basophils, and intermediate levels on myeloid dendritic cells, monocytes, eosinophils and naïve B cells.

The proposed study is a first-in-human, dose finding study to determine the safety, pharmacokinetic (PK), pharmacodynamic (PD), and preliminary efficacy of CD123 NKCE administered intravenously (IV) to adults and adolescents with CD123-expressing hematologic malignancies who have relapsed or failed prior treatment and have no alternative standard treatment options.

This is a first in human (FIH) Phase 1 dose escalation study designed to identify the optimal dose and regimen (recommended dose for expansion, RDE) of CD123-NKCE that can be safely administered in patients with AML. The proposed starting dose of 10 μg/kg is based on the mechanistic Pharmacokinetic (PK)/Pharmacodynamic (PD) model and considers the estimated exposure in human (Cmax, AUC0-3d) equivalent to 1/10 (safety factor) the exposure quantified in non-human primate NHP at the No Observed Adverse Effect Level (NOAEL) defined at 3000 μg/kg/adm after 5-weeks twice-weekly dosing.

### Study Duration

The expected duration of this Phase 1/2 study is about 2.5 years for a participant, provided the participant does not meet the criteria for stopping treatment with the investigational medicinal product (IMP). The study duration includes:
a screening period of up to 2 weeks (14 days) in escalation/expansion (except 28 days for MDS Cohort B),
an induction treatment duration of 3 months (3 cycles of 28 days each), a maintenance treatment duration of 2 years (13 cycles of 56 days each) and
an end of treatment (EOT) visit (30 days±7 days) after last investigational medicinal product (IMP) administration. If discontinuation occurs during maintenance, EOT is preferred within 30 days (+/−7 days) but must be performed within 60 days (+/−7 days) of the last IMP administration or prior to initiation of further therapy, whichever comes first.

Survival status of each participant will also be collected during the Follow-Up Period that will occur every 2 months±14 days to assess survival status. The study completion date will be when the last participant in the Expansion Part has been followed for 12 months or until death (whichever occurs first), to document prolonged remission and survival rate at 1 year.

A trial participant will receive 3 induction cycles even if CR, CRi or CRh is achieved prior to completion of 3 cycles (28 days per cycle). An increase or a decrease in the number of induction cycles may be recommended by the Study Board based on evaluation of available safety data including DLTs, AEs, SAE, clinical PK and PD. If CR, CRi or CRh (AML participants) or CR, CR equivalent or PR (MDS participants) is not achieved within 3 induction cycles, induction therapy must continue until CR, CRi or CRh (AML participants) or CR, CR equivalent or PR (MDS participants) is achieved (up to 6 cycles maximum). Once a trial participant has achieved CR, CRi or CRh, the trial participant must continue with maintenance cycles (56 days per cycle) up to a total of 13 cycles. A participant who achieves an adequate response to treatment with CD123 NKCE and meets other criteria required to undergo hematopoietic stem cell transplantation (HSCT) may proceed to HSCT after discontinuation of CD123 NKCE. If a participant discontinues CD123 NKCE to undergo HSCT, but is then deemed ineligible for HSCT, the participant may restart treatment with CD123 NKCE at the originally scheduled DL and dose.

### Stopping Criteria

A trial participant must discontinue treatment with CD123 NKCE if the participant develops evidence of an unacceptable adverse event, or disease progression, or the trial participant decides to stop treatment with CD123 NKCE.

### Stopping Rules:

“Excessive toxicities” are defined as deaths (other than death related to PD) within 30 days of therapy or Grade 4 treatment emergent adverse events (TEAEs). Bayesian toxicity monitoring is used to determine the stopping boundaries based on the following assumptions: maximum probability of excessive toxicities allowed=0.2, prior distribution for excessive toxicity rate: Beta (1,1), minimum number of participants before early stopping rule applies=6, cohort size=1, and posterior probability of excessive toxicity rate >0.2 is at least 80%. Table 21 shows the number of participants with excessive toxicities observed before enrollment in the trial will be paused until an appropriate evaluation of the cause of death and toxicity is conducted by the Study Board and a correction plan is established.

TABLE 9B
Bayesian toxicity monitoring plan in dose expansion phase.

| N | 6 | 7-10 | 11-14 | 15-19 | 20-23 | 24-27 | 28-31 | 32-36 | 37-40 |
|---|---|---|---|---|---|---|---|---|---|
| n | ≥2 | ≥3 | ≥4 | ≥5 | ≥6 | ≥7 | ≥8 | ≥9 | ≥10 |

Abbreviations:
N = total number of participants evaluated; n = number of participants with excessive toxicities.

### Study Interventions

### Investigational Medicinal Products (AxMP):

Formulation: The CD123 NKCE drug product is presented as a powder in solution for infusion in vials containing 100 mg/vial.
Route(s) of administration: IV infusion with specific instructions on the preparation and administration of CD123 NKCE provided in the Pharmacy Manual.
Dose regimen:
   Induction: Depending on the dose level, CD123 NKCE will be administered twice-weekly and/or weekly during Cycle 1. In subsequent induction cycles (2+), CD123 NKCE will be administered once-weekly, i.e., on D1, D8, D15, D22 of a 28-day cycle. Participants will receive at minimum 3 induction cycles prior to switching to maintenance treatment.
      Adult escalation: CD123 NKCE is administered on Day 4 and Day 11 only in Cycle 1 and for lower dose levels that require twice-weekly administration within the first 2 weeks, for Cycle 1 administration on D1, D4, D8, D11, D15, and D22. CD123 NKCE is administered once-weekly on D1, D8, D15, D22 at Cycle 1 and all other cycles.
      Adult expansion: CD123 NKCE is administered on D1, D8, D15, and D22 for all cycles.
      Pediatric escalation/expansion: CD123 NKCE is administered on D1, D8, D15, and D22 for all cycles.
   Maintenance (Adult and Pediatric escalation/expansion): Once a participant achieves CR, CRh or CRi, CD123 NKCE will be administered by IV on D1 and D29 of a 56-day cycle for up to 13 cycles. Based on emerging clinical data, the frequency of the IMP treatment may be adjusted in the maintenance period. As additional clinical data emerges, the maintenance dosing schedule within the maintenance cycle may be further adjusted to potentially include more or less frequent dosing within the cycle.
The IV dosing regimen will be weight-based, however, if a participant weighs >133 kg, the participant's weight will be capped at 133 kg for dose calculations.

### Non-Investigational Medicinal Products:

Participants will receive the following premedication to prevent or reduce the severity of potential acute adverse events, prior to CD123 NKCE administration, for the first Cycle:
Acetaminophen (paracetamol) 650 to 1000 mg oral route (PO preferred, but IV acceptable) or adult/pediatric equivalent, and then optionally thereafter as needed.
Diphenhydramine 25 to 50 mg IV 30-60 minutes prior to administration of CD123 NKCE (IV preferred but PO is also acceptable when IV formulation is not available) or the pediatric equivalent (e.g., cetirizine, promethazine, dexchlorpheniramine, according to local approval and availability), and then optionally thereafter as needed. Premedication may be optional from Cycle 2 as described below:
   For participants who have no CRS or IRR during the first Cycle: premedication for the subsequent infusions is optional at the Investigator's discretion. However, if during subsequent infusions without premedication the participant experiences a CRS/IRR of any grade, premedication must be restarted for all subsequent infusions.
   If a participant develops a CRS/IRR Grade <3 during the first Cycle only, and then experiences no further CRS/IRR during their 2^{nd} cycle, the Investigator may consider omitting premedication for the next cycle. If no CRS/IRR is observed during the next cycle without premedication, premedication is optional for the subsequent cycles at the discretion of the investigator. However, if during the next cycle without premedication the participant experiences a CRS/IRR of any grade, premedication must be restarted for all subsequent cycles.

### Follow-Up

All ongoing related adverse events or newly reported related adverse events, and all SAEs regardless of causal relationship are to be followed at least every 30 days until resolution or stabilization.

The first cut-off date will be at the end of the first cycle of the last participant treated and

evaluable for DLT in the Dose Escalation Part, in order to evaluate data from at least the first cycle for all participants when determining the MTD, MAD, and preliminary RDE.
The second and subsequent cut-off dates will be when the last participant in the Expansion/Optimization cohort A, cohort B or cohort D has completed 3 cycles of induction, has early progression, or has reached CRc (for AML) or has reached CR, CR equivalent, PR, CRL, CRh, or HI (for HR-MDS), whichever occurs first, in order to assess malignancy response. Different data extraction dates may apply depending on the timing of adult and pediatric cohort enrollments and data maturity. After the second cut-off date, participants can continue to receive IMP, if clinical benefit is observed, until permanent discontinuation criteria are met (personal choice, detrimental effects, no remission, pregnancy, or too low body mass) and will continue to undergo all assessments as per the study schedule of activities. The study completion date will be when the last participant in the Expansion Part has been followed for 12 months or until death (whichever occurs first), to document prolonged remission and survival rate at 1 year.

### Statistical Considerations:

Analysis of primary endpoints
   Dose escalation and dose expansion/optimization cohort C: DLTs will be summarized by dose level and overall. Details of DLTs will be provided by participant.
   Dose expansion/optimization part cohort A, B and D: For Cohort A and D, CRc rate (for R/R AML) will be summarized with descriptive statistics. For Cohort B, overall response rate will be summarized with descriptive statistics. A 90% two sided confidence interval will be computed using Clopper-Pearson method.
Analysis of secondary endpoints
   Dose escalation and dose expansion/optimization cohort C: rate of CR+CRh+CRi for R/R AML and B-ALL, CR rate for HR-MDS in dose escalation, ORR rate for HR-MDS in dose expansion/optimization cohort C, and CR+clinical CR rate for BPDCN (if applicable) will be summarized with descriptive statistics with 90% two-sided confidence interval using Clopper-Pearson method.
   Dose escalation and expansion/optimization: the treatment-emergent AEs/SAEs and laboratory abnormalities during the on-treatment period, as well as PK profile characterization will be summarized using descriptive statistics by dose level (for escalation) and by cohort (for expansion), and overall (if applicable). PK profile characterization and immunogenicity will be assessed by dose level/cohort and overall.
   Dose expansion/optimization part cohort A, B and D: For Cohort A and D, rate of CR+CRh, rate of CR+CRh+CRi+MLFS+PR, and rate of subsequent HSCT for AML will be summarized with descriptive statistics. A 90% two-sided confidence interval will be computed using Clopper-Pearson method. Time-to-event endpoints such as duration of different types of responses (CR+CRh+CRi; CR+CRh; and CR+CRi+CRh+MLFS+PR), EFS, TTF and OS will be summarized with Kaplan-Meier method. For Cohort B, alternative CR rate and rate of subsequent HSCT will be summarized with descriptive statistics. A 90% two-sided confidence interval will be computed using Clopper-Pearson method. Time-to-event endpoints such as duration of ORR, duration of alternative CR, OS, EFS, TTF, PFS will be summarized with Kaplan-Meier method.

### Objectives and Endpoints

TABLE 10

| Objectives | Endpoints |
|---|---|
| Primary | |
| Dose escalation: | |
| Adults: To determine the maximum tolerated | Incidence of dose-limiting toxicity (DLT), |
| dose (MTD) or maximum administered dose | occurring during Cycle 1 (Day 1 to Day 28) |
| (MAD) and Recommended Dose(s) for | |
| Expansion (RDEs) of CD123 NKCE | |
| administered as a single agent in participants | |
| with relapsed or refractory acute myeloid | |
| leukemia (r/r AML), high risk | |
| myelodysplastic syndrome (HR-MDS), or B- | |
| cell acute lymphoblastic leukemia (B-ALL). | |
| Pediatrics: To determine the maximum | |
| tolerated dose (MTD) or maximum | |
| administered dose (MAD) and RDEs of | |
| CD123 NKCE administered as a single agent | |
| in participants with r/r AML, blastic | |
| plasmacytoid dendritic cell neoplasm | |
| (BPDCN), or B-ALL. | |
| Expansion Cohort C: To confirm the | AML: Composite Complete Remission |
| tolerability of CD123 NKCE in Japanese | (CRc) rate: proportion of participants who |
| participants. | have a CR (complete remission) + CRh (CR |
| Expansion/Optimization: (Cohorts A, B, and | with partial hematologic recovery) + CRi |
| D) To assess the anti-cancer activity of | (complete remission with incomplete |
| CD123 NKCE administered as a single agent | hematologic recovery) according to the |
| at the preliminary Recommended Dose(s) for | modified AML IWG 2003 response criteria |
| Expansion (RDEs) and confirm the RDE in | MDS: Overall response rate (CR + CR |
| participants with r/r AML and MDS. | equivalent + PR + CRL + CRh + HI) − IWG |
| | 2023 MDS response criteria |
| Secondary (Adult and Pediatric) | |
| To identify the preliminary RDEs | Overall safety and tolerability, |
| (Escalation part) and confirm the RDE | pharmacokinetics (PK), pharmacodynamics |
| (Expansion/Optimization part; cohorts A, B, | (PD), preliminary anti-cancer activity |
| and D) | |
| To characterize the overall safety and | Type, frequency, severity, seriousness, and |
| tolerability profile of CD123 NKCE | relationship to CD123 NKCE of adverse |
| (Escalation and Expansion/Optimization) | events or abnormalities of laboratory tests or |
| | investigations; drug discontinuation due to |
| | adverse events |
| To characterize the PK profile of CD123 | PK parameters of CD123 NKCE (at least |
| NKCE when administered as a single agent | C_{trough}) |
| (Escalation and Expansion/Optimization) | |
| To evaluate the potential immunogenicity of | Incidence of anti-drug antibody (ADA) |
| CD123 NKCE (Escalation and | |
| Expansion/Optimization) | |
| To assess preliminary evidence of | AML: Rate of CR + CRh + CRi per AML |
| hematologic response (except BPDCN) | 2003 |
| (Escalation and Expansion/Optimization | modified International Working Group |
| Cohort C) | (IWG) |
| | response criteria |
| | MDS: CR rate per IWG 2006 MDS |
| | response |
| | criteria for escalation part and ORR rate per |
| | IWG 2023 MDS response criteria for |
| | expansion/optimization cohort C |
| | B-ALL: Rate of CR + CRh + CRi as defined |
| | by |
| | NCCN |
| To assess additional parameters of CD123 | Overall response rate defined as proportion |
| NKCE antitumor activity in AML | of participants who have a CR or Cri or CRh |
| participants at the RDE | or PR or MLFS (morphological leukemia- |
| (Expansion/Optimization part; cohorts A and | free state) according to the modified AML |
| D) | IWG response criteria. |
| | Duration of CR + CRh + Cri (Duration of |
| | CRc) defined as the time interval from the |
| | first documented evidence of CRc (CR, CRh |
| | or CRi) until disease relapse or death due to |
| | any cause, whichever comes first. |
| | Duration of CR + Cri + CRh + PR + MLFS |
| | (Duration of overall response rate) defined as |
| | the time from the first documented evidence |
| | of CR or Cri or CRh or PR or MLFS until |
| | disease relapse or death due to any cause, |
| | whichever comes first. |
| | Alternative CR rate defined as proportion of |
| | participants with CR + CRh (complete |
| | remission with partial hematological |
| | recovery) |
| | Duration of CR + CRh (Duration of |
| | alternative CR) defined as the time from the |
| | first documented evidence of CR or CRh |
| | until disease relapse or death due to any |
| | cause, whichever comes first. |
| | Event-free survival (EFS) defined as the time |
| | interval from the first day of treatment |
| | assignment to the date of earliest evidence of |
| | relapse, treatment failure, or death. |
| | Overall survival defined as time interval |
| | from the first day of treatment assignment to |
| | death from any cause. |
| | Rate of hematopoietic stem cell |
| | transplantation (HSCT) procedures |
| | immediately following study treatment |
| | administration but prior to subsequent |
| | therapy for treatment of AML. |
| | Time to treatment failure (TTF) defined as |
| | the time from first day of treatment |
| | assignment to discontinuation for any reason |
| | excluding remission, e.g., relapsed disease, |
| | refractory disease, participant preference or |
| | death. |
| To assess transfusion independence rate (TI) | Rate of conversion from transfusion |
| after treatment with CD123 NKCE at the | dependence during 56-day post-baseline |
| RDE (Expansion/Optimization, Cohorts A | period |
| and D) | |
| | Rate of participants who are transfusion |
| | independent at baseline and remain |
| | independent during 56-day post-baseline |
| | period. |
| To assess additional parameters of CD123 | Alternative CR rate defined as proportion of |
| NKCE anti-cancer activity in MDS | participants with CR, CR equivalent, CRuni, |
| participants at the RDE | CRbi, and CRh. |
| (expansion/optimization, Cohort B) | |
| | Duration of ORR: Defined as the time |
| | interval from the first documented evidence |
| | of CR, CR equivalent, CR_{L}, CRh, PR or HI |
| | to PD or relapse from CR, CR equivalent, |
| | CR_{L}, CRh, PR or HI as per 2023 IWG |
| | recommendations or death due to any cause, |
| | whichever comes first. |
| | Event-free survival (EFS): Measured from |
| | the first day of treatment assignment to the |
| | date of the first of the following events: PD, |
| | Failure to achieve CR (or CR equivalent), |
| | PR, CR_{L}, CRh, or HI within 6 months of |
| | study entry. Relapse from CR (or CR |
| | equivalent), PR, CR_{L}, CRh, or HI. Death |
| | from any cause. Patients not known to have |
| | any of these events are censored on the last |
| | date they were known not to have any of |
| | these events. |
| | Overall survival defined as time interval |
| | from the first day of treatment assignment to |
| | death from any cause. |
| | Rate of hematopoietic stem cell |
| | transplantation (HSCT) procedures |
| | immediately following study treatment |
| | administration but prior to subsequent |
| | therapy. |
| | Time to treatment failure (TTF) defined as |
| | the time interval from first day of treatment |
| | assignment to discontinuation for any reason |
| | excluding remission, e.g., relapsed disease, |
| | disease progression, unacceptable AE, |
| | participant preference, or death. |
| | Duration of alternative CR (CR + CR |
| | equivalent + CRL + CRh): the time interval |
| | from first documented evidence of CR, CR |
| | equivalent, CRL or CRh until PD or relapse |
| | from CR, CR equivalent, CRL or CRh as per |
| | 2023 IWG recommendations or death due to |
| | any cause, whichever comes first. |
| | Progression free survival (PFS): defined as |
| | the tiem interval from the first day of |
| | treatment assignment to the date of PD, |
| | relapse from CR (or CR equivalent), PR, |
| | CRL, CRh, or HI, death due to any cause, |
| | whichever comes first. |
| | Insert library content here if available |
| Exploratory | |
| Pediatrics only: To assess preliminary | Anti-leukemic activity as defined by |
| evidence of hematologic response | International Harmonization Project (IHP) |
| (escalation) | lymphoma criteria for BPDCN (modified) |
| | participants (e.g., ORR, clinical CR rate) |
| To monitor CD123 expression and kinetics | CD123 expression on various leukemic blast |
| of this expression under treatments | and non-blast populations in blood and bone |
| | marrow will be assessed by multiparameter |
| | flow cytometry and time course analysis of |
| | baseline and on therapy changes in CD123 |
| | receptor density. |
| To assess minimal residual disease (MRD) in | Minimal residual disease by molecular |
| participants achieving a CR, CRh, or Cri, and | biology assessment and/or flow cytometry |
| correlate MRD with clinical outcome (AML | will be assessed in bone marrow and/or blood |
| expansion/optimization only, Cohort A, C, | from participants achieving a CR, CRh, or |
| D) | Cri and correlated with clinical outcome. |
| To assess NK and T-cell subpopulations | Monitoring of NK and T-cell subpopulations |
| (e.g., CD4, CD8) and activation status | (e.g., CD4, CD8) and activation status in the |
| | peripheral blood and bone marrow aspirate. |
| To investigate the relationship between | Assessment of potential correlation between |
| CD123 expression, disease molecular | specific disease characteristics at baseline |
| subtype (as defined by marker expression, | (CD123 expression, molecular subtypes, |
| cytogenetics and/or | PIF/ER vs LR, cytogenetics, and/or |
| genomics/transcriptomics) baseline IgG, | genomics/transcriptomics), baseline IgG, |
| different AML subpopulations (i.e., PIF/ER | and clinical outcomes. |
| vs LR) and parameters of clinical response. | |
| To determine % reduction in CD123+ blasts | Assessment of the kinetics of treatment- |
| in the dose escalation and expansion | induced reduction in the % of CD123+ blasts |
| | as a pharmacodynamic marker for response |
| | to the IMP. |
| To assess levels of cytokines following | Assessment of plasma cytokine levels in |
| treatment administration, and their | blood at specific time points following |
| relationship with safety profile and clinical | treatment administration to evaluate |
| outcomes. | potential associations of cytokine levels with |
| | safety and clinical outcomes. |
| To assess the participant health-related | Assessment of the Quality of Life in Acute |
| quality of life (HRQL), disease and treatment | Myeloid Leukemia (AML-QOL) participant |
| related symptoms and impacts of symptoms | reported outcome instrument. |
| (Expansion/Optimization Phase Cohort A | |
| and C) | |
| | Patient Global Impression of Severity (PGIS) |
| | and Patient Global Impression of Change |
| | (PGIC) scales will be utilized as anchors to |
| | estimate/confirm established clinically |
| | meaningful change scores for AML- |
| | QOL/domain scores. |
| To assess qualitatively patient experience | Results from the Patient's Qualitative |
| with treatment (Expansion/Optimization | Assessment of Treatment version 2 (PQAT- |
| Phase Cohort A, B and C) | v2) will be used to assess participant- |
| | perceived advantages and disadvantages of |
| | treatment. |

### Participants

Escalation: CD123 will be administered to participants with r/r AML, HR-MDS, B-ALL, or BPDCN. It is anticipated that approximately 66 DLT-evaluable adult participants will be enrolled in the dose escalation part with expected assessment of about 11 main DLs while intermediate DLs may be investigated in case of dose or schedule modifications. Approximately 24 participants will be enrolled in the dose escalation part for pediatric with expected assessment of abut 3 main DLs while intermediate DLs may be investigated in case of dose or schedule modifications. The actual sample size will vary depending on DLTs observed, number of DLs actually explored and the number of participants enrolled that are not DLT-evaluable.

TABLE 11
Number of participants in the Escalation phase.

| | | | CD123 NKCE | |
|---|---|---|---|---|
| Escalation | | Target dose | Dose | No. Of Treated |
| Arm | Dose Level | (mg/kg) | (mg/kg) | participants |
| Adult Arm | DL1^{a} | 0.1 | 0.01, 0.03, 0.10 | 3 |
| | DL2^{a} | 0.3 | 0.03, 0.10, 0.30 | 4 |
| | DL0.75 mg^{b} | 0.75 | 0.75 | 6 |
| | DL3^{c} | 1 | 0.10, 0.30, 1 | 4 |
| | DL4^{c} | 1 | 0.30, 1 | 4 |
| | DL1 mg^{d} | 1 | 1 | 8 |
| | DL1.5 mg^{d} | 1.5 | 1.5 | 6 |
| | DL2 mg^{d} | 2 | 2 | 4 |
| | DL5^{c} | 3 | 1, 3 | 4 |
| | DL6^{d} | 3 | 3 | 8 |
| | DL7^{d} | 6 | 6 | 8 |
| Pediatric | DL1^{d} | 1 | 1 | 3 |
| Arm | DL1.5 mg^{d} | 1.5 | 1.5 | 2 |

^{a}LID: dosing on Day 1, Day 4, Day 8, Day 11, Day 15 then weekly during induction and every 4 weeks during maintenance
^{b}LID: dosing on Day 1, Day 4, Day 8 then weekly during induction and every 4 weeks during maintenance
^{c}LID: weekly dosing from first administration during induction then every 4 weeks during maintenance
^{d}Fixed weekly dosing from first administration during induction then every 4 weeks during maintenance
Abbreviations:
DL: dose level;
LID: lead-in-dose

The preliminary RDEs (pRDE) identified during the dose-escalation part may be further evaluated during the expansion/optimization part among the first 10-20 participants enrolled in the pRDE.

Expansion/Optimization: Approximately 100 r/r AML patients are expected to be enrolled and treated in Cohort A; up to 70 participants with HR-MDS are expected to be treated in Cohort B; up to 9 participants with HR-MDS, B-ALL or AML will be treated in Cohort C; up to 80 pediatric participants with r/r AML will be treated in Cohort D. Continuous safety and efficacy monitoring will be performed using Bayesian methods at informal interim analyses. The sample size for the dose expansion part is not based upon a power calculation but is intended to provide preliminary information on the activity of CD123 NKCE as a single agent in the r/r AML, HR-MDS adult and pediatric r/r AML populations.

Inclusion Criteria: Participants are eligible to be included in the study only if all of the following criteria apply:
A. Age
   Participant must be ≥1 year (except in France ≥2) old at the time the trial participant or legal guardian signs the informed consent form and will be assigned as follows:
a. Adult arm: aged ≥12 years old.
b. Pediatric arm: aged 1 (except in France 2) to 17 years old. Participants aged 12-17 years old may be enrolled in DL ≥3 as the second or subsequent participant in the DL, or in the ‘Pediatric arm’, when decided by the Study Board according to supporting data per protocol.

TABLE 12

| | Age | |
|---|---|---|
| Arm | group | Phases |
| Adult arm | ≥12 years | Escalation phase |
| | old | Expansion Cohort |
| | | A, B, (C is ≥18 years |
| | | old) |
| Pediatric | 1 (except | Escalation Phase |
| arm | in France | Expansion Cohort D |
| | ≥2) to 17 | |
| | years old | |

Participants aged 12-17 years old may be enrolled in DL ≥3 as the second or subsequent participant in the DL, or in the ‘Pediatric arm’, when decided by the Study Board according to supporting data per protocol.
B. Escalation and Expansion Cohorts a, C, and D (Adult and Pediatric)
   a. Confirmed diagnosis of primary or secondary AML [any subtype] according to World Health Organization (WHO) classification. Participants with AML must meet one of the following criteria, i), ii) or iii) and are limited to those with no available (or are ineligible) therapy with known clinical benefit.
      i. Primary Induction Failure (PIF) AML, defined as disease refractory to one of the following:
         1. An intensive induction attempt, per institution. Induction attempts include high-dose and/or standard-dose cytarabine±an anthracyclines/anthracenedione±an anti-metabolite, with or without growth factor or targeted therapy containing regimens. Examples include, but are not limited to: one cycle of high dose cytarabine (HiDAC) containing regimen; one cycle of liposomal cytarabine and daunorubicin; or two cycles of standard dose cytarabine containing regimen.
         2. For adults who are age 75 years or older, or who have comorbidities that preclude use of intensive induction chemotherapy; PIF is defined as AML refractory to one of the following less intensive regimens: 4 cycles of HMA or 2 cycles HMA+venetoclax.
      ii. Early relapse (ER) AML, defined as AML in relapse with CR, CRh or CRi duration <6 months on prior induction treatment.
      iii. Leukemia in first or higher relapse.
      iv. For participants aged 1 to 17 years old, primary induction failure is defined as disease refractory after two cycles of induction therapy according to Children's Oncology Group (COG) protocol guidelines.
   b. Adult Escalation and Adult Expansion Cohorts B, C only: Confirmed diagnosis of MDS, meeting with the following criteria:
      i. intermediate or high-risk category as per a Revised International Prognostic Scoring System (IPSS-R) AND
      ii. confirmed CD123+ expression status determined by local institutional standards AND
      iii. limited to those with no available (or are ineligible) therapy with known clinical benefit defined as:
         1. Not eligible for induction therapy and having completed ≥2 cycles of any of the following: hypomethylating agent (e.g., 5-azacitidine or decitabine) and/or venetoclax, chemotherapy, or targeted agents, or
         2. Not eligible for ASCT and having completed ≥1 course of induction therapy.
   c. Adult and Pediatric arms escalation and expansion Cohort C only: Confirmed diagnosis of CD123+B-ALL without extramedullary lesions that have no available (or are ineligible) therapy with known clinical benefit. Participants with non-CNS chloromatous disease are not allowed in the study.
C. Expansion (Adults only)
D. Participants' body weight: ≥10 kg.
E. Highly effective contraception: Contraceptive use by men and women should be highly effective consistent with local regulations regarding the methods of contraception for those participating in clinical studies.
   a. Male Participants: Are eligible to participate if they agree to the following during the study intervention period and for at least 3 months after study completion or the last dose of study treatment:
      i. Refrain from donating sperm; AND
      ii. Be abstinent from heterosexual intercourse as their preferred and usual lifestyle (abstinent on a long term and persistent basis) and agree to remain abstinent; or
      iii. Must agree to use contraception/barrier as detailed below:
         1. A mail condom and an additional highly effective contraceptive method when having sexual intercourse with a female of childbearing potential (FOCBP).
   b. Female Participants: Eligible to participate if she is not pregnant or breastfeeding, and one of the following conditions applies:
      i. Is a female of non-childbearing potential (FONCBP); OR
      ii. Is a female of childbearing potential and agrees to use a contraceptive method that is highly effective (with a failure rate of <1% per year), preferably with low user dependency or sexual abstinence if appropriate and barrier guidance during the study intervention period and for at least 3 months after the last administration of study intervention and agrees not to donate eggs (ova, oocytes) for the purpose of reproduction during this period. Contraception for female participants should start 2 weeks prior to C1D1.
      iii. A FOCBP must have a negative highly sensitive pregnancy test (urine or serum) as required by local regulations) within 7 days prior to the initial dose of study treatment before the first administration of study intervention.
         1. If a urine test cannot be confirmed as negative (e.g., an ambiguous result), a serum pregnancy test is required. In such cases, the participant must be excluded from participation if the serum pregnancy result is positive.
F. Informed Consent—Capable of providing signed informed consent, which includes compliance with the requirements and restrictions listed in the informed consent from (ICF) and in this protocol. In countries where legal age of majority is above 18 years, and for participants ≥12 and <18 years of age: the parent(s)/legal guardian(s) must provide written informed consent prior to any study-related procedures being performed. If the participant is also considered able by local regulation, consent from the participant will also be obtained before any study-related procedures are performed.
G. Pediatric arm and escalation only: Confirmed diagnosis of BPDCN according to World Health Organization (WHO) 2022 classification, who have relapsed or refractory disease with no available (or are ineligible) therapy with known clinical benefit.
H. Japan participants (Cohort C): Participant must be ≥18 years old at the time and the trial participant
Exclusion Criteria: Participants are excluded from the study if any of the following apply:
A. Eastern Cooperative Oncology Group (ECOG) performance status >2 (≥18 years-old). Karnovsky Scale (16-17 years-old)<50% or Lansky Scale (<16 years-old) <50%.
B. Ongoing or recent (within 5 years) evidence of significant autoimmune disease that requires or required treatment with systemic immunosuppressive treatments, which may suggest a risk for immune-related adverse events. The following are not exclusionary: vitiligo, childhood asthma that has resolved, residual hypothyroidism that required only hormone replacement or psoriasis that does not require systemic treatment.
C. History of an invasive malignancy that requires active therapy (adjuvant hormonal therapy is allowed) other than the one treated in this study, with the exception of resected/ablated basal or squamous-cell carcinoma of the skin or carcinoma in situ of the cervix, or other local tumors considered cured by local treatment.
D. Evidence of active central nervous system leukemia at the time of enrollment as evidenced by cytology or pathology. Central nervous system 1 disease (CNS1) and CNS2 disease according to COG classification are allowed. In the absence of clinical CNS disease, lumbar puncture is not required. History of CNS involvement is not exclusionary if CNS has been cleared with a documented negative lumbar puncture.
E. Known acquired immunodeficiency syndrome (AIDS-related illnesses) or HIV disease requiring antiretroviral treatment, or having active hepatitis B or C infection, or SARSCoV-2 infection. Participants with prior SARS-CoV-2 infection will be excluded if they have unstable post-COVID19 symptoms exclusion examples: unstable pulmonary disease, neurologic, gastrointestinal, and or vascular symptoms).
F. Prior/concomitant therapy:
   a. Prior treatment with an anti-CD123-directed agent.
   b. Prior HSCT with relapse beyond three months or prior CAR-T therapy in B-ALL with relapse beyond 2 months may be included only if off immunosuppression for a minimum of 4 weeks and no evidence of GvHD.
   c. Previous treatment with chemotherapy, radiotherapy, or immunotherapeutic agents within 21 days or 5 half-lives whichever is shorter prior to IMP administration (Cycle 1, Day 1). AEs related to prior therapy, except for alopecia, must have resolved to ≤Grade 1.
   d. Receiving at the time of first IMP administration corticosteroid as a concomitant medication with corticosteroid dose >10 mg/day of oral prednisone or the equivalent, except steroid inhaler, nasal spray, or ophthalmic solution.
   e. Requirement for tocilizumab for any other diagnosis within ≤14 days before the first administration of CD123 NKCE.
   f. Known contraindication to any of the non-investigational medicinal products (acetaminophen and diphenhydramine) without acceptable alternatives.
   g. AML, BPDCN, or HR-MDS participants with prior treatment with cellular therapy, e.g., chimeric antigen receptor T cell (CAR-T) or chimeric antigen receptor NK cell (CAR-NK). Prior CAR-T therapy is allowed for participants with B-ALL.
   h. Medical conditions requiring treatment with medications with narrow therapeutic index that are substrates of CYP enzymes and that cannot be closely monitored to allow for dose adjustment.
G. Concomitant Therapy
   a. Concurrent treatment with other investigational drugs.
   b. Concurrent treatment with any other anticancer therapy not specified in the protocol, including immunotherapy, hormonal therapy (adjuvant hormonal therapy is allowed), targeted therapy or biological therapies.
   c. Radiotherapy (except palliative intent) may not be given during the study. Palliative radiotherapy is permitted in cases where participant is deriving clinical benefit from study treatment.
   d. Prophylactic use of hematopoietic growth factors (e.g., G-CSF, GM-CSF, erythropoietin) during the DLT observation period in the Dose Escalation Part only.
H. Prior/concurrent clinical study experience
   a. Previous treatment with any other investigational agent for 21 days or 5 half-lives whichever is a shorter period to first IMP administration (Cycle 1, Day ≤1). AEs related to prior therapy, except for alopecia, must have resolved to ≤Grade.
I. Diagnostic assessments
   a. Total bilirubin >1.5×upper limit of normal (ULN), or >3×ULN for participants with known Gilbert's syndrome.
   b. Liver enzyme abnormalities, including >3×ULN for alanine aminotransferase, aspartate aminotransferase or >2.5×ULN alkaline phosphatase.
   c. Estimated creatinine clearance <30 mL/min (using the Cockcroft-Gault formula) OR estimated creatine clearance <30 mL/min/1.73 m² (for adolescents the Schwartz formula will be used).
   d. Grade 4 hematologic toxicity: bone marrow hypocellularity, anemia, thrombotic thrombocytopenic purpura.
   e. Grade ≥3 disseminated intravascular coagulation.
   f. White blood cell count ≥25 000/mm³. Use of hydroxyurea should be discontinued at least 1 day before first planned IMP administration.
J. Other Exclusions
   a. Individuals accommodated in an institution because of regulatory or legal order; prisoners or participants who are legally institutionalized.
   b. Pregnant and breast-feeding women, female participants of childbearing potential, and male participants with female partners of childbearing potential who are not willing to avoid pregnancy by using a highly effective method of contraception (2 barrier method or 1 barrier method with a spermicide, intrauterine device, or hormonal contraception with inhibition of ovulation, for 2 weeks prior to the first dose of CD123 NKCE, during treatment, and 12 weeks after the last dose of study treatment). A woman is considered of childbearing potential, i.e., fertile, following menarche and until becoming postmenopausal unless permanently sterile.
   c. Any clinically significant, uncontrolled medical conditions (including any serious systemic fungal, bacterial, viral, or other infection that is not controlled) that, in the Investigator's opinion, would expose excessive risk to the participant or may interfere with compliance or interpretation of the study results.
   d. History of solid organ transplant, including corneal transplant.
   e. Receipt of a live-virus vaccination within 28 days of planned treatment start. Seasonal flu vaccines or SARS-CoV-2 vaccines that do not contain live virus are permitted.
   f. Participant not suitable for participation, whatever the reason, as judged by the Investigator, including medical or clinical conditions, or participants potentially at risk of noncompliance to study procedures
   g. Participants are employees of the clinical study site or other individuals directly involved in the conduct of the study, or immediate family members of such individuals (in conjunction with section 1.61 of the ICH-GCP Ordinance E6).
   h. Any specific situation during study implementation/course that may rise ethics considerations.
   i. Sensitivity to any of the study interventions, or components thereof, or drug or other allergy that, in the opinion of the Investigator, contraindicates participation in the study.
   j. Average QTc (using the Fridericia correction calculation) >470 msec at screening.
   k. Pediatric Arm only: Participants with known inherited bone marrow failure syndromes (e.g., Bloom Syndrome, ataxia-telangiectasia, Fanconi anemia, Kostmann syndrome, Shwachman syndrome). Participants with Down syndrome with adequate organ function per discretion are allowed to participate in the study.
   l. French participants: Participant is under tutorship or curatorship; participant under safeguard of justice or deprived of his/her liberty by an administrative or court decision.
   m. Adult Expansion: Participants with MDS evolving from a pre-existing myeloproliferative neoplasm (MPN), MDS/MPN including chronic myelomonocytic leukemia (CMML), atypical chronic myeloid leukemia (aCML), unclassifiable MDS/MPN and therapy-related MDS (t-MDS).
   n. Confirmed diagnosis of acute promyelocytic leukemia (APL) and juvenile myelomonocytic leukemia (JMML) according to WHO 2022 classification.

### Study Intervention(s) and Concomitant Therapy

Study intervention is defined as any investigational intervention(s), marketed product(s), placebo, or medical device(s) intended to be administered to a study participant according to the study protocol.

Study interventions will be administered according to the following sequences: premedication for CD123 NKCE (30-60 min prior to CD123 NKCE administration), then CD123 NKCE.

Participants will receive study treatment until progression, occurrence of unacceptable toxicity, or other permanent discontinuation criteria, or completion of 13 cycles in the maintenance period. Beyond 13 cycles and outside of the frame of this clinical study, the Investigator may decide to pursue with the anti-leukemic treatment of his/her choice.

### Phase 1 Design (Dose Escalation)

During the adult arm dose escalation phase, the population to be treated includes adults and adolescents (≥12 years) with either relapsed or refractory AML, HR-MDS, or B-ALL that have no alternative treatment options. In the pediatric arm dose escalation, the population (1 to 17 years) to be treated includes relapsed or refractory AML, B-ALL, or BPDCN that have no alternative treatment options. The adult and pediatric escalation part aims to determine the MTD or MAD and preliminary RDEs of the CD123 NKCE based on the occurrence of DLT in Cycle 1. At the end of dose escalation, preliminary RDEs will be identified. Separate RDEs may be identified for the adult and pediatric populations.

There is no formal sample size calculation in the adult dose escalation phase. CD123 NKCE will be administered to participants with r/r AML, HR-MDS, B-ALL, or BPDCN. It is anticipated that approximately 66 participants will be enrolled in the adult dose escalation part with expected assessment of about 11 main DLs while intermediate DLs may be investigated in case of dose or schedule modifications. Approximately 24 participants will be enrolled in the dose escalation part for pediatric with expected assessment of about 3 main DLs while intermediate DLs may be investigated in case of dose or schedule modifications. The actual sample size will vary depending on DLTs observed and number of DLs actually explored and the number of participants enrolled by not DLT-evaluable.

### Rationale for Starting Dose and Dose Escalation Scheme

### Starting Dose for the Adult Arm

The proposed IV starting dose of 10 μg/kg for the adult arm is based on mechanistic PK/PD model and considers the estimated exposure in human (Cₘₐₓ AUC_{0-3d}) equivalent to 1/10 (safety factor) the exposure quantified in non-human primate (NHP) at the No Observed Adverse Effect Level (NOAEL) defined at 3000 μg/kg/adm after 5-weeks twice-weekly dosing and/or SC dosing at the highest dose tested (3 mg/kg/adm).

As recommended by the International Consortium for Innovation and Quality in Pharmaceutical Development (IQ) Assessment, PK/Pdy modeling is the preferred approach for clinical starting dose selection as it integrates the mechanism of action and the target dynamics. Moreover, all in vitro and in vivo nonclinical data helped to anticipate any potential safety risks, ie:
In in vitro human PBMC assay, CD123 NKCE exhibited minimal cytokine release at concentrations up to the highest concentration tested of 10 μg/mL, corresponding to about 1785-fold higher than CD123 NKCE EC50 (ie, 5.60 ng/mL) for cytotoxicity against basophils.
In in vitro human whole blood assay, CD123 NKCE concentration from 0.25 μg/mL (2.8 fold above estimated human Ceoi at 10 μg/kg, 0.09 μg/mL) induced mild cytokine release, mainly IL-6 and IFN-γ mean 13-21-fold increase in IL-6 and mean 10-24-fold increase in IFN-γ. However, it should be noted that no further cytokine induction was observed at higher concentrations tested up to 100 μg/mL, indicating a lack of dose dependency of the effect. This contrasts with much higher cytokine release induced by the CD3×CD123 T-Cell Engager (TCE) positive control at 10 μg/mL (mean 4390-fold in IL-2, mean 1697-fold in IFN-γ, mean 1087-fold in TNF-α and mean 65-fold in IL-6).
In NHP GLP repeated dose toxicity study, the safety profile was found very favorable at doses that induced pharmacodynamic effects consisting in a CD123 NKCE-related decrease in total CD123+ cells in both bone marrow and blood, as well as decrease in NK cells expressing NKp46 in blood. Indeed, at the highest dose tested of 3000 μg/kg/adm (NOAEL) administered twice-weekly for 5 weeks, very transient and non-adverse increase in some cytokines (IL-1Ra, IL-10, MIP-1β and TNFα) after the last (9^{th}) IV infusion and no clinical signs, temperature increase or changes in ECG, blood pressure, respiratory rate, or neuro-behavior parameters were noticed. Moreover, there were no CD123 NKCE-related microscopic findings observed in any organ.
Based on nonclinical findings and PK/Pdy modeling, the estimated human exposure at 10 μg/kg, the proposed starting dose, is 2-log lower than the exposure observed in NHP at NOAEL.

Intra-participant dose escalation is implemented in the adult arm IV escalation part to limit the number of participants treated at sub-therapeutic doses. This is particularly important in the initial DLs. The dose for subsequent induction cycles and maintenance cycles will utilize the full treatment dose reached at the end of Induction Cycle 1.

Dose escalation is planned through approximately 11 main dose levels (DLs). Intra-participant dose escalation and twice-weekly dosing will allow to avoid sub-therapeutic exposure at the lower DLs. The approach is supported by PK/PD modeling where from DL1/DL2, the model would predict at least 95% depletion of CD123+ blasts to be reached by the end of Week 1. Maximal dose increments between DLs for both priming doses and full dose will not exceed half a log (300%). A DL(−1) or intermediate DLs may be tested if unacceptable or exceeding toxicity is observed. The proposed dose levels and dose escalation scheme is depicted in Tables 13 and 14.

TABLE 13
Dose escalation: Induction Cycle 1 (all doses in μg/kg)

| Dose | Maximal CD123 NKCE dose (μg/kg) | | | | | |
|---|---|---|---|---|---|---|
| Level | Week 1 | | Week 2 | | Week 3 | Week 4 |
| (DL) | Day 1 | Day 4 | Day 8 | Day 11 | Day 15 | Day 22 |
| DL(−1)** | 3 | 10 | 30 | 30 | 30 | 30 |
| DL1 | 10 | 30 | 100 | 100 | 100 | 100 |
| DL1bis*** | 30 | 100 | 100 | 100 | 100 | 100 |
| DL2A* | 13 | 40 | 130 | 130 | 130 | 130 |
| DL2B* | 15 | 50 | 150 | 150 | 150 | 150 |
| DL2C* | 20 | 60 | 200 | 200 | 200 | 200 |
| DL2 | 30 ^{#} | 100 | 300 | 300 | 300 | 300 |
| DL3A* | 40 | 120 | 400 | 400 | 400 | 400 |
| DL3B* | 45 | 150 | 450 | 450 | 450 | 450 |
| DL3C* | 60 | 200 | 600 | 600 | 600 | 600 |
| DL3 | 100 | 300 | 1000 | — | 1000 | 1000 |
| DL4 | 300 | — | 1000 | — | 1000 | 1000 |
| DL5A* | 400 | — | 1300 | — | 1300 | 1300 |
| DL5B* | 450 | — | 1500 | — | 1500 | 1500 |
| DL5C* | 600 | — | 2000 | — | 2000 | 2000 |
| DL5 | 1000 | — | 3000 | — | 3000 | 3000 |
| DL6A* | 1300 | — | 3000 | — | 3000 | 3000 |
| DL6B* | 1500 | — | 3000 | — | 3000 | 3000 |
| DL6C* | 2000 | — | 3000 | — | 3000 | 3000 |
| DL6 | 3000 | — | 3000 | — | 3000 | 3000 |
| DL7A | 4000 | — | 4000 | — | 4000 | 4000 |
| DL7B | 4500 | — | 4500 | — | 4500 | 4500 |
| DL7 | 6000 | — | 6000 | — | 6000 | 6000 |
| DL 1 mg | 1000 | — | 1000 | — | 1000 | 1000 |
| DL 0.6 mg | 600 | — | 600 | — | 600 | 600 |
| DL 0.75 mg | 750 | — | 750 | — | 750 | 750 |
| DL 1.5 mg | 1500 | — | 1500 | — | 1500 | 1500 |
| DL 2 mg | 2000 | — | 2000 | — | 2000 | 2000 |

*Optional intermediate dose levels with increments calculated when compared to the DL just previously tested. These intermediate DLs could be tested as per Study Board decision based on available data. Other DLs that may include more intensive priming as well as other schedules could be tested also based on available data as per Study Board decision.
**DL(−1) may be tested if unacceptable or exceeding toxicity is observed at DL1.
***Intermediate dose level that may be explored if the 100 mg/vial DP is not available upon completion of DL1. Upon completion of DL1bis dose, escalation will continue.
^{#} If DL1bis is tested, cleared and BLRM recommends to escalate to the main DL2, a lead-in dose of 100 μg/kg may be substituted for the dose of 30 μg/kg on Day 1 at DL2.
§Doses indicated are the maximum dose that could be tested on a given day at a given DL.

TABLE 14
Dose escalation: induction Cycle 2 and beyond (all doses in μg/kg)

| Dose | Maximal CD123 NKCE dose (μg/kg) | | | | | |
|---|---|---|---|---|---|---|
| Level | Week 1 | | Week 2 | | Week 3 | Week 4 |
| (DL) | Day 1 | Day 4 | Day 8 | Day 11 | Day 15 | Day 22 |
| DL(−1)** | 30 | — | 30 | — | 30 | 30 |
| DL1 | 100 | — | 100 | — | 100 | 100 |
| DL1bis*** | 100 | — | 100 | — | 100 | 100 |
| DL2A* | 130 | — | 130 | — | 130 | 130 |
| DL2B* | 150 | — | 150 | — | 150 | 150 |
| DL2C* | 200 | — | 200 | — | 200 | 200 |
| DL2 | 300 | — | 300 | — | 300 | 300 |
| DL3A* | 400 | — | 400 | — | 400 | 400 |
| DL3B* | 450 | — | 450 | — | 450 | 450 |
| DL3C* | 600 | — | 600 | — | 600 | 600 |
| DL3 | 1000 | — | 1000 | — | 1000 | 1000 |
| DL4 | 1000 | — | 1000 | — | 1000 | 1000 |
| DL5A* | 1300 | — | 1300 | — | 1300 | 1300 |
| DL5B* | 1500 | — | 1500 | — | 1500 | 1500 |
| DL5C* | 2000 | — | 2000 | — | 2000 | 2000 |
| DL5 | 3000 | — | 3000 | — | 3000 | 3000 |
| DL6A* | 1300 | — | 3000 | — | 3000 | 3000 |
| DL6B* | 1500 | — | 3000 | — | 3000 | 3000 |
| DL6C* | 2000 | — | 3000 | — | 3000 | 3000 |
| DL6 | 3000 | — | 3000 | — | 3000 | 3000 |
| DL7A | 4000 | — | 4000 | — | 4000 | 4000 |
| DL7B | 4500 | — | 4500 | — | 4500 | 4500 |
| DL7 | 6000 | — | 6000 | — | 6000 | 6000 |
| DL 1 mg | 1000 | — | 1000 | — | 1000 | 1000 |
| DL 0.6 mg | 600 | — | 600 | — | 600 | 600 |
| DL 0.75 mg | 750 | — | 750 | — | 750 | 750 |
| DL 1.5 mg | 1500 | — | 1500 | — | 1500 | 1500 |
| DL 2 mg | 2000 | — | 2000 | — | 2000 | 2000 |

*Optional intermediate dose levels with increments calculated when compared to the DL just previously tested. These intermediate DLs could be tested as per Study Board decision based on available data. Other DLs that may include more intensive priming as well as other schedules could be tested also based on available data as per Study Board decision.
**DL(−1) may be tested if unacceptable or exceeding toxicity is observed at DL1.
***Only if 100 mg/vial DP was not available upon completion of DL1.
§Doses indicated are the maximum dose that could be tested in a given DL according to the maximum dose administered in Cycle 1.

### Starting Dose for the Pediatric Arm

The pediatric arm IV starting dose is 1000 μg/kg and is selected based on the adult escalation data. Preliminary clinical safety data showing no dose limiting toxicities were observed in 1000 μg/kg and other dose levels tested in adults. 1000 μg/kg also demonstrated preliminary clinical activity in adults, supporting the starting dose in pediatric arm participants. Pediatric arm participants aged 1 to 11 will be treated after the respective dose level in the adult arm has been declared safe by the Study Board. In the pediatric arm IV dose-escalation there is no LID and the maximum increase between dose levels is approximately a half-log (300%). The maximum dose increase will be modified for subsequent dose levels for TEAEs Grade ≥2 or DLTs.

The pediatric arm dose escalation part is planned for approximately 3 main dose levels (Table 15).

TABLE 15
Pediatric Dose escalation: induction and maintenance (all doses in μg/kg)

| Dose Level | Induction | Maintenance |
|---|---|---|
| (DL) | Days 1, 8, 15, 22 | Days 1, 29 |
| DL(−1)* | 600 | 600 |
| DL1 | 1000 | 1000 |
| DL1a** | 1500 | 1500 |
| DL1b** | 2000 | 2000 |
| DL2** | 3000 | 3000 |

*DL(−1) may be tested if unacceptable or exceeding toxicity is observed at DL1
**Optional intermediate dose levels with increments calculated when compared to the DL just previously tested. These intermediate DLs could be tested as per Study Board decision based on available data.

### Recommendation for Dose Escalation (Adults and Pediatrics)

A DL starts with an initial lead-in dose (LID), followed by intra-participant stepwise escalation until the full target dose is reached. LID may constitute a priming/desensitizing approach to mitigate the occurrence of CRS, however, since CRS is not anticipated for CD123 NKCE, the LID is gradually phased out during the course of the dose escalation.
In the pediatric arm participants aged 1 to 11 will be treated after the respective dose level in the adult arm has been declared safe by the Study Board.

The maximum dose increment between dose levels for the lead-in dose is approximately a half-log (300%) and the maximum dose increment between full dose levels is approximately a half-log (300%). The maximum dose increment for the LID and full dose will be reduced for subsequent dosing cohorts if a participant in a given cohort experiences a Grade ≥2 TEAE or DLTs. Within the main dose levels a maximum of two approximate half-log (300%) stepwise intra-participant dose increases occur between the LID to the full dose resulting in an approximate 1 log increase.

Continued stepwise dose escalation for a participant should be stopped for a Grade 2 event. Upon resolution, treatment should resume at the dose at which the Grade 2 event occurred.

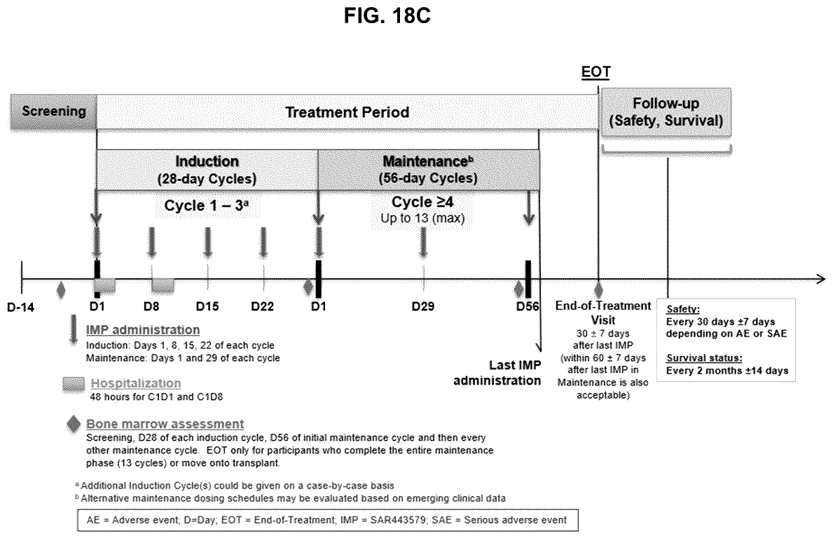

Decisions to escalate will take into account the results of clinical safety. Consecutive cohorts of participants will be treated at CD123 NKCE dose levels (DLs) per decisions of the Study Board. The DLT observation period is 28 days. Depending on the dose level, CD123 NKCE will be administered IV twice-weekly or once-weekly in the first 1 or 2 weeks of Cycle 1 on Days 1, 4, 8 and 11 and once-weekly during the last 2 weeks of Cycle 1 (Day 15 and Day 22) and all subsequent induction cycles (Days 1, 8, 15, and 22). When supported by PK data, alternative schedules may be tested (FIGS. 18A and B). A participant in a cohort will be considered evaluable for DLT assessment if he/she receives at least 75% of his/her planned CD123 NKCE doses during the final 28 days of the treatment (i.e., DLT period) and is evaluated for 28 days, or if a DLT occurs during the DLT period. Participants not evaluable for DLT assessment in the dose escalation phase (eg, early progressive disease before the end of the DLT period; any missing DLT assessment parameters) will be replaced if the minimum number of DLT-evaluable participants in the current cohort is not met.

Unless explicitly indicated for alternative dosing, DLs refer to the 11 primary dose levels for adults or up to 3 primary dose levels for pediatrics, which are evaluated in a sequential manner using the Bayesian Logistic Regression Model (BLRM). The BLRM method will be used to provide dose recommendations on dose escalation for both adults and pediatrics. This adaptive dose escalation is based on a statistical (2-parameter logistic) model for the probability of DLT during the DLT period as a function of CD123 NKCE dose. The model will be used to estimate whether the probability of DLTs at each candidate DL during a DLT period is within a targeted toxicity interval of 15% to 25% and has a less than 0.40 probability of a true DLT rate greater than the target range.

At each dose decision, the model will be used to estimate the following for each DL within the planned dose range: (a) the probability of a DLT rate within the target range, and (b) the probability of a DLT rate above the target range. The “best” MTD estimate from the model at any point is the DL with the highest probability of (a) and with less than 0.40 probability of (b). With the following exceptions, the design's recommended dose for the next cohort of participants will be the model's best MTD estimate, which may be lower, higher, or the same as the current DL:
Enrollment at the next DL may not proceed before at least 3 participants treated at the current DL have been followed for at least 28 days (DLT period) and are evaluable for DLT assessments with no DLT reported.
If 1 of the first 3 participants experiences a DLT at any given DL (except DL1), the BLRM will be updated and if the recommendation is a continuation at the same DL, then 3 additional participants will be enrolled in that DL cohort.
Once the DLT status for an additional participant (that may be enrolled at the same DL after the third participant due to rapid enrollment) is available and if this participant experiences a DLT after the dose escalation, decision is made to proceed with a higher DL based on the first 3 participants (who have completed DLT period). The Sponsor will update the Bayesian model based on all participants at this DL to determine whether continuation of treatment at this new (higher) DL is acceptable, or if more participants should be evaluated at the previous DL (ie, the DL the first 3 participants were tested).

Dose escalation will be indicated by the model if the probability of DLT within the targeted toxicity interval at the next level is greater than at the current level. Dose de-escalation will be indicated if the probability of DLT within the targeted toxicity interval at a lower level is greater than at the current level. Otherwise, subsequent participants will be treated at the current DL.

Adolescents (12 to 17 years old) should be prioritized for enrollment into the pediatric portion of the study. If no pediatric slots are available, the Study Board may consider if the benefit/risk is acceptable on a case by case basis and enroll adolescents into the adult expansion. Participants that turn 18 during the course of the study would remain within their initially assigned arm
A minimum of 3 DLT-evaluable participants will be treated at each DL for the escalation part. De-escalation to DL(−1) occurs if any participant in DL1 experiences a Grade ≥3 CRS or neurotoxicity. Recommendations for the dose level at which the next cohort of participants may be treated will be provided by the BLRM. However, the final decision whether to escalate to the next DL, de-escalate or stay at the current DL, or to evaluate alternative lower dose level will be determined by the Study Board based on evaluation of available safety data (including DLTs, adverse events [AEs] and serious adverse events [SAEs]), clinical PK and PDy. If an alternative DL is chosen, a minimum of 3 participants will be enrolled in the chosen DL. There will be at least 7 days between dosing of the first participant and all subsequent participants in each dose level. Adolescent participants (aged 12 to 17 years) may be enrolled in the adult arm IV escalation part when the first adult treated at DL3 has completed Cycle 1 without occurrence of DLT and provided that PK data support pharmacologically relevant exposure.
The actual dose selected at each dose decision and the MTD identified at the conclusion of the dose escalation part will be determined by the Study Board, which may select a dose different than the model's recommendation (which may include, but not limited to, a partial increase or decrease). The Study Board may discuss dose modifications and dose expansion on a case-by-case basis.
In case the MTD is not reached, the recommended dose(s) for expansion part will be decided by the Study Board, based on DLT (if established) and other relevant data such as safety, anti-tumor activity, PK, and Pdy information. Decisions regarding participant treatment and dose expansion will be clearly documented in the meeting minutes of the Study Board.
Dose escalation will be stopped as soon as the MTD is determined. If a MTD is not determined, dose escalation will continue until the MAD is achieved.

Alternative dose levels for dose escalation: The following rules will apply in the specific situations during dose titration:

TABLE 16

| | Impact on |
|---|---|
| IMP-related AEs observed at Cycle 1 | Dose Escalation Process |
| Grade ≥ 3 CRS or neurotoxicity in any | De-escalate to DL minus 1 |
| participant of DL1 cohort | |
| One Grade ≥ 2 AE in any participant of | Limit the maximum dose |
| a given cohort | increment to 100%* |
| Two or more Grade ≥ 2 AEs or one DLT | Limit the maximum dose |
| in any participant in a given cohort | increment to 50%* |
| Two DLTs in any participant of a given | Limit the maximum dose |
| cohort | increment to 30%* |

*Limitation in dose increment including both priming doses and full treatment doses for the subsequent cohorts. If the maximum dose increment for escalation is modified due to the occurrence of a Grade ≥2 AE or DLT then future dose escalation decisions will proceed by the revised dose increment. (For example: A Grade ≥2 AE during the DLT period at DLX that does not resolve to Grade 1 leads to a maximum of 100% increment for DLX + 1 and all subsequent dose levels, DLX + 2, etc). AE is referring to treatment emergent adverse events (TEAE).

Grade 2 AEs, whether related or not to the IMP in absence of clear evidence to the contrary after validation by the Study Board, and if not related to disease progression, will lead to subsequent modification in dose escalation strategy, except for the following:
Alopecia
Grade 2 fatigue, asthenia, fever, anorexia, or constipation
Grade 2 nausea, vomiting or diarrhea if successfully managed with optimal medical support
Grade 2 isolated electrolyte abnormalities (i.e., those occurring without clinical consequences)
Grade 2 laboratory abnormalities that are considered not to be clinically relevant (e.g., Grad 2 GGT elevation)
Infection, bleeding, or other expected direct complication of cytopenias due to active underlying leukemia
Grade 2 IRR, if successfully managed and which resolves within 24 hours
   The Study board, upon review of the safety, PK and PD data, may recommend an intermediate dose level and schedule other than what is previously defined. Any intermediate dose level selected will observe at maximum an approximate half-log increase from the preceding LID and full dose levels and remain consistent with the rules as defined. Decision to explore an intermediate dose level will be clearly documented in the minutes of the Study Board meetings.

### Recommended Dose(s) for Expansion (RDE)

The RDE, which is also referred to as the recommended Phase 2 dose (RP2D), will be determined by the Study Board based at the end of the dose escalation of both the adult and pediatric arms on the totality of the information available at all dose levels (preliminary efficacy safety data including incidence of AE, SAE and DLTs, tolerability; PK, PD, and immunophenotypic changes will be considered, but also evidence of antitumor response may be taken into account) and should not exceed the MTD.

### Adult Arm

Upon completion of the Adult arm Dose Escalation and based on the analysis of the totality of the available data the Study Board will determine the preliminary RDEs for dose expansion/optimization. Based on the totality of the data generated (preliminary clinical safety, efficacy, PK, biomarker and non-clinical data) during the adult arm dose expansion 3 dose levels were selected as preliminary RDE for further evaluation: 0.6 mg/kg (600 μg/kg), 1 mg/kg (1000 μg/kg) and 1.5 mg/kg (1500 μg/kg).

### Pediatric Arm

In the pediatric arm expansion/optimization part, the preliminary RDE(s) will be selected by the Study Board based on the totality of the data generated from the adult and pediatric arm.

The preliminary RDEs selected for the pediatric arm may be different from the adult preliminary RDEs.

### Cohorts for the Expansion/Optimization Phase:

Cohort A, the safety and preliminary efficacy of one of the preliminary RDEs (e.g., 1 mg/kg) may be confirmed through recruitment of up to 20 participants prior to initiation of further expansion/optimization. The anti-tumor activity of CD123 NKCE may be subsequently evaluated in up to 60 additional participants (20 per dose level) with R/R AML which may be randomized between 2 and up to 3 different dose levels selected from 0.6 mg/kg, 1 mg/kg and 1.5 mg/kg, followed by potential expansion of one dose level with an additional 20 participants to further examine anti-leukemic activity. Note: Participants enrolled in the pRDE cohort (e.g., 1 mg/kg) may be considered towards the sample population of the same dose level to be randomized
Cohort B: The safety and preliminary efficacy of one of the preliminary RDEs (e.g., 1 mg/kg) may be confirmed through recruitment of up to 10 participants prior to initiation of further expansion/optimization, then up to 40 participants with MDS may be randomized 1:1 at selected RDEs. Following interim analysis one of the dose levels may be expanded by up to an additional 20 participants to confirm the anti-leukemic activity. Note: Participants enrolled in the pRDE cohort (e.g., 1 mg/kg) may be considered towards the sample population of the same dose level to be randomized
Cohort C (adult arm; ≥18 years old): Japan specific: Up to 9 participants with R/R AML, HR-MDS, and B-ALL will be enrolled at 1 mg/kg.
Cohort D (pediatric arm; 1 to 17 years old): The preliminary RDEs identified based on the totality of data from the pediatric and adult escalation, may undergo 1:1:1 (up to 3 dose levels) randomization with up to 20 R/R AML participants per dose level (total up to 60 participants). Following interim analysis one of the dose levels may be expanded by up to an additional 20 participants to further examine anti-leukemic activity.

Definition of DLT: DLT is defined as the occurrence, within the first 28-day cycle of any of the following using National Cancer Institute Common Terminology Criteria for adverse Events (NCI CTCAE) version 5.0 or ASTCT criteria, whether related or not to the study treatment in the absence of clear evidence to the contrary, and if not related to disease progression:

TABLE 17
Dose-Limiting toxicities
Hematologic Abnormalities

| | |
|---|---|
| Grade 4 (i.e., new onset or worsening of hematologic | Grade 4 bone marrow hypocellularity, if not caused |
| abnormalities after administration of CD123 NKCE, | by disease progression and not improved to baseline |
| in absence of underlying leukemia) | value or Grade ≤ 1 within 14 days |
| | Grade 4 decreased neutrophils last >14 days in the |
| | absence of underlying disease |
| | Grade 4 febrile neutropenia |
| | Grade 4 decreased platelet count lasting >14 days in |
| | the absence of underlying disease |
| | Grade 4 anemia, if not improved to baseline value or |
| | Grade ≤ 1 within 14 days |
| Grade 3 | Thrombocytopenia with bleeding requiring |
| | transfusion |

Non-Hematologic Abnormalities

| | |
|---|---|
| Any Grade ≥3 toxicities excluding: | Alopecia |
| | Grade 3 fatigue, asthenia, fever, anorexia, or |
| | constipation |
| | Grade 3 nausea, vomiting or diarrhea not requiring |
| | tube feeding, total parenteral nutrition, or requiring or |
| | prolonging hospitalization |
| | Infection, bleeding, or other expected direct |
| | complication of cytopenias due to active underlying |
| | leukemia |
| | Grade 3 infusion related reaction including CRS, if |
| | successfully managed and which resolves within 72 |
| | hours |
| | Grade 3 or 4 tumor lysis syndrome (TLS) if it is |
| | successfully managed clinically and resolves within 7 |
| | days without end-organ damage |
| | Grade 3 or 4 isolated electrolyte abnormalities (those |
| | occurring without clinical consequence) that resolve, |
| | with or without intervention, to <Grade 2 levels in |
| | <72 hours |
| | Grade 3 laboratory abnormalities that resolve to |
| | ≤Grade 2 levels within 72 hours |

Any toxicity related to CD123 NKCE resulting in a treatment delay of >2 weeks due to delayed recovery to
baseline or Grade ≤ 1 or resulting in dose reduction.

### Dose Expansion Optimization Phase

Continuous safety and efficacy monitoring will be performed using Bayesian methods at interim analyses. The sample size for the dose expansion phase is not based upon a power calculation but is intended to provide preliminary information on the activity of CD123 NKCE as a single agent.

The first portion of the dose expansion/optimization (Cohorts A and B) will enroll up to 10-20 participants tested at one of the selected RDEs (e.g., 1 mg/kg) to further evaluate safety and preliminary efficacy.

As a general rule, continuous safety and efficacy monitoring will be performed for all cohorts using Bayesian methods during the conduct of the expansion/optimization and could trigger early decisions to stop or expand a specific cohorts/dose levels.

For Cohort A, following the completion of the preliminary RDE assessment in up to 20 participants, a total of up to 60 participants will be randomized across at least 2 and up to 3 different dose levels (20 participants per dose). Note: Participants enrolled in the pRDE cohort (eg, 1 mg/kg [1000 jig/kg]) may be considered towards the sample population of the same dose

level to be randomized. Upon completion or early termination of Cohorts A1, A2, and/or A3 the enrollment may be paused for interim analysis. After interim analysis, decision about re-opening an additional 20 participants at a select RDE will be made.

For Cohort B, following the completion of the pRDE assessment in up to 10 participants in MDS, a total up to 40 participants will be randomized in a 1:1 ratio (up to 20 per dose level). Participants enrolled in the pRDE (eg, 1 mg/kg [1000 pig/kg]) cohort may be considered towards the sample population of the same dose level to be randomized. Upon completion of Cohorts B1 and/or B2 or early termination of a dose level then enrollment may be temporarily paused for interim

analysis. Following the interim analysis, a decision will be made regarding the possibility of reopening enrollment for an additional 20 participants at a specified RDE. Cohort C will enroll up to 9 participants and will evaluate preliminary safety and PK in the Japanese population.

NOTE: For participants in the expansion Cohort A and Cohort B randomized to dose levels [e.g. 0.6 mg/kg (600 μg/kg), 1.5 mg/kg (1500 μg/kg)] other than the dosage tested in the pRDE cohort [eg, 1 mg/kg (1000 μg/kg)], may be cross treated at pRDE dose (if declared efficacious by the study board), in case no clinical response is observed after 3 induction cycles.

In Cohort D, the expansion/optimization part for the pediatric arm up to 60 pediatric arm

participants with R/R AML (Cohort D) may be randomized across at least 2 and up to 3 different dose levels (20 participants per dose). Upon completion or early termination of Cohorts D1, D2, and/or D3 the enrollment may be paused for interim analysis. After interim analysis, decision about re-opening an additional 20 participants at a select RDE will be made.

### Potential Adverse Reactions

Potential risks listed below in Table 18 are based on safety information from ex vivo experiments on human PBMCs, toxicology studies in NHP with CD123 NKCE, class effects of biologicals, and safety information from CD123 immune cell engagers, such as T-cell engaging antibodies or CAR-T cell therapy.

TABLE 18
Potential Adverse Reactions with CD123 NKCE

| Adverse Reaction | Description |
|---|---|
| Infusion-related reactions (IRR) | As a biological entity, CD123 NKCE may induce hypersensitivity |
| | reactions of various degree of severity. Administration of |
| | talacotuzumab, an investigational humanized, Fc competent anti- |
| | CD123 monoclonal antibody, has been associated with IRR in Phase 2 |
| | and Phase 3 clinical trials when administered in combination with |
| | decitabine. Participants routinely receive premedication as detailed in |
| | the protocol prior to CD123 NKCE administration, to prevent or |
| | reduce the incidence or severity of IRRs. Recommendations for |
| | intervention in case of IRR are described in the protocol. |
| Tumor lysis syndrome (TLS) | Rapid destruction of malignant cells in patients with hematologic |
| | malignancies has been associated with TLS. Patients with a high |
| | amount of blast cells are at higher risk for experiencing TLS. |
| Myelosuppression | No hematological findings and no bone marrow suppression were |
| | observed in monkeys up to 3 mg/kg/adm when given twice weekly for |
| | 5 weeks suggesting a low risk for myelosuppression with CD123 |
| | NKCE. Cytopenias (including anemia, decreased platelets, and |
| | lymphocytes and leukocytes) have been reported in clinical trials of |
| | patients receiving T-cell engaging antibodies (e.g., CD123 targeting |
| | TCE flotetuzumab). |
| Cytokine Release Syndrome (CRS) | In the exploratory toxicity study in cynomolgus monkeys (100 and |
| | 3000 μg/kg/adm, once a week for 4 weeks by 1-hour IV infusion), |
| | very low IL-2, IL-6, and IL-10 cytokine release was observed afer |
| | each dosing in all treated animals whatever the dose. IN the pivotal |
| | toxicity study (100 and 3000 μg/kg/adm by 1 hour IV infusion, and |
| | 3000 μg/kg/adm by SC, twice a week for 5 weeks), there was very |
| | minimal to moderate increases of some cytokines (IL-1Ra, IL-10, |
| | MIP1β, and TNFα) after the fifth and the last ninth IV infusion at 3000 |
| | μg/kg/adm. Considering the magnitude of changes and that the levels |
| | returned to baseline after 5 or 24 hours these changes were considered |
| | non-adverse. In human studies, T cell engaging therapeutics (e.g., |
| | CD123 targeting TCE flotetuzumab and CAR-T cells) have frequently |
| | been associated with episodes of CRS that can be severe. NK cell |
| | engaging therapeutics do not appear to induce signficant cytokine |
| | release manifestations and it is anticipated the risk for CRS would be |
| | much lower than T-cell engaging therapy. Preclinical studies with |
| | CD123 NKCE suggest a mnimal risk for CRS following |
| | administration, and thus CRS remains a potential risk. |
| Immune Effector Cell-Associated | In clinical studies with flotetuzumab, a TCE that also induces CRS, 2 |
| Neurotoxicity Syndrome (ICANS) | central neurological DLTs were reported in their Phase 1 study |
| | (NCT04681105) (Grade 3 delirium and Grade 3 acute confusion). |
| | Immune effector cell-associated neurotoxicity syndrome (ICANS), |
| | although not always, is often associated with cytokine release. No |
| | neurotoxicity was observed in monkeys up to 3 mg/kg/adm when |
| | given twice weekly for 5 weeks. As neurotoxicity has not been |
| | observed and as cytokine release is minimal with CD123 NKCE in |
| | nonclinical studies, it is considered that there is a low risk of ICANS, |
| | although it cannot be excluded. |
| Immunogenicity | CD123 NKCE may have immunogenic potential and induce ADA that |
| | may impact its pharmacological properties, induce the formation of |
| | immune complexes, and may trigger AEs. While a majority of |
| | biologic therapeutics elicit some titer of ADA, a majority of them do |
| | not cause clinical side effects. |

### General Precautions

Participants should be monitored during the infusion and should routinely receive premedication prior to CD123 NKCE administration, to prevent or reduce the incidence or severity of IRRs. Premedication includes acetaminophen 650 to 1000 mg orally (or equivalent), and diphenhydramine 25 to 50 mg IV (or equivalent), 30 to 60 minutes prior to administration of CD123 NKCE in Cycle 1 and is optional in the following cycles if no IRR occurred in the first cycle.

If IRR >Grade 2 occurs in a trial participant, the participant's CD123 NKCE infusion will be interrupted for up to 4 h. The participant will be treated with antihistamine (eg, diphenhydramine or loratadine), acetaminophen, and dexamethasone based on investigator discretion. Once IRR has

resolved to ≤Grade 1, the infusion will be restarted at 50% of the initial infusion rate*.
If IRR Grade 2 persists for >4 h or recurs after restart of infusion, the participant's CD123 NKCE infusion will not be resumed on that day of infusion and the participant should be premedicated for the next scheduled dose. The next infusion should be given at half the initial infusion rate* until no re-occurrence of IRR ≥Grade 2, whereafter subsequent infusions can be administered at the initial infusion rate*

In case of Grade 3 IRR, resumption of treatment following resolution ≤Grade 1 may be considered following a discussion between the investigator and the Sponsor. CD123 NKCE should be permanently discontinued for Grade 4.

*Initial infusion rate is defined as the planned infusion rate prior to the first occurrence of IRR of study participant. Participants should be monitored for TLS. During Cycle 1, blood chemistry (creatinine, potassium, corrected calcium, phosphorus, and uric acid) will be monitored every 8 hours (+30 min) for 72 hours after start of infusion to monitor TLS following CD123 NKCE dosing on Day 1, Day 4, and Day 8 of twice weekly dosing or D1 and D8 (IV dosing) of once-weekly dosing of CD123 NKCE during Induction Cycle 1. If TLS occurs, appropriate treatment will be initiated that will include correction of serum corrected calcium, potassium, or phosphorus abnormalities, and treatment of hyperuricemia with allopurinol and/or rasburicase. Good hydration should be maintained at all times.

Participants should be monitored for signs and symptoms of infection and signs or symptoms of bleeding as a possible consequence of myelosuppression (neutropenia and thrombocytopenia). Potential infections have to be investigated with cultures and/or radiologic evaluation or other tests, as needed, to determine the etiology. Infections will be managed with appropriate antimicrobial therapies and supportive care. For bleeding disorders, platelet transfusions will have to be performed when indicated.

Participants need to be closely monitored for signs and symptoms of CRS during the first 12 days of Cycle 1. Fever can frequently occur with immune cell enhancers and may possibly evolve into flu-like symptoms or could be an early manifestation of CRS. Fever or flu-like symptoms should be managed according to institutional standards. CRS should be graded and managed as per the American Society for Transplantation and Cellular Therapy (ASTCT) guidelines included in the protocol. If a participant develops CRS ≥Grade 2, sites are highly encouraged to draw an additional blood sample for cytokines levels, and C-reactive protein and ferritin (by local laboratory) prior to the administration of tocilizumab.

Sites should have tocilizumab or other institutional recommended interventions available along with access to an intensive care unit (ICU), in case participants develop CRS. Participants at risk for CRS should be advised not to drive a car, operate heavy machinery, or do other dangerous activities while on treatment.
Acetaminophen 650 mg to 1000 mg orally (or adult/pediatric equivalent), optionally thereafter, and diphenhydramine 25 to 50 mg IV (or adult/pediatric equivalent), optionally thereafter, will be administered prophylactically 30 to 60 minutes prior to CD123 administration during Cycle 1.
If CRS requiring mild intervention occurs, as indicated by clinical symptoms, appropriate symptomatic treatment may include, but is not limited to administration of acetaminophen, oxygen, and IV fluids.
For participants with comorbidities, older age, oxygen requirement, hypotension, or participants in whom symptoms (e.g., high grade fever) do not respond to antipyretics within 72 hours, treatment with corticosteroids and/or tocilizumab should be considered, as per guidance for Grade 3 events.
If CRS requiring moderate to aggressive intervention occurs, high dose or multiple vasopressors, oxygen, mechanical ventilation, and/or other supportive care as needed should be administered.
In moderate or severe cases dexamethasone 10 mg IV should be given every 6 hours with maximum of 8 doses or until resolution of CRS to ≤Grade 1. If necessary, the dose may be increased to 20 mg IV every 6 hours.
The recommended dose of tocilizumab for treatment of CRS, when indicated for more severe cases or lack of improvement on dexamethasone, is 8 mg/kg IV. Doses of tocilizumab should not exceed 800 mg per infusion. More detailed guidance is provided in the protocol and the site may also follow local standard practices for management of CRS. Prophylactic administration of tocilizumab prior to the next dose is not allowed.

Immune Effector Cell-Associated Neurotoxicity syndrome (ICANS) is a neuropsychiatric syndrome which is frequently associated with CRS; however, it is specifically excluded from the definition of CRS and can occur during the course of CRS, after its resolution, or independently from CRS. Clinical findings can be progressive and may include aphasia, altered level of consciousness, impairment of cognitive skills, motor weakness, seizure, and cerebral edema. Severity is evaluated using the ASTCT Consensus grading scale, with ICE (Impact+Confidence+Ease) score for encephalopathy assessment. Recommendations for ICANS management mainly include the use of steroids, whereas tocilizumab should only be used in the context of CRS, as outlined in the protocol. The proposed management should be considered only as recommendations and in light of recommendations from site specialist. The site may use local standard operating procedure (SOP) for management.

### Efficacy Assessments

In the Escalation Phase, efficacy assessment will be done to meet the secondary objective of evaluation of preliminary evidence of hematologic response by documenting anti-leukemic activity in an heterogenous population of participants with various CD123-expressing hematologic malignancies. Anti-leukemic activity will be assessed as defined by the modified International Working Group (IWG) criteria for R/R-AML (for CRc rate) and HR-MDS (for CR rate) and using the NCCN criteria for B-ALL (for CRc rate).

In the adult and pediatric arms escalation Phase and expansion cohort C, efficacy assessment will be done to meet the secondary and tertiary (BPDCN only) objective of evaluation of preliminary evidence of hematologic response by documenting anti-leukemic activity in an heterogenous population of participants with various CD123-expressing hematologic malignancies. Anti-leukemic activity will be assessed as defined by the modified International Working Group (IWG) criteria for R/R AML (for CRc rate) and HR-MDS (for CR rate), the modified International Harmonization Project (IHP) lymphoma criteria for BPDCN (for rate of CR plus clinical CR, defined as complete response with residual skin abnormality not indicative of active disease, and overall response rate, ORR, defined as CR+clinical CR+CRi+PR), and using the NCCN criteria for B-ALL (for CRc rate).

In the adult/pediatric Expansion/Optimization Part Cohorts A, B and D, efficacy assessment will be done to meet the primary endpoint of assessing anti-leukemic activity of single agent CD123 NKCE at the RDE in participants with R/R AML. The primary endpoint for R/R AML will be composite complete remission (CRc) rate defined as the proportion of participants who have a CR (complete remission)+CRi (complete remission with incomplete hematologic recovery)+CRh (CR with partial hematologic recovery) per modified IWG for AML. For Cohort B the primary endpoint for HR-MDS will be ORR defined as the proportion of participants who have a CR+CR equivalent+PR+CR_{L}+CRh+HI) per IWG 2023.

Efficacy secondary objectives in the Expansion Part are included as an assessment of the

alternative CR rate in AML (Cohort A and D) (Proportion of participants with CR+CRh [complete remission with partial hematologic recovery], overall CR rate in AML (CR+CRh+CRi+MLFS [morphological
leukemia-free state]), duration of different types of responses (CR+CRi; CR+CRh; and CR+CRi+CRh+MLFS), event-free survival (EFS), rate of HSCT, time
to treatment failure (TTF), minimal residual disease (MRD) and survival rates at 6 months and 1 year. For Cohort B, the secondary endpoint will be alternative CR rate and the rate of HSCT. The secondary endpoint for Cohort C will be rate of CR+CRh+Cri for r/r AML and B-ALL as well as ORR rate for HR-MDS.

### Pharmacokinetics

Whole blood samples will be collected for measurement of plasma concentrations of CD123 NKCE as specified in the PK/Pdy flowcharts. The timing of sampling may be altered during the course of the study based on newly available data (eg, to obtain data closer to the time of peak plasma concentrations) to ensure appropriate monitoring.

The sampling schema for PK may be reduced during the course of the study based on the updated knowledge of drug behavior, upon notification from the Sponsor.

Instructions for the collection and handling of biological samples will be provided by the Sponsor or Sponsor's designee in a separate laboratory specifications document. The actual date and time of each sample will be recorded. Pharmacokinetic samples will be tested by the Sponsor or Sponsor's designee.

Plasma samples collected for determination of CD123 NKCE concentration may also be used to evaluate safety or efficacy aspects related to concerns arising during or after the study.

Pharmacokinetic samples could be used for testing analytical method performance such as comparability and incurred sample reproducibility. The exploratory data will not be included in the study report but will be kept on file.
During the course of the study, the pharmacokinetic parameters will be calculated using non-compartmental methods from plasma CD123 NKCE concentrations obtained after CD123 NKCE administration. The parameters will include, but may not be limited to, those listed in Table 19:

TABLE 19
List of pharmacokinetic parameters and definitions

| Parameters | Definition |
|---|---|
| Cₘₐₓ | Maximum observed concentration |
| tₘₐₓ | First time to reach Cₘₐₓ |
| C_{trough} | Concentration observed just before treatment |
| | administration during repeated dosing |

A population PK approach will be used for CD123 NKCE.

### Biomarkers

To identify biomarkers that help to predict and/or evaluate biological activities induced by CD123 NKCE, this study will evaluate exploratory biomarkers in bone marrow and blood.
Whole blood including derivatives (plasma and serum)
Bone marrow biopsy (solid)
Bone marrow aspiration (liquid)

Prospective real time analysis of critical biomarkers in freshly isolated samples will be used to evaluate safety, biological activity, association with the observed clinical responses (including duration of response, progression or early relapse), and to identify RDE for the expansion phase of the study. Samples will be tested for the following PD and response biomarkers:
Morphology and % blasts (including cytogenetics/FISH of bone marrow biopsy and/or aspiration) for evaluation of disease status and clinical responses (PD, SD, PR, CR, CRi, CRh)
Blast- and Immuno-phenotyping of blood and bone marrow aspiration: flow cytometry analysis of fresh whole blood and bone marrow aspiration samples will be performed for time course evaluation of leukemic blasts characteristics and depletion, including immunophenotyping to evaluate the impact of CD123 NKCE on the activation status and effector functions of relevant immune subsets including NK cells, T cells, and myeloid cells. CD123 prevalence and receptor density at baseline and after treatment will be summarized with descriptive statistics. Subpopulation analyses including effector/target ratio will be tabulated with standard descriptive statistics. Results will also be correlated with participants' clinical parameters. Additionally, flow cytometry-based AML MRD assays can be used in countries where Genomic/NGS-based AML MRD assays are unavailable to assess minimal residual disease and efficacy.
Cytokine responses in blood: to monitor pro-inflammatory cytokines including chemokines such as MIP-1β to assess CRS and PD responses. Individual concentrations of each measured cytokine will be tabulated and summarized by descriptive statistics (such as mean, geometric mean, median, SD, standard error of the mean, coefficient of variation, minimum, and maximum). Potential correlation with other parameters such as safety and pharmacokinetic endpoints will be also investigated. Results will be also correlated with participants' clinical parameters.
CRP/Ferritin in plasma for safety and inflammatory responses
CBC with differential of whole blood to monitor changes in leukemic blasts and blood leukocyte subpopulations.

Retrospective analysis will be performed on some additional selected biomarkers and data will be analyzed and summarized using standard descriptive statistics. Samples will be collected and stored for retrospective or batch analysis of biomarkers expected to play a key role in the MOA of CD123 NKCE including but not limited to:
Immunohistochemistry (IHC) of bone marrow biopsy: to evaluate expression of key targets (such as CD123/NKp46) and other relevant markers (PD-L1/2) at baseline to evaluate their association with observed clinical responses to CD123 NKCE and on therapy for PD responses. Results will be correlated with participants' clinical parameters.
Genomics and transcriptomics of bone marrow aspiration and whole blood: to evaluate biomarker variations and potential associations with clinical responses and benefit. This analysis will include CD16/CD32 genotyping, AML Mutational Profile (including MRD by next-generation sequencing), and RNAseq for genome-wide analysis of RNA signatures. Results will be correlated with participants' clinical parameters.
Baseline IgG in serum: for impact on PK and efficacy. Samples to be collected at screening or prior to first IMP administration. Results will be correlated with participants' clinical parameters.
Retrospective analysis of participant IHC and Genomics/Transcriptomics data, including MRD assessments, will be done on participant samples only during the expansion phase, unless needed to establish MRD methodology and facilitate selection of RDE at the end of the escalation phase. Optimal timepoints for analysis will be selected based on participants achieving CR.

Other samples may be used for research to develop methods, validate assays, prognostics and/or companion diagnostics related to CD123 NKCE, or other related NK cell engagers (NKCE) or T cell engagers (TCE). The goal of this work will be to investigate disease process, pathways associated with disease state, and/or mechanism of action of this class of NKCE, including but not limited to assessment of MRD, CD123 expression levels and E/T ratios of NK cells to AML blasts.

Samples collected for biomarker analyses and their derivatives may be stored for a period of up to 25 years after last participant last visit for potential re-analyses.

Immunogenicity Assessments: Antibodies to CD123 NKCE will be evaluated in plasma samples collected from all participants according to the PK/PDy flow-charts. Additionally, plasma samples should also be collected at the final visit from participants who discontinued study intervention or were withdrawn from the study. Instructions for the collection and handling of these samples will be provided by the Sponsor or Sponsor's designee in a separate laboratory specifications document. These samples will be tested by the Sponsor or Sponsor's designee, using a qualified assay method. A 3-tiered approach will be employed to assess the immunogenicity of CD123 NKCE when applicable. Samples will be screened and then confirmed for antibodies binding to CD123 NKCE and the titer of confirmed positive samples will be reported. Other analyses may be performed to further characterize the immunogenicity of CD123 NKCE. All samples collected for detection of antibodies to study intervention may also be evaluated for CD123 NKCE plasma concentration to enable interpretation of the immunogenicity data. The impact of positive immune response, including pre-existing, treatment-induced or treatment boosted ADA, will be evaluated on efficacy, pharmacokinetic, and safety endpoints when relevant.

### Tertiary Exploratory Analyses

Tertiary/exploratory endpoints include (but not limited to) immunophenotype of tumor and effector cells, and peripheral blood cytokine levels. Analyses will be described in the statistical analysis plan finalized before database lock.

The biomarker analyses will be performed using defined biomarker-specific populations. Pharmacodynamic biomarkers will be assessed separately for blood and bone marrow specimens. These will include summary and descriptive analyses (ie, time [longitudinal] and dose effect) for both CD123+ malignant (AML) and normal (immune cell) populations such as monocytes, etc, to determine proof of mechanism. Changes in relevant immune cell populations and respective kinetics in blood induced by CD123 NKCE will be analyzed through summary and descriptive analyses of immune cells such as CD8 and CD4 T-cells and NK cells. In parallel to the immunophenotyping by flow cytometry blood in exposed participants will also be analyzed by a comprehensive inflammatory cytokine panel. The assessment will include pre- and post-treatment level of these biomarkers at various time points after drug administration. Duplicate biomarker (more than one set of data for a particular visit) is not expected, except for parallel but independent immunophenotyping and hematologic evaluation of blasts and some basic leukocyte populations.

### Analysis of Pharmacodynamic Endpoints:

Pharmacodynamic assessments on blood and bone marrow will be performed, including:
To measure CD123 expression in malignant cells and kinetics of this expression under treatment.
To monitor CD123 expression on normal cells.
To assess minimal residual disease (MRD) in participants achieving a complete remission (CR) or complete remission with incomplete hematologic recovery (CM), and correlate MRD with clinical outcome.
To assess NK and T-cell subpopulations (eg, CD4, CD8) and activation status.
To investigate the relationship between CD123 expression, disease molecular subtype (as defined by marker expression, cytogenetics and/or genomics) and parameters of clinical response.
To assess levels of cytokines following treatment administration, and their relationship with safety profile and clinical outcome.

Findings from the pharmacodynamics markers will be descriptively summarized and tabulated.

### Example 9. Safety and Efficacy Data in Patients with r/r AML

### Methods

CD123 NKCE was administered intravenously twice weekly or once weekly (QW), depending on the dose level (DL) for the first 2 weeks of cycle 1 and then weekly for the rest of the induction cycles. Patients received approximately three 28-day induction cycles with patients achieving a complete remission (CR) or incomplete hematologic recovery (CRi) per International Working Group criteria transitioning to a 56-day maintenance period with dosing approximately every 29 days if not a candidate for stem cell transplantation. The primary objectives were to establish safety/tolerability and anti-leukemic activity (composite complete remission [CRc]=CR+CRi).

### Results

A total of 59 patients (58 with r/r AML; 1 with HR-MDS) across eleven DLs at 10-6000 μg/kg/dose (3 patients at DL1, 4 each at DL2-DL5 and DL2 mg, 6 each at DL0.75 mg and DL1.5 mg, 8 each in DL6-DL7 and DL1 mg) were included in the safety population. The median age was 67 years (range: 19-81). Patients had received a median of 2 (1-10) prior lines of treatment. The ECOG scores for these patients were all below ECOG 3 (ECOG 0, n=13 (22.03%). ECOG 1, n=43 (72.88%), ECOG 2, n=3 (5.08%) 18 patients (30.5%) reported receiving prior hematopoietic stem cell transplantation and 52 patients (88.1%) reporter prior exposure to venetoclax. The median white blood cell range at baseline was 2.6×10⁹/L (0-12×10⁹/L). The median blast count range in blood was 0.7×10⁹/L and median blast count range in bone marrow was 47.5% (1.0-90.0). 3 patients (5.2%) had extramedullary disease. Patients received a median of 2 cycles (range: 1-11) for a median duration of 7.9 weeks (range: 1-66). No dose limiting toxicities (DLTs) were observed up to the highest dose of 6000 μg/kg QW. The most common TRAE generally occurred during cycle 1/day 1 and typically resolved within 1 hour utilizing temporary interruption of infusion and symptom management. Grade ≥3 TRAEs were reported in 3 patients and included grade 3 diverticulitis in 1 patient in DL3 and grade 4 neutropenia in 1 patient in DL3 and grade 3 CRS in 1 patient. There were no cases of immune effector cell-associated neurotoxicity syndrome.
   Other TRAEs observed were decreased appetite, headache, diarrhea, nausea, anemia, chills, cytokine release syndrome, polyneuropathy, fatigue and inflammation.

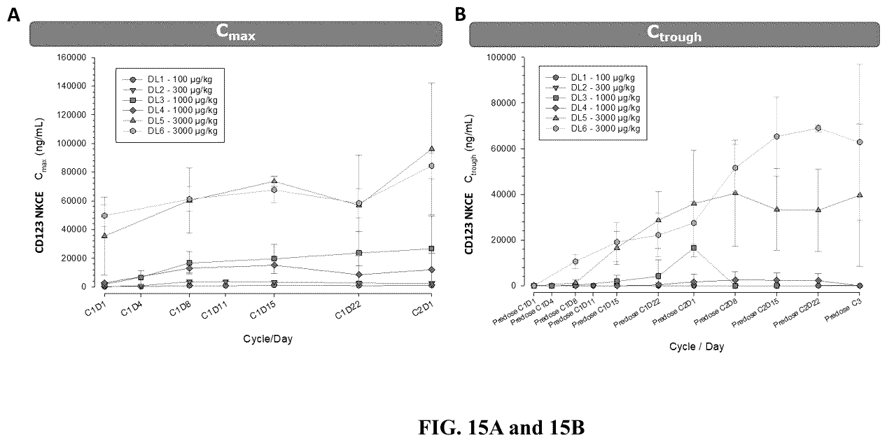

TEAEs were reported in 58 patients (98.3%) with grade ≥3 adverse events (AEs) in 40 patients (67.8%) and treatment-related AEs in 40 patients (67.8%), respectively. Serious TEAEs were reported in 35 pts (59.3%). TEAEs leading to the permanent discontinuation of CD123 NKCE were reported in 4 pts (6.8%). The most common TEAE was infusion-related reaction (n=33 [55.9%]) and constipation (n=13 [22%]), all being ≤grade 2 events. There were 4 cases of cytokine release syndrome (6.8%), 3 patients at grade 1 at 1 mg/kg, 1.5 mg/kg, and 3 mg/kg, and 1 patient at grade 3 at 0.75 mg/kg) and no case of immune effector cell-associated neurotoxicity syndrome. Other TEAEs observed were cholestasis, sepsis, lobular pneumonia, progressive disease, urosepsis, nontraumatic intracranial hemorrhage. Grade 5 TEAEs included cholestasis, nontraumatic intracranial hemorrhage, lobular pneumonia, lung sepsis, and urosepsis; all grade 5 events were unrelated to CD123 NKCE. The CRc rate was 8.6% (5/58 r/r AML patients evaluable for response). In DLs with a highest dose of 1000 μg/kg QW (DL3, DL4, DL1 mg), 5/15 (33.3%) patients achieved a CR (4 CR/1 CRi) as of the cutoff date. Complete remissions were observed upon completion of 1 (CR and CRi), 2, 3 and 4 cycles of treatment, and the median duration of CR/CRi was 48 weeks. The maximum time on treatment was 65 weeks. The median time to first CR/CRi was 16.1 weeks 95% confidence interval (8.3—Not estimable [NE]), and the median duration of CR/CRi is NE due to limited follow-up time. Two responders remain in remission after 8.8 and 13 months of treatment (FIG. 13A and FIG. 13B and FIG. 13C) with the longest duration of response ongoing at 13+ months and one patient receiving a transplant. 3 patients with CR (responders) are still receiving treatment. 4 responders had prior venetoclax and 2 responders had prior HSCT.

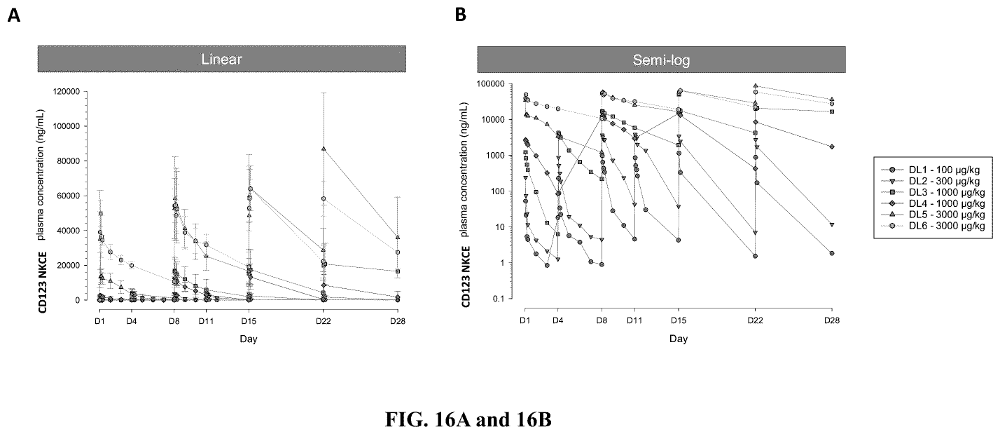

Further, leukemic blast numbers were measured at baseline (prior to CD123 NKCE treatment) and after treatment at dose levels DL1-DL7 in whole blood and in the bone marrow. Preliminary results demonstrate that treatment with the CD123 NKCE resulted in a reduction in blast count in both whole blood and the bone marrow (FIG. 14A-14D). AML blast reduction was observed across all CD123 NKCE dose levels. Preliminary results suggest that heterogenous effector cell: target cell (E/T) ratios were observed from variable patient blasts and NK cell counts, and there was modulation of peripheral NK cells observed at all doses tested. CD123 expression and a robust expression of NKp46 (>50%) were observed in all patients with a high variability of receptor density at baseline and changes on treatment.

Preliminary data indicates that CD123 NKCE was well tolerated up to doses of 6000 μg/kg QW with observed clinical benefit in patients with R/R AML. The observed clinical responses over a large range of CD123 NKCE doses demonstrated dose-dependent bell-shaped activity observed in patients that was reproduced with in vitro AML cell models. The maximum response was seen at a target dose of 1 mg/kg (1,000 μg/kg) (33% CR/CRi). The bell-shaped dose-response curve is consistent with modeling of predicted synapse formation and in vitro data shown below in Example 11. These data form the basis for selection of recommended doses for development in the phase 2 portion of the study.

### Example 10. Preliminary Pharmacokinetics (PK) and Pharmacodynamic (PD) Analysis of CD123 NKCE in Patients with r/r AML, B-ALL, or HR-MDS

### Methods

CD123 NKCE was administered intravenously twice weekly or once weekly (QW), depending on the per dose level (DL) for the first 2 weeks of cycle 1 and then QW for rest of induction cycles. Multiple peripheral blood (PB) samples and a bone marrow sample are collected during each induction cycle for PK profiles characterization and PD analysis. PB blood samples were used for the measurement of plasma concentrations of CD123 NKCE on Gyrolab platform for PK parameter calculation and to assess the immunogenicity (anti-drug antibodies [ADA]). Analysis of cytokines, as potential safety and PDy markers, including IL-6, TNFa and IFNg, was performed using a multiplex assay, Meso Scale Discovery® (MSD), based on the Human Proinflammatory Cytokine Panel 1 run on the Meso™ Sector Imager S600.

### Results

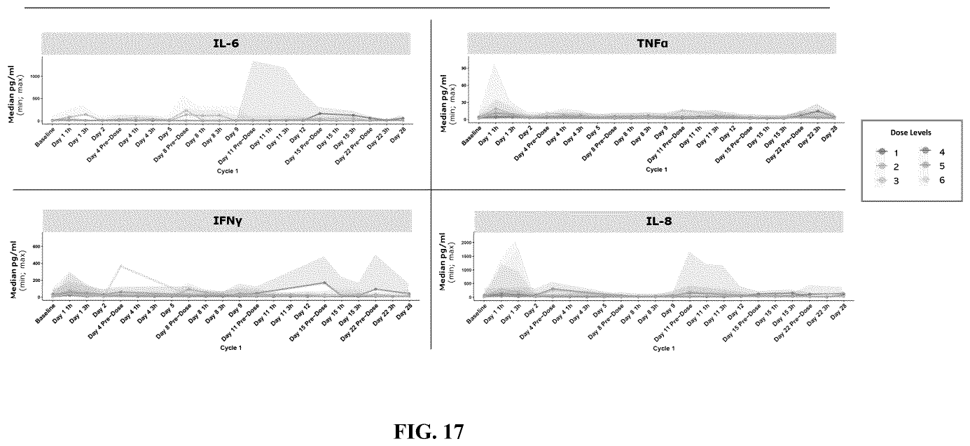

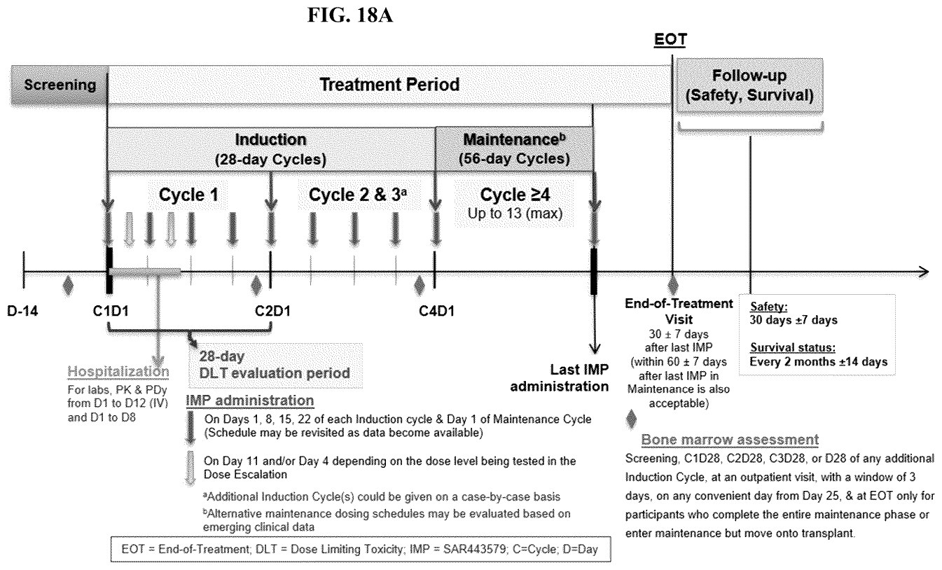

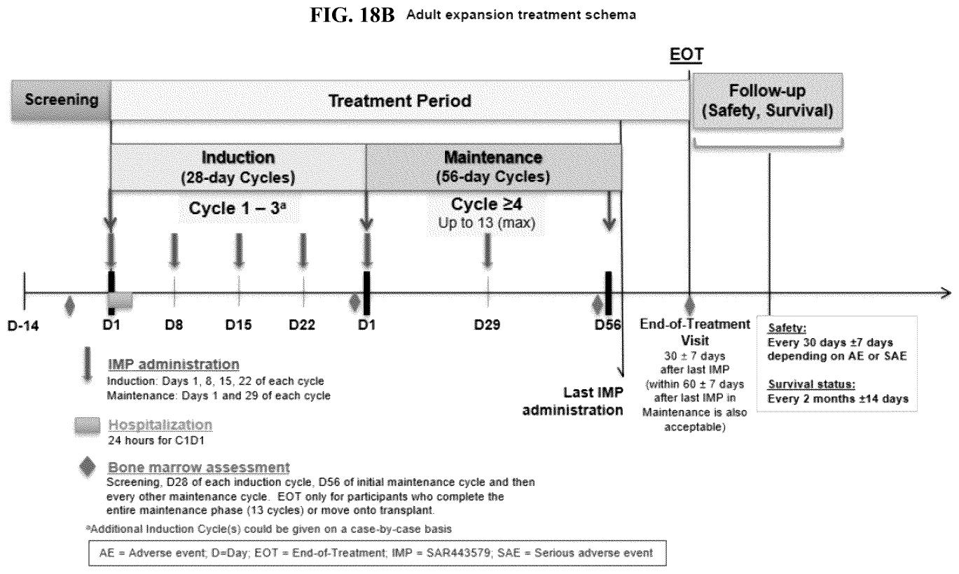

Over a period of about 10 months, 19 patients were treated across 5 DLs (3 patients in DL1 and 4 each in DL2-DL5) with escalating doses of CD123 NKCE between 10-3000 μg/kg/dose. No dose limiting toxicities (DLTs) were observed in any participant. At 1000 μg/kg QW, 3/8 (37.5%) patients achieved a CR (2 CR/1 CRi; incomplete hematologic recovery). Early immunogenicity incidence was of 26% with no impact on safety/efficacy. After the first administration of CD123 NKCE, circulating maximum concentration increased with dose increase with a supra-dose proportionality between DL1 and DL6 (FIG. 15A-15B). PK linearity was achieved at weekly doses of 3000 μg/kg (DL5/6) by the end of first cycle (FIG. 16A-16B). Median (maximum) peak cytokine concentrations (pg/mL) observed during DL1-5 are shown in Table 20 and FIG. 17.

TABLE 20
Median peak cytokine concentrations (pg/mL). Maximum peak
concentrations are in parentheses.

| Cytokine | DL1 | DL2 | DL3 | DL4 | DL5 |
|---|---|---|---|---|---|
| IL-6 | 63.6 | 118.3 | 62.7 | 29.7 | 52.3 (445.3) |
| | (1355.4) | (264.3) | (450.1) | (126.7) | |
| TNFα | 12.6 (14.7) | 19.2 (88.0) | 9.7 (12.1) | 7.2 (11.9) | 17.9 (27.4) |
| IFNγ | 21.4 (50.7) | 88.7 | 81.8 | 123.8 | 58.1 (256.6) |
| | | (233.4) | (152.8) | (420.4) | |

Clinical responses or achievement of linear PK exposure were not associated with exacerbated peak cytokine levels. CRS levels of IL-6 (>500/1000 μg/mL) were only measured in a single DL-1 patient.

CD123 NKCE was well tolerated up to doses of 3000 μg/kg QW with observed clinical benefit in patients with R/R AML. The observed peak cytokine levels did not show significant dose-related increases and, importantly, there was no association between elevated peak cytokine levels and clinical responses. These results are consistent with the improved safety profile of this CD123-targeted NKCE compared to similar CD123-targeted T-cell engagers. In summary, CD123 NKCE induced transient cytokine responses at all dose levels tested, which remained outside of reported CRS ranges. Continued enrollment and longer follow-up of patients on CD123 NKCE will further extend this work with longitudinal cytokine data analysis exploring additional dose dependent effects and associations with clinical safety and efficacy. PK linearity of CD123 NKCE was achieved at weekly dosing of 3000 μg/kg.

### Example 11. CD123 NKCE Dose Response Efficacy Mechanisms in Acute Myeloid Leukemia (AML) Models

### Methods

The cytotoxic activity of the CD123 NKCE was evaluated in vitro over a broad range of concentrations against different AML cell models exhibiting low (OCI-AML2 and NB-4 cells, ˜1300 and 2500 sites/cells, respectively), medium (THP-1 cells. ˜6000-9000 sites/cells) or high (MOLM-13 cells, ˜11000-20000 sites/cells) CD123 receptor density in the presence of healthy donor NK cells at different Effector (E) to Target (T) (E:T) ratios and different time points. Cytotoxic activity was measured using the Calcein-AM release assay, the Incucyte live-cell imaging analysis system or by flow cytometry measuring tumor cell viability after Cell Trace Violet (CTV)-Propidium iodide (PI) staining. The effect of the CD123 NKCE on NK cell activation was evaluated by flow cytometry. In vivo, the anti-tumor activity of a surrogate CD123 NKCE antibody which binds to murine NKp46 on mouse NK cells was tested in SCID mice intravenously (IV) injected with human MOLM-13 AML cells. The surrogate CD123 NKCE antibody was tested at doses ranging from 0.05 to 30 mg/kg intraperitoneal single administrations.

### Results

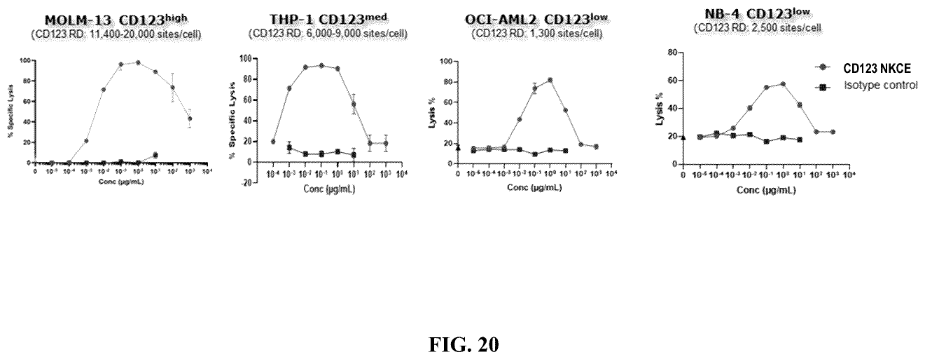

In vitro, the CD123 NKCE demonstrated potent cytotoxic activity in an E:T ratio and time-dependent manner against all 4 AML cell models tested, whatever the CD123 receptor density level. In all AML cell lines, a bell-shaped concentration-dependent cytotoxic activity curve was observed upon treatment with the CD123 NKCE. Interestingly, for all cell lines, the maximum cytotoxic effect was reached within the same range of concentrations (ECmax range from 0.08 to 0.66 μg/ml geometric mean), irrespective of the E:T ratio, the duration of treatment and the level of CD123 expression (FIG. 20).

In vivo, the CD123 NKCE surrogate NK cell engager also demonstrated potent anti-tumor efficacy with a bell-shaped dose-dependent activity and decreased activity at the 2 highest doses tested, 10 and 30 mg/kg.

### Conclusions

The CD123 NKCE demonstrates potent anti-tumor activity against AML tumor cells expressing low, medium or high CD123 antigen densities, with a bell-shaped concentration-effect up to high concentrations in vitro, regardless of CD123 receptor density or E:T ratio. A similar bell-shaped dose-effect was observed in an in vivo mouse model.

### Example 12. Phase 2 CD123 NKCE Dose Determination

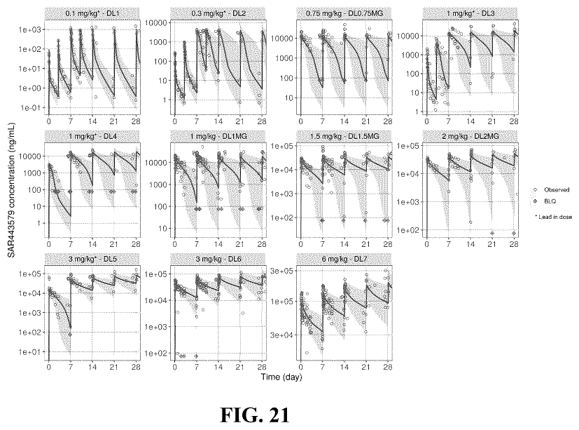

A population PK model was developed using CD123 NKCE plasma concentrations vs time data from 58 out of 59 AML participants treated at doses from 0.01 up to 6 mg/kg during the induction phase of the monotherapy dose escalation part. The PK of CD123 NKCE was best characterized using a two-compartments distribution model with parallel linear and non-linear elimination [full TMDD model]. The PK model adequately described the observed CD123 NKCE concentrations vs time data and enabled to capture both the non-linear and the linear PK profiles which dominate at dose levels ≥1.5 mg/kg (FIG. 21).

The population PK analyses refinement with these new clinical data have continued to support a once weekly schedule during induction period, but the data indicated more frequent administration of CD123 NKCE during the maintenance period from Q8W to Q4W.

Leveraging in vitro cytotoxic data, a synapse model integrating CD123 NKCE binding with NKp46 and CD123 receptors was linked to the PK model. As shown from in vitro data, the maximum cytotoxic effect was reached within the same range of CD123 NKCE concentrations (0.08 to 0.66 μg/ml), irrespective of the E:T ratio, the duration of treatment or the level of CD123 expression. Based on those findings, the developed synapse PK model predicted maximal synapse concentrations leading to maximal cytotoxicity effect within an optimal range of CD123 NKCE concentrations corresponding to dissociation constant values (experimental in vitro KD) of CD123 NKCE binding with effector (NKp46+) and target (CD123+) cells.

The synapse PK model was able to correctly capture the range of concentrations leading to maximal cytotoxic effect without considering low affinity binding of CD123 NKCE with CD16+ receptors. Therefore, the binding of CD123 NKCE to CD16+ cells was neglected in the synapse model.

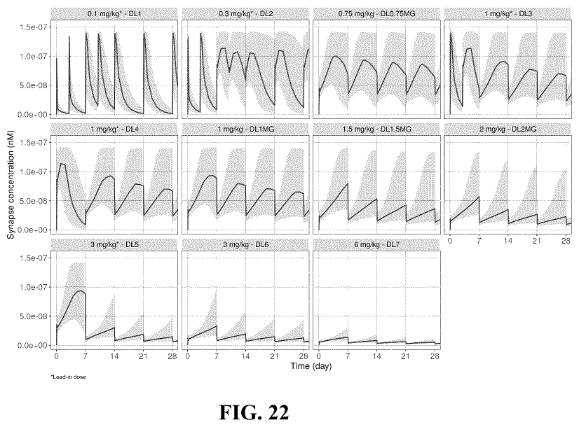

This synapse PK model was translated in human to predict tripartite complex formation considering observed clinical PK data, effectors and target cells blood concentrations measured in participants at baseline and incorporating in vitro KD values for NKp46 and CD123 receptors. Simulated time course profiles of synapse concentrations showed maximal synapse concentrations roughly from 0.3 mg/kg up to 1 mg/kg while synapse formation was decreased for dose levels ≥1.5 mg/kg (FIG. 22).

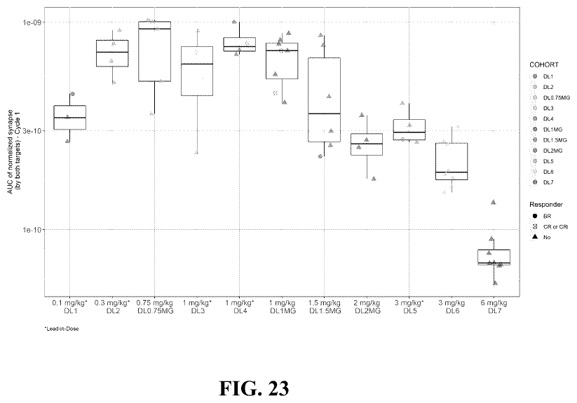

Synapse concentrations were normalized to effectors and target cells (tripartite complex concentration per cells) to dissociate drug effect (CD123 NKCE concentrations) from confounding prognostic factors (e.g CD123 positive blast count, NKp46 positive cells count, E/T ratio). Cumulated exposure of synapse concentrations normalized per cells over the first induction cycle (AUC_{0-28 days}) were predicted in all patients and demonstrated a bell-shape curve over the range of tested clinical dose levels. This bell-shape pattern was in line with the observed dose-response relationships from which all CR/CRi were observed at 1 mg/kg dose range (FIG. 23).

### Synapse Exposure Versus Clinical Response Analysis

Synapse Exposure-Response analysis using multivariate logistic regression modeling showed that synapse exposure (cumulated AUC_{0-28 days} of synapse concentrations normalized per cells over the first induction cycle) was positively correlated (p<0.05) with the clinical response (CR/CRi or BR). Of note, similar E/R analysis considering CD123 NKCE plasma PK exposure instead of synapse PK exposure did not reach any significant correlation with clinical response.

Based on E/R model, trials simulations were performed to predict the response rate across dose levels: 1000 trials of 58 patients randomly sampled with replacement from the original dataset were simulated for each dose level and accounted for E/R model parameters uncertainty.

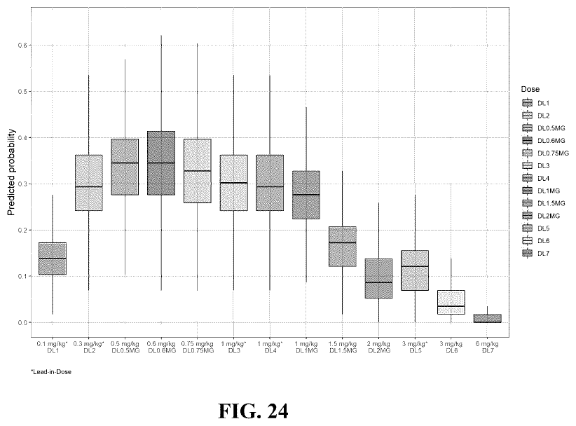

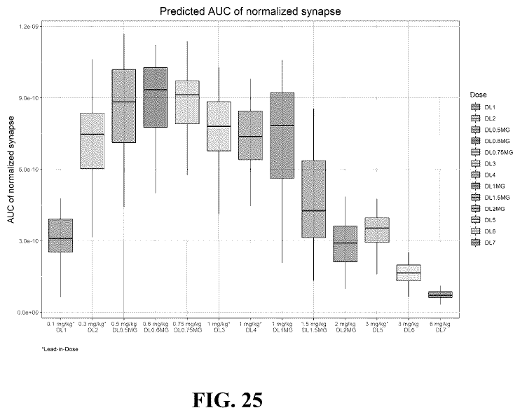

Simulations showed that probability of maximal response rate (CR/CRi or BR) close to 30% in average was achieved from 0.3 mg/kg (DL2) up to 1 mg/kg (DL3, DL4 and DL1 mg), corresponding to the range of maximal synapse exposure predicted by the PK-Synapse model (FIG. 24). Simulations of synapse PK exposure showed that maximal synapse exposure was achieved for dose levels from 0.3 to 1 mg/kg (FIG. 25).

A dose of 1 mg/kg QW was first selected for dose optimization since all CR/CRi were observed at DLs using a target dose of 1 mg/kg (DL3/DL4/DL1 mg).

Additionally, based on the above simulations, a dose of 0.6 mg/kg QW for the induction regimen was selected for further testing for dose optimization. This dose regimen aimed to achieve maximal synapse exposure with a lower CD123 NKCE plasma exposure compared to the 1 mg/kg dose level (i.e. 3-fold lower CD123 NKCE plasma AUC0-28 days at 0.6 mg/kg compared to 1 mg/kg).

PK-Synapse and PK/PD analyses supported dose optimization of CD123 NKCE in AML adult patients, with the selection of a lower dose regimen expected to maximize synapse exposure and efficacy while reducing significantly CD123 NKCE plasma exposure.

However, given the limited sample size and considering model uncertainty at this stage of clinical development, it is also proposed to evaluate an additional higher dose of 1.5 mg/kg QW (i.e. 2-fold higher CD123 NKCE plasma AUC_{0-28 days} at 1.5 mg/kg compared to 1 mg/kg) for dose optimization. Overall, two additional doses (i.e.: 0.6 and 1.5 mg/kg/QW) on top of 1 mg/kg QW would increase confidence for a full assessment of the bell-shape dose and exposure-response relationships.

### Conclusion

The 1 mg/kg dose was chosen for further clinical assessment due to initial positive clinical responses and a manageable safety profile. To refine doses around 1 mg/kg to consider for optimization, an exposure-response (E/R) model was developed leveraging in-vitro cytotoxic data and integrating clinical response and synapse PK exposure data. This model suggested that a dose of 0.6 mg/kg may achieve comparable efficacy to 1 mg/kg but with three times lower CD123 NKCE PK exposure. However, due to limited sample size and model uncertainty in this early stage of clinical development, the sponsor is also planning to evaluate the dose of 1.5 mg/kg in order to evaluate a dose range that extends above.

In summary, PK-Synapse and ER modeling, built with in vitro and clinical data, recommends 0.6 mg/kg once a week (QW) during the induction period. The observed clinical responses would support further investigation of the 1 mg/kg dose. Finally, acknowledging the limited dataset at this stage of development, the sponsor proposes an additional dosage of 1.5 mg/kg QW to enhance confidence and thoroughly evaluating dose and exposure versus response relationships at 0.6 mg/kg, 1 mg/kg and 1.5 mg/kg.

1. A method of treating or preventing a leukemia or a myelodysplastic syndrome in a subject in need thereof, the method comprising administering to the subject a binding protein comprising a first antigen binding domain with binding specificity to CD123 and a second antigen binding domain with binding specificity to NKp46, wherein the binding protein is administered to the subject at a dose of at least 3 μg/kg.

2. (canceled)

3. The method of claim 1, wherein the dose is between about 3 μg/kg and about 6000 μg/kg; optionally wherein the dose is:
between about 3 μg/kg and about 30 μg/kg;
between about 10 μg/kg and 100 μg/kg;
between about 30 μg/kg and 100 μg/kg;
between about 13 μg/kg and about 130 μg/kg;
between about 15 μg/kg and about 150 μg/kg;
between about 20 μg/kg and about 200 μg/kg;
between about 30 μg/kg and about 300 μg/kg;
between about 40 μg/kg and about 400 μg/kg;
between about 45 μg/kg and about 450 μg/kg;
between about 60 μg/kg and about 600 μg/kg;
between about 100 μg/kg and about 1000 μg/kg;
between about 300 μg/kg and about 1000 μg/kg;
between about 400 μg/kg and about 1300 μg/kg;
between about 450 μg/kg and about 1500 μg/kg;
between about 600 μg/kg and about 1000 μg/kg;
between about 600 μg/kg and about 1500 μg/kg;
between about 600 μg/kg and about 2000 μg/kg;
between about 1000 μg/kg and about 3000 μg/kg;
between about 1300 μg/kg and about 3000 μg/kg;
between about 1500 μg/kg and about 3000 μg/kg; or
between about 2000 μg/kg and about 3000 μg/kg.

4-24. (canceled)

25. The method according to claim 1, wherein the dose is about 3 μg/kg; about 10 μg/kg; about 13 μg/kg; about 15 μg/kg; about 20 μg/kg; about 30 μg/kg; about 40 μg/kg; about 50 μg/kg; about 60 μg/kg; about 100 μg/kg; about 130 μg/kg; about 150 μg/kg; about 200 μg/kg; about 300 μg/kg; about 400 μg/kg; about 450 μg/kg; about 600 μg/kg; about 750 μg/kg; about 1000 μg/kg; about 1300 μg/kg; about 1500 μg/kg; about 2000 μg/kg; about 3000 μg/kg; about 4000 μg/kg; about 4500 μg/kg; or about 6000 μg/kg.

26-51. (canceled)

52. The method according to claim 1, wherein the binding protein is administered to the subject intravenously, subcutaneously, intraperitoneally, or intramuscularly; optionally wherein the binding protein is administered:
to the subject intravenously;
to a subject for at least one cycle, optionally wherein the at least one cycle is about 28 days;
to a subject for at least two cycles; and/or
to a subject for three or more cycles.

53-57. (canceled)

58. The method of claim 1, wherein the first antigen binding domain with binding specificity to CD123 comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively; optionally wherein:
a. the VH1 comprises an amino acid sequence that is at least about 90% identical to the amino acid sequence of SEQ ID NO: 41, and wherein the VL1 comprises an amino acid sequence that is at least about 90% identical to the amino acid sequence of SEQ ID NO: 43; or
b. the VH1 comprises an amino acid sequence that is at least about 90% identical to the amino acid sequence of SEQ ID NO: 42, and wherein the VL1 comprises an amino acid sequence that is at least about 90% identical to the amino acid sequence of SEQ ID NO: 44; and/or
c. the VH1 comprises an amino acid sequence of SEQ ID NO: 41, and wherein the VL1 comprises an amino acid sequence of SEQ ID NO: 43; or
d. the VH1 comprises an amino acid sequence of SEQ ID NO: 42, and wherein the VL1 comprises an amino acid sequence of SEQ ID NO: 44.

59-60. (canceled)

61. The method of claim 1, wherein the second antigen binding domain with binding specificity to NKp46 comprises:
a. a second heavy chain variable domain (VH2) comprising a CDR-H1, H2, and H3 corresponding to the amino acid sequences of:
i. SEQ ID NO: 13, 14, and 15, respectively;
ii. SEQ ID NO: 16, 17, and 18, respectively;
iii. SEQ ID NO: 19, 20, and 21, respectively;
iv. SEQ ID NO: 22, 23, and 24, respectively; or
v. SEQ ID NO: 16, 25, and 26, respectively; and
b. a second light chain variable domain (VL2) comprising a CDR-L1, L2, and L3 corresponding to the amino acid sequences of:
i. SEQ ID NO: 27, 28, and 29, respectively;
ii. SEQ ID NO: 30, 31, and 32, respectively;
iii. SEQ ID NO: 33, 34, and 35, respectively;
iv. SEQ ID NO: 36, 37, and 38, respectively; or
v. SEQ ID NO: 39, 31, and 40, respectively; optionally wherein:
a. the VH2 comprises an amino acid sequence that is at least about 90% identical to the amino acid sequence of SEQ ID NO: 45, and wherein the VL1 comprises an amino acid sequence that is at least about 90% identical to the amino acid sequence of SEQ ID NO: 53;
b. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54;
c. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
d. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
e. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
f. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
g. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
h. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60;
and/or
i. the VH2 comprises an amino acid sequence of SEQ ID NO: 45, and wherein the VL1 comprises an amino acid sequence of SEQ ID NO: 53;
j. the VH2 comprises an amino acid sequence of SEQ ID NO: 46, and wherein the VL2 comprises an amino acid sequence of SEQ ID NO: 54;
k. the VH2 comprises an amino acid sequence of SEQ ID NO: 47, and wherein the VL2 comprises an amino acid sequence of SEQ ID NO: 55;
l. the VH2 comprises an amino acid sequence of SEQ ID NO: 48, and wherein the VL2 comprises an amino acid sequence of SEQ ID NO: 56;
m. the VH2 comprises an amino acid sequence of SEQ ID NO: 49, and wherein the VL2 comprises an amino acid sequence of SEQ ID NO: 57;
n. the VH2 comprises an amino acid sequence of SEQ ID NO: 50, and wherein the VL2 comprises an amino acid sequence of SEQ ID NO: 58;
o. the VH2 comprises an amino acid sequence of SEQ ID NO: 51, and wherein the VL2 comprises an amino acid sequence of SEQ ID NO: 59; or
p. the VH2 comprises an amino acid sequence of SEQ ID NO: 52, and wherein the VL2 comprises an amino acid sequence of SEQ ID NO: 60.

62-63. (canceled)

64. The method according to claim 1, wherein the binding protein comprises three polypeptide chains (I), (II) and (III) that form two ABDs, as defined below:
V_{1A}-C_{1A}-Hinge₁-(C_{H}2-C_{H}3)_{A} (I)
V_{1B}-C_{1B}-Hinge₂-(C_{H}2-C_{H}3)_{B}-L₁-V_{2A}-C_{2A}-Hinge₃ (II)
V_{2B}-C_{2B} (III)
wherein
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C1 (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker; optionally wherein:
C_{1B} is an immunoglobulin heavy chain constant domain 1 (C_{H}1),
C_{2A} is an immunoglobulin heavy chain constant domain 1 (C_{H}1),
C_{L} corresponds to an immunoglobulin kappa light chain constant domain (C_{κ});
(C_{H}2-C_{H}3)_{A} corresponds to the amino acid sequence of SEQ ID NO: 69;
(C_{H}2-C_{H}3)_{B} corresponds to the amino acid sequence of SEQ ID NO: 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

65. (canceled)

66. The method of claim 1, wherein residue N297 of the Fc region or variant thereof according to EU numbering comprises a N-linked glycosylation; or the all or part of the Fc region or variant thereof binds to a human CD16A (FcγRIII) polypeptide; optionally wherein:
at least two polypeptide chains are linked by at least one disulfide bridge, optionally wherein the polypeptide chains (I) and (II) are linked by at least one disulfide bridge between C_{1A} and Hinge₂ and/or wherein the polypeptide chains (II) and (III) are linked by at least one disulfide bridge between Hinge₃ and C_{2B};
V_{1A} is V_{L1} and V_{1B} is V_{H1}; and/or
V_{2A} is V_{H2} and V_{2B} is V_{L2}.

67-71. (canceled)

72. The method of claim 1, wherein:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(a) V_{H1} and V_{L1} corresponds to the amino acid sequences of SEQ ID NO: 41 and 43 respectively or corresponds to the amino acid sequences of SEQ ID NO: 42 and 44 respectively; and/or
(b) V_{H2} and V_{L2} corresponds to
the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively; and/or
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
(i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53:
(j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58;
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or
V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60; and/or
polypeptide (I) comprises an amino acid sequence of SEQ ID NO: 64;
polypeptide (II) comprises an amino acid sequence of SEQ ID NO: 65; and
polypeptide (III) comprises an amino acid sequence of SEQ ID NO: 66.

73-75. (canceled)

76. The method of claim 1, wherein:
the leukemia is:
acute myeloid leukemia (AML); optionally wherein the AML is relapsed or refractory; and/or
B cell acute lymphoblastic leukemia (B-ALL); and/or;
the myelodysplastic syndrome is a high-risk myelodysplastic syndrome (HR-MDS).

77-83. (canceled)

84. A method of treating or preventing a leukemia or a hematological neoplasm in a pediatric subject in need thereof, the method comprising administering to the subject a binding protein comprising a first antigen binding domain with binding specificity to CD123 and a second antigen binding domain with binding specificity to NKp46.

85. A method of treating or preventing blastic plasmacytoid dendritic cell neoplasm (BPDCN) in a subject in need thereof, the method comprising administering to the subject a binding protein comprising a first antigen binding domain with binding specificity to CD123 and a second antigen binding domain with binding specificity to NKp46.

86. The method of claim 85, wherein the first antigen binding domain comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively.

87. The method of claim 84, wherein:
the subject is a pediatric subject;
the binding protein is administered to the subject intravenously, subcutaneously, intraperitoneally, or intramuscularly, optionally wherein the binding protein is administered to the subject intravenously;
the binding protein is administered at a dose between about 500 μg/kg and about 12,000 μg/kg;
the binding protein is administered at a dose between about 600 μg/kg and about 1000 μg/kg; and/or
the binding protein is administered at a dose between about 600 μg/kg and about 1500 μg/kg.

88-92. (canceled)

93. The method according to claim 87, wherein the dose is about 500 μg/kg; about 1000 μg/kg; about 1500 μg/kg; about 3000 μg/kg; about 6000 μg/kg; or about 12000 μg/kg.

94-99. (canceled)

100. The method claim 84, wherein the first antigen binding domain with binding specificity to CD123 comprises:
a heavy chain variable domain (VH1) comprising a CDR-H1, H2, and H3 corresponding to the amino acid sequences of SEQ ID NO: 1, 2, and 3, respectively or corresponding to the amino acid sequences of SEQ ID NO: 4, 5, and 5, respectively; and
a light chain variable domain (VL1) comprising a CDR-L1, L2, and L3 corresponding to the amino acid sequences of SEQ ID NO: 7, 8, and 9, respectively or corresponding to the amino acid sequences of SEQ ID NO: 10, 11, and 12, respectively; optionally wherein:
a. the VH1 comprises an amino acid sequence that is at least about 90% identical to the amino acid sequence of SEQ ID NO: 41, and wherein the V_{L1} comprises an amino acid sequence that is at least about 90% identical to the amino acid sequence of SEQ ID NO: 43; or
b. the VH1 comprises an amino acid sequence that is at least about 90% identical to the amino acid sequence of SEQ ID NO: 42, and wherein the V_{L1} comprises an amino acid sequence that is at least about 90% identical to the amino acid sequence of SEQ ID NO: 44; and/or
a. the VH1 comprises an amino acid sequence of SEQ ID NO: 41, and wherein the VL1 comprises an amino acid sequence of SEQ ID NO: 43; or
b. the VH1 comprises an amino acid sequence of SEQ ID NO: 42, and wherein the VL1 comprises an amino acid sequence of SEQ ID NO: 44.

101-102. (canceled)

103. The method of claim 84, wherein the second antigen binding domain with binding specificity to NKp46 comprises:
a. a second heavy chain variable domain (VH2) comprising a CDR-H1, H2, and H3 corresponding to the amino acid sequences of:
i. SEQ ID NO: 13, 14, and 15, respectively;
ii. SEQ ID NO: 16, 17, and 18, respectively;
iii. SEQ ID NO: 19, 20, and 21, respectively;
iv. SEQ ID NO: 22, 23, and 24, respectively; or
v. SEQ ID NO: 16, 25, and 26, respectively; and
b. a second light chain variable domain (VL2) comprising a CDR-L1, L2, and L3 corresponding to the amino acid sequences of:
i. SEQ ID NO: 27, 28, and 29, respectively;
ii. SEQ ID NO: 30, 31, and 32, respectively;
iii. SEQ ID NO: 33, 34, and 35, respectively;
iv. SEQ ID NO: 36, 37, and 38, respectively; or
v. SEQ ID NO: 39, 31, and 40, respectively;
c. the VH2 comprises an amino acid sequence that is at least about 90% identical to the amino acid sequence of SEQ ID NO: 45, and wherein the VL2 comprises an amino acid sequence that is at least about 90% identical to the amino acid sequence of SEQ ID NO: 53;
d. the VH2 comprises an amino acid sequence that is at least about 90% identical to the amino acid sequence of SEQ ID NO: 46, and wherein the VL2 comprises an amino acid sequence that is at least about 90% identical to the amino acid sequence of SEQ ID NO: 54;
e. the VH2 comprises an amino acid sequence that is at least about 90% identical to the amino acid sequence of SEQ ID NO: 47, and wherein the VL2 comprises an amino acid sequence that is at least about 90% identical to the amino acid sequence of SEQ ID NO: 55;
f. the VH2 comprises an amino acid sequence that is at least about 90% identical to the amino acid sequence of SEQ ID NO: 48, and wherein the VL2 comprises an amino acid sequence that is at least about 90% identical to the amino acid sequence of SEQ ID NO: 56;
g. the VH2 comprises an amino acid sequence that is at least about 90% identical to the amino acid sequence of SEQ ID NO: 49, and wherein the VL2 comprises an amino acid sequence that is at least about 90% identical to the amino acid sequence of SEQ ID NO: 57;
h. the VH2 comprises an amino acid sequence that is at least about 90% identical to the amino acid sequence of SEQ ID NO: 50, and wherein the VL2 comprises an amino acid sequence that is at least about 90% identical to the amino acid sequence of SEQ ID NO: 58;
i. the VH2 comprises an amino acid sequence that is at least about 90% identical to the amino acid sequence of SEQ ID NO: 51, and wherein the VL2 comprises an amino acid sequence that is at least about 90% identical to the amino acid sequence of SEQ ID NO: 59; or
j. the VH2 comprises an amino acid sequence that is at least about 90% identical to the amino acid sequence of SEQ ID NO: 52, and wherein the VL2 comprises an amino acid sequence that is at least about 90% identical to the amino acid sequence of SEQ ID NO: 60;
k. the VH2 comprises an amino acid sequence of SEQ ID NO: 45, and wherein the VL2 comprises an amino acid sequence of SEQ ID NO: 53;
l. the VH2 comprises an amino acid sequence of SEQ ID NO: 46, and wherein the VL2 comprises an amino acid sequence of SEQ ID NO: 54;
m. the VH2 comprises an amino acid sequence of SEQ ID NO: 47, and wherein the VL2 comprises an amino acid sequence of SEQ ID NO: 55;
n. the VH2 comprises an amino acid sequence of SEQ ID NO: 48, and wherein the VL2 comprises an amino acid sequence of SEQ ID NO: 56;
o. the VH2 comprises an amino acid sequence of SEQ ID NO: 49, and wherein the VL2 comprises an amino acid sequence of SEQ ID NO: 57;
p. the VH2 comprises an amino acid sequence of SEQ ID NO: 50, and wherein the VL2 comprises an amino acid sequence of SEQ ID NO: 58;
q. the VH2 comprises an amino acid sequence of SEQ ID NO: 51, and wherein the VL2 comprises an amino acid sequence of SEQ ID NO: 59; or
r. the VH2 comprises an amino acid sequence of SEQ ID NO: 52, and wherein the VL2 comprises an amino acid sequence of SEQ ID NO: 60.

104-105. (canceled)

106. The method of claim 84, wherein the binding protein comprises three polypeptide chains (I), (II) and (III) that form two ABDs, as defined below:
V_{1A}-C_{1A}-Hinge₁-(C_{H}2-C_{H}3)_{A} (I)
V_{1B}-C_{1B}-Hinge₂-(C_{H}2-C_{H}3)_{B}-L₁-V_{2A}-C_{2A}-Hinge₃ (II)
V_{2B}-C_{2B} (III)
wherein
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (C_{H}1/C_{L}) and C_{2A} and C_{2B} form a pair C₂ (C_{H}1/C_{L}) wherein C_{H}1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(C_{H}2-C_{H}3)_{A} and (C_{H}2-C_{H}3)_{B} are identical or different, and comprise an immunoglobulin heavy chain constant domain 2 (C_{H}2) and an immunoglobulin heavy chain constant domain 3 (C_{H}3);
L₁ is an amino acid linker; optionally wherein:
C_{1B} is an immunoglobulin heavy chain constant domain 1 (C_{H}1);
C_{2A} is an immunoglobulin heavy chain constant domain 1 (C_{H}1);
C_{L} corresponds to an immunoglobulin kappa light chain constant domain (C_{κ});
(C_{H}2-C_{H}3)_{A} corresponds to the amino acid sequence of SEQ ID NO; 69;
(C_{H}2-C_{H}3)_{B} corresponds to the amino acid sequence of SEQ ID NO; 70;
Hinge₁ corresponds to the amino acid sequence of SEQ ID NO:74;
Hinge₂ corresponds to the amino acid sequence of SEQ ID NO:75;
Hinge₃ corresponds to the amino acid sequence of SEQ ID NO: 77;
L₁ corresponds to the amino acid sequence of SEQ ID NO: 76.

107. (canceled)

108. The method of claim 84, wherein:
residue N297 of the Fc region or variant thereof according to EU numbering comprises a N-linked glycosylation;
the all or part of the Fc region or variant thereof binds to a human CD16A (FcγRIII) polypeptide;
the method comprises at least two polypeptide chains linked by at least one disulfide bridge, optionally wherein the polypeptide chains (I) and (II) are linked by at least one disulfide bridge between C1A and Hinge2 and/or wherein the polypeptide chains (II) and (III) are linked by at least one disulfide bridge between Hinge3 and C2B;
V_{1A} is V_{L1} and V_{1B} is V_{H1}; and/or
V_{2A} is V_{H2} and V_{2B} is V_{L2}.

109-113. (canceled)

114. The method of claim 84, wherein:
(a) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(b) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(c) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(d) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38;
(e) V_{H1} comprises a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; V_{L1} comprises a CDR-L1 comprising the amino acid sequence of SEQ ID NO: 7; a CDR-L2 comprising the amino acid sequence of SEQ ID NO: 8; a CDR-L3 comprising the amino acid sequence of SEQ ID NO: 9; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40;
(f) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 13; a CDR-H2 comprising the amino acid sequence of SEQ ID NO: 14; a CDR-H3 comprising the amino acid sequence of SEQ ID NO: 15; V_{L2} comprises a CDR-L1 comprising the amino acid sequence of SEQ ID NO: 27; a CDR-L2 comprising the amino acid sequence of SEQ ID NO: 28; a CDR-L3 comprising the amino acid sequence of SEQ ID NO: 29;
(g) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 17; a CDR-H3 comprising the amino acid sequence of SEQ ID NO: 18; V_{L2} comprises a CDR-L1 comprising the amino acid sequence of SEQ ID NO: 30; a CDR-L2 comprising the amino acid sequence of SEQ ID NO: 31; a CDR-L3 comprising the amino acid sequence of SEQ ID NO: 32;
(h) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 19; a CDR-H2 comprising the amino acid sequence of SEQ ID NO: 20; a CDR-H3 comprising the amino acid sequence of SEQ ID NO: 21; V_{L2} comprises a CDR-L1 comprising the amino acid sequence of SEQ ID NO: 33; a CDR-L2 comprising the amino acid sequence of SEQ ID NO: 34; a CDR-L3 comprising the amino acid sequence of SEQ ID NO: 35;
(i) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 22; a CDR-H2 comprising the amino acid sequence of SEQ ID NO: 23; a CDR-H3 comprising the amino acid sequence of SEQ ID NO: 24; V_{L2} comprises a CDR-L1 comprising the amino acid sequence of SEQ ID NO: 36; a CDR-L2 comprising the amino acid sequence of SEQ ID NO: 37; a CDR-L3 comprising the amino acid sequence of SEQ ID NO: 38; or
(j) V_{H1} comprises a CDR-H1 comprising the amino acid sequence of SEQ ID NO: 4; a CDR-H2 comprising the amino acid sequence of SEQ ID NO: 5; a CDR-H3 comprising the amino acid sequence of SEQ ID NO: 6; V_{L1} comprises a CDR-L1 comprising the amino acid sequence of SEQ ID NO: 10; a CDR-L2 comprising the amino acid sequence of SEQ ID NO: 11; a CDR-L3 comprising the amino acid sequence of SEQ ID NO: 12; V_{H2} comprises a CDR-H1 comprising the amino acid sequence of SEQ ID NO: 16; a CDR-H2 comprising the amino acid sequence of SEQ ID NO: 25; a CDR-H3 comprising the amino acid sequence of SEQ ID NO: 26; V_{L2} comprises a CDR-L1 comprising the amino acid sequence of SEQ ID NO: 39; a CDR-L2 comprising the amino acid sequence of SEQ ID NO: 31; a CDR-L3 comprising the amino acid sequence of SEQ ID NO: 40; and/or
(a) V_{H1} and V_{L1} corresponds to the amino acid sequences of SEQ ID NO: 41 and 43 respectively or corresponds to the amino acid sequences of SEQ ID NO: 42 and 44 respectively; and/or
(b) V_{H2} and V_{L2} corresponds to
the amino acid sequences of SEQ ID NO: 45 and 53 respectively;
the amino acid sequences of SEQ ID NO: 46 and 54 respectively;
the amino acid sequences of SEQ ID NO: 47 and 55 respectively;
the amino acid sequences of SEQ ID NO: 48 and 56 respectively;
the amino acid sequences of SEQ ID NO: 49 and 57 respectively;
the amino acid sequences of SEQ ID NO: 50 and 58 respectively;
the amino acid sequences of SEQ ID NO: 51 and 59 respectively; or
the amino acid sequences of SEQ ID NO: 52 and 60 respectively; optionally wherein
(a) V_{H1} comprises the amino acid sequence of SEQ ID NO: 41; V_{L1} comprises the amino acid sequence of SEQ ID NO: 43; V_{H2} comprises the amino acid sequence of SEQ ID NO: 45; V_{L2} comprises the amino acid sequence of SEQ ID NO: 53;
(b) V_{H1} comprises the amino acid sequence of SEQ ID NO: 41; V_{L1} comprises the amino acid sequence of SEQ ID NO: 43; V_{H2} comprises the amino acid sequence of SEQ ID NO: 46; V_{L2} comprises the amino acid sequence of SEQ ID NO: 54;
(c) V_{H1} comprises the amino acid sequence of SEQ ID NO: 41; V_{L1} comprises the amino acid sequence of SEQ ID NO: 43; V_{H2} comprises the amino acid sequence of SEQ ID NO: 47; V_{L2} comprises the amino acid sequence of SEQ ID NO: 55;
(d) V_{H1} comprises the amino acid sequence of SEQ ID NO: 41; V_{L1} comprises the amino acid sequence of SEQ ID NO: 43; V_{H2} comprises the amino acid sequence of SEQ ID NO: 48; V_{L2} comprises the amino acid sequence of SEQ ID NO: 56;
(e) V_{H1} comprises the amino acid sequence of SEQ ID NO: 41; V_{L1} comprises the amino acid sequence of SEQ ID NO: 43; V_{H2} comprises the amino acid sequence of SEQ ID NO: 49; V_{L2} comprises the amino acid sequence of SEQ ID NO: 57;
(f) V_{H1} comprises the amino acid sequence of SEQ ID NO: 41; V_{L1} comprises the amino acid sequence of SEQ ID NO: 43; V_{H2} comprises the amino acid sequence of SEQ ID NO: 50; V_{L2} comprises the amino acid sequence of SEQ ID NO: 58;
(g) V_{H1} comprises the amino acid sequence of SEQ ID NO: 41; V_{L1} comprises the amino acid sequence of SEQ ID NO: 43; V_{H2} comprises the amino acid sequence of SEQ ID NO: 51; V_{L2} comprises the amino acid sequence of SEQ ID NO: 59;
(h) V_{H1} comprises the amino acid sequence of SEQ ID NO: 41; V_{L1} comprises the amino acid sequence of SEQ ID NO: 43; V_{H2} comprises the amino acid sequence of SEQ ID NO: 52; V_{L2} comprises the amino acid sequence of SEQ ID NO: 60;
(i) V_{H1} comprises the amino acid sequence of SEQ ID NO: 42; V_{L1} comprises the amino acid sequence of SEQ ID NO: 44; V_{H2} comprises the amino acid sequence of SEQ ID NO: 45; V_{L2} comprises the amino acid sequence of SEQ ID NO: 53;
(j) V_{H1} comprises the amino acid sequence of SEQ ID NO: 42; V_{L1} comprises the amino acid sequence of SEQ ID NO: 44; V_{H2} comprises the amino acid sequence of SEQ ID NO: 46; V_{L2} comprises the amino acid sequence of SEQ ID NO: 54;
(k) V_{H1} comprises the amino acid sequence of SEQ ID NO: 42; V_{L1} comprises the amino acid sequence of SEQ ID NO: 44; V_{H2} comprises the amino acid sequence of SEQ ID NO: 47; V_{L2} comprises the amino acid sequence of SEQ ID NO: 55;
(l) V_{H1} comprises the amino acid sequence of SEQ ID NO: 42; V_{L1} comprises the amino acid sequence of SEQ ID NO: 44; V_{H2} comprises the amino acid sequence of SEQ ID NO: 48; V_{L2} comprises the amino acid sequence of SEQ ID NO: 56;
(m) V_{H1} comprises the amino acid sequence of SEQ ID NO: 42; V_{L1} comprises the amino acid sequence of SEQ ID NO: 44; V_{H2} comprises the amino acid sequence of SEQ ID NO: 49; V_{L2} comprises the amino acid sequence of SEQ ID NO: 57;
(n) V_{H1} comprises the amino acid sequence of SEQ ID NO: 42; V_{L1} comprises the amino acid sequence of SEQ ID NO: 44; V_{H2} comprises the amino acid sequence of SEQ ID NO: 50; V_{L2} comprises the amino acid sequence of SEQ ID NO: 58;
(o) V_{H1} comprises the amino acid sequence of SEQ ID NO: 42; V_{L1} comprises the amino acid sequence of SEQ ID NO: 44; V_{H2} comprises the amino acid sequence of SEQ ID NO: 51; V_{L2} comprises the amino acid sequence of SEQ ID NO: 59; or
V_{H1} comprises the amino acid sequence of SEQ ID NO: 42; V_{L1} comprises the amino acid sequence of SEQ ID NO: 44; V_{H2} comprises the amino acid sequence of SEQ ID NO: 52; V_{L2} comprises the amino acid sequence of SEQ ID NO: 60; and/or
polypeptide (I) comprises an amino acid sequence of SEQ ID NO: 64;
polypeptide (II) comprises an amino acid sequence of SEQ ID NO: 65; and
polypeptide (III) comprises an amino acid sequence of SEQ ID NO: 66.

115-118. (canceled)

119. The method of claim 84, wherein:
the leukemia is acute myeloid leukemia (AML), optionally wherein the AML is relapsed or refractory;
the leukemia is B cell acute lymphoblastic leukemia (B-ALL); and/or
the binding protein is administered to the subject intravenously, subcutaneously, intraperitoneally, or intramuscularly, optionally wherein the binding protein is administered to the subject intravenously.

120-141. (canceled)

142. The method of claim 85, wherein the subject is a pediatric subject.

143. The method of claim 85, wherein the binding protein comprises a first antigen binding domain with binding specificity to CD123, a second antigen binding domain with binding specificity to NKp46, and all or part of an immunoglobulin Fc region or variant thereof that binds to a human Fc-γ receptor, wherein the binding protein comprises:
a polypeptide (I) that comprises an amino acid sequence of SEQ ID NO: 64;
a polypeptide (II) that comprises an amino acid sequence of SEQ ID NO: 65; and
a polypeptide (III) that comprises an amino acid sequence of SEQ ID NO: 66.


SEQUENCE LISTING (116 sequences)

SEQ ID NO: 1 - protein, 10 aa, synthetic construct
GYSFTDYYMK

SEQ ID NO: 2 - protein, 10 aa, synthetic construct
DIIPSSGATF

SEQ ID NO: 3 - protein, 11 aa, synthetic construct
SHLLRASWFAY

SEQ ID NO: 4 - protein, 8 aa, synthetic construct
GFTFSHYN

SEQ ID NO: 5 - protein, 8 aa, synthetic construct
ITYDDHST

SEQ ID NO: 6 - protein, 10 aa, synthetic construct
ARLVNYAFAY

SEQ ID NO: 7 - protein, 17 aa, synthetic construct
ESSQSLLSSGNQKNYLT

SEQ ID NO: 8 - protein, 7 aa, synthetic construct
WASTRES

SEQ ID NO: 9 - protein, 9 aa, synthetic construct
QNDYSYPYT

SEQ ID NO: 10 - protein, 6 aa, synthetic construct
QTVGNN

SEQ ID NO: 11 - skipped

SEQ ID NO: 12 - protein, 8 aa, synthetic construct
QRMYNSPT

SEQ ID NO: 13 - protein, 5 aa, synthetic construct
DYVIN

SEQ ID NO: 14 - protein, 17 aa, synthetic construct
EIYPGSGTNYYNEKFKA

SEQ ID NO: 15 - protein, 11 aa, synthetic construct
RGRYGLYAMDY

SEQ ID NO: 16 - protein, 6 aa, synthetic construct
SDYAWN

SEQ ID NO: 17 - protein, 16 aa, synthetic construct
YITYSGSTSYNPSLES

SEQ ID NO: 18 - protein, 13 aa, synthetic construct
GGYYGSSWGVFAY

SEQ ID NO: 19 - protein, 5 aa, synthetic construct
EYTMH

SEQ ID NO: 20 - protein, 17 aa, synthetic construct
GISPNIGGTSYNQKFKG

SEQ ID NO: 21 - protein, 7 aa, synthetic construct
RGGSFDY

SEQ ID NO: 22 - protein, 5 aa, synthetic construct
SFTMH

SEQ ID NO: 23 - protein, 17 aa, synthetic construct
YINPSSGYTEYNQKFKD

SEQ ID NO: 24 - protein, 8 aa, synthetic construct
GSSRGFDY

SEQ ID NO: 25 - protein, 16 aa, synthetic construct
YITYSGSTNYNPSLKS

SEQ ID NO: 26 - protein, 11 aa, synthetic construct
CWDYALYAMDC

SEQ ID NO: 27 - protein, 11 aa, synthetic construct
RASQDISNYLN

SEQ ID NO: 28 - protein, 7 aa, synthetic construct
YTSRLHS

SEQ ID NO: 29 - protein, 9 aa, synthetic construct
QQGNTRPWT

SEQ ID NO: 30 - protein, 11 aa, synthetic construct
RVSENIYSYLA

SEQ ID NO: 31 - protein, 7 aa, synthetic construct
NAKTLAE

SEQ ID NO: 32 - protein, 9 aa, synthetic construct
QHHYGTPWT

SEQ ID NO: 33 - protein, 11 aa, synthetic construct
RASQSISDYLH

SEQ ID NO: 34 - protein, 7 aa, synthetic construct
YASQSIS

SEQ ID NO: 35 - protein, 9 aa, synthetic construct
QNGHSFPLT

SEQ ID NO: 36 - protein, 11 aa, synthetic construct
RASENIYSNLA

SEQ ID NO: 37 - protein, 7 aa, synthetic construct
AATNLAD

SEQ ID NO: 38 - protein, 9 aa, synthetic construct
QHFWGTPRT

SEQ ID NO: 39 - protein, 11 aa, synthetic construct
RTSENIYSYLA

SEQ ID NO: 40 - protein, 9 aa, synthetic construct
QHHYDTPLT

SEQ ID NO: 41 - protein, 120 aa, synthetic construct [sequence omitted]

SEQ ID NO: 42 - protein, 117 aa, synthetic construct
EVQLVESGGGLVQPGRSLKLSCAASGFTFSHYNMAWVRQAPKKGLEWVATITYDDHSTYYRDSVKGRFTISRDTAKSTLYLQMDSLRSEDTATYYCARLVNYAFAYWGQGTLVTVSS

SEQ ID NO: 43 - protein, 113 aa, synthetic construct [sequence omitted]

SEQ ID NO: 44 - protein, 106 aa, synthetic construct
NIVMTQSPKSMSISVGDRVTMNCKASQTVGNNIAWYQQKPGLSPQLLIDYASNRYTGVPNRFTGGGYGTDFILTINSVQAEDAAFYYCQRMYNSPTFGGGTKLELK

SEQ ID NO: 45 - protein, 120 aa, synthetic construct
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 46 - protein, 120 aa, synthetic construct
QVQLQQSGPELVKPGASVKMSCKASGYTFTDYVINWGKQRSGQGLEWIGEIYPGSGTNYYNEKFKAKATLTADKSSNIAYMQLSSLTSEDSAVYFCARRGRYGLYAMDYWGQGTSVTVSS

SEQ ID NO: 47 - protein, 122 aa, synthetic construct
EVQLQESGPGLVKPSQSLSLTCTVTGYSITSDYAWNWIRQFPGNKLEWMGYITYSGSTSYNPSLESRISITRDTSTNQFFLQLNSVTTEDTATYYCARGGYYGSSWGVFAYWGQGTLVTVSA

SEQ ID NO: 48 - protein, 116 aa, synthetic construct
EVQLQQSGPELVKPGASVKISCKTSGYTFTEYTMHWVKQSHGKSLEWIGGISPNIGGTSYNQKFKGKATLTVDKSSSTAYMELRSLTSEDSAVYYCARRGGSFDYWGQGTTLTVSS

SEQ ID NO: 49 - protein, 117 aa, synthetic construct
QVQLQQSAVELARPGASVKMSCKASGYTFTSFTMHWVKQRPGQGLEWIGYINPSSGYTEYNQKFKDKTTLTADKSSSTAYMQLDSLTSDDSAVYYCVRGSSRGFDYWGQGTLVTVSA

SEQ ID NO: 50 - protein, 122 aa, synthetic construct
QVQLQESGPGLVKPSQTLSLTCTVSGYSISSDYAWNWIRQPPGKGLEWIGYITYSGSTSYNPSLESRVTISRDTSKNQFSLKLSSVTAADTAVYYCARGGYYGSSWGVFAYWGQGTLVTVSS

SEQ ID NO: 51 - protein, 117 aa, synthetic construct
QVQLVQSGAEVKKPGASVKVSCKASGYTFTSFTMHWVRQAPGQGLEWIGYINPSSGYTEYNQKFKDRVTITADKSTSTAYMELSSLRSEDTAVYYCVRGSSRGFDYWGQGTLVTVSS

SEQ ID NO: 52 - protein, 120 aa, synthetic construct
QVQLQESGPGLVKPSQTLSLTCTVSGYSISSDYAWNWIRQPPGKGLEWIGYITYSGSTNYNPSLKSRVTISRDTSKNQFSLKLSSVTAADTAVYYCARCWDYALYAMDCWGQGTTVTVSS

SEQ ID NO: 53 - protein, 107 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 54 - protein, 107 aa, synthetic construct
DIQMTQTTSSLSASLGDRVTISCRASQDISNYLNWYQQKPDGTVKLLIYYTSRLHSGVPSRFSGSGSGTDYSLTINNLEQEDIATYFCQQGNTRPWTFGGGTKLEIK

SEQ ID NO: 55 - protein, 107 aa, synthetic construct
DIQMTQSPASLSASVGETVTITCRVSENIYSYLAWYQQKQGKSPQLLVYNAKTLAEGVPSRFSGSGSGTQFSLKINSLQPEDFGSYYCQHHYGTPWTFGGGTKLEIK

SEQ ID NO: 56 - protein, 107 aa, synthetic construct
DIVMTQSPATLSVTPGDRVSLSCRASQSISDYLHWYQQKSHESPRLLIKYASQSISGIPSRFSGSGSGSDFTLSINSVEPEDVGVYYCQNGHSFPLTFGAGTKLELK

SEQ ID NO: 57 - protein, 107 aa, synthetic construct
DIQMIQSPASLSVSVGETVTITCRASENIYSNLAWFQQKQGKSPQLLVYAATNLADGVPSRFSGSGSGTQYSLKINSLQSEDFGIYYCQHFWGTPRTFGGGTKLEIK

SEQ ID NO: 58 - protein, 107 aa, synthetic construct
DIQMTQSPSSLSASVGDRVTITCRVSENIYSYLAWYQQKPGKAPKLLVYNAKTLAEGVPSRFSGSGSGTDFTLTISSLQPEDFATYYCQHHYGTPWTFGGGTKVEIK

SEQ ID NO: 59 - protein, 107 aa, synthetic construct
DIQMTQSPSSLSASVGDRVTITCRASENIYSNLAWFQQKPGKAPKLLVYAATNLADGVPSRFSGSGSGTDYTLTISSLQPEDFATYYCQHFWGTPRTFGGGTKVEIK

SEQ ID NO: 60 - protein, 107 aa, synthetic construct
DIQMTQSPSSLSASVGDRVTITCRTSENIYSYLAWCQQKPGKAPKLLIYNAKTLAEGVPSRFSGSGSGTDFTLTISSLQPEDFATYYCQHHYDTPLTFGQGTKLEIK

SEQ ID NO: 61 - protein, 467 aa, synthetic construct
MSVPTQVLGLLLLWLTDARCDIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 700 aa, synthetic construct
MEWSWVFLFFLSVTTGVHSEVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 63 - protein, 234 aa, synthetic construct
MSVPTQVLGLLLLWLTDARCDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 447 aa, synthetic construct
DIVMTQSPDSLAVSLGERATINCESSQSLLSSGNQKNYLTWYQQKPGQPPKPLIYWASTRESGVPDRFSGSGSGTDFTLTISSLQAEDVAVYYCQNDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 681 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 66 - protein, 214 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 107 aa, synthetic construct [sequence omitted]

SEQ ID NO: 68 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 69 - protein, 217 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 216 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 110 aa, synthetic construct
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 72 - protein, 107 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 106 aa, synthetic construct
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 10 aa, synthetic construct
DKTHTCPPCP

SEQ ID NO: 75 - protein, 15 aa, synthetic construct
EPKSCDKTHTCPPCP

SEQ ID NO: 76 - protein, 4 aa, synthetic construct
STGS

SEQ ID NO: 77 - protein, 10 aa, synthetic construct
EPKSCDKTHS

SEQ ID NO: 78 - protein, 9 aa, synthetic construct
EPKSCDKTH

SEQ ID NO: 79 - protein, 10 aa, synthetic construct
EPKSCDKTHT

SEQ ID NO: 80 - protein, 255 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQN

SEQ ID NO: 81 - protein, 262 aa, Macaca fascicularis
MSSTLRALLCLGLCLSQRISAPKQTLPKPIIRAESTYMVPKEKQATLCCQGSYGAVEYQLHFEGSLFAVERPKPPERINGVKFHIPDMNSRKAGRYSCIYRVGELWSERSDLLDLVVTEMYDTPTLSVHPGPEVTSGEKVTFYCRLDTATSMFLLLKEGRSRDVQRSYGKVQAEFPMGPVTTAHRGSYRCFGSYNNYAWSFPSEPVKLLVTGDIENTSLAPTDPTFPDSWDTCLLTRETGLQKDLALWDHTAQNDYKDDDDK

SEQ ID NO: 82 - protein, 287 aa, Homo sapiens
TKEDPNPPITNLRMKAKAQQLTWDLNRNVTDIECVKDADYSMPAVNNSYCQFGAISLCEVTNYTVRVANPPFSTWILFPENSGKPWAGAENLTCWIHDVDFLSCSWAVGPGAPADVQYDLYLNVANRRQQYECLHYKTDAQGTRIGCRFDDISRLSSGSQSSHILVRGRSAAFGIPCTDKFVVFSQIEILTPPNMTAKCNKTHSFMHWKMRSHFNRKFRYELQIQKRMQPVITEQVRDRTSFQLLNPGTYTVQIRARERVYEFLSAWSTPQRFECDQEEGANTRAWR

SEQ ID NO: 83 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 84 - protein, 254 aa, synthetic construct
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 85 - protein, 441 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 678 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 87 - protein, 214 aa, synthetic construct; identical to SEQ ID NO: 66

SEQ ID NO: 88 - protein, 447 aa, synthetic construct; identical to SEQ ID NO: 64

SEQ ID NO: 89 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARGTTIFNYFEYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 90 - protein, 220 aa, synthetic construct
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYEIPPTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 447 aa, synthetic construct; identical to SEQ ID NO: 64

SEQ ID NO: 92 - protein, 680 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 215 aa, synthetic construct
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 94 - protein, 447 aa, synthetic construct; identical to SEQ ID NO: 64

SEQ ID NO: 95 - protein, 679 aa, synthetic construct
EVQLVQSGAEVKKPGESLKISCKGSGYSFTDYYMKWARQMPGKGLEWMGDIIPSSGATFYNQKFKGQVTISADKSISTTYLQWSSLKASDTAMYYCARSHLLRASWFAYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSEVQLVQSGAEVKKSGESLKISCKGSGYSFTSYWIGWVRQMPGKGLEWMGIFYPGDSSTRYSPSFQGQVTISADKSVNTAYLQWSSLKASDTAMYYCARRRNWGNAFDIWGQGTMVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 216 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSTWTFGQGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 97 - protein, 214 aa, synthetic construct
EIVLTQSPATLSLSPGERATLSCRASQSVRSYLAWYQQKPGQAPRLLFSDASNRATGIPARFSGSGSGTDFTLTISSLEPEDFAVYYCQQYRYSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 447 aa, synthetic construct
EVQLVESGGGLVQPGRSLRLSCAASGFTFDNYAMHWVRQAPGKGLEWVSGISRSSGDIDYADSVKGRFTISRDNAKNSLYLQMNSLRAEDTALYYCARGGVGSFDTWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 6 aa, synthetic construct
QDISNY

SEQ ID NO: 100 - skipped

SEQ ID NO: 101 - protein, 10 aa, synthetic construct
QQGNTRPWTF

SEQ ID NO: 102 - protein, 8 aa, synthetic construct
GYTFSDYV

SEQ ID NO: 103 - protein, 8 aa, synthetic construct
IYPGSGTN

SEQ ID NO: 104 - protein, 13 aa, synthetic construct
ARRGRYGLYAMDY

SEQ ID NO: 105 - protein, 7 aa, synthetic construct
SQDISNY

SEQ ID NO: 106 - skipped

SEQ ID NO: 107 - protein, 6 aa, synthetic construct
GNTRPW

SEQ ID NO: 108 - protein, 7 aa, synthetic construct
GYTFSDY

SEQ ID NO: 109 - protein, 4 aa, synthetic construct
PGSG

SEQ ID NO: 110 - protein, 9 aa, synthetic construct
GRYGLYAMD

SEQ ID NO: 111 - protein, 8 aa, synthetic construct
ASQDISNY

SEQ ID NO: 112 - protein, 12 aa, synthetic construct
YTSRLHSGVPSR

SEQ ID NO: 113 - protein, 10 aa, synthetic construct
ASGYTFSDYV

SEQ ID NO: 114 - protein, 17 aa, synthetic construct
IYPGSGTNYYNEKFKAK

SEQ ID NO: 115 - protein, 10 aa, synthetic construct
RGRYGLYAMD

SEQ ID NO: 116 - protein, 9 aa, synthetic construct
QQGNTRPWT